SEQUENCE LISTING (14 sequences)

SEQ ID NO: 1 - protein, 5 aa, synthetic construct
GGGGS

SEQ ID NO: 2 - protein, 10 aa, synthetic construct
GGGGSGGGGS

SEQ ID NO: 3 - protein, 15 aa, synthetic construct
GGGGSGGGGSGGGGS

SEQ ID NO: 4 - protein, 20 aa, synthetic construct
GGGGSGGGGSGGGGSGGGGS

SEQ ID NO: 5 - protein, 8 aa, synthetic construct
GGGGGGGG

SEQ ID NO: 6 - protein, 6 aa, synthetic construct
GGGGGG

SEQ ID NO: 7 - protein, 15 aa, synthetic construct
EAAAKEAAAKEAAAK

SEQ ID NO: 8 - protein, 4 aa, synthetic construct
EAAK

SEQ ID NO: 9 - protein, 8 aa, synthetic construct
EAAKEAAK

SEQ ID NO: 10 - protein, 12 aa, synthetic construct
EAAKEAAKEAAK

SEQ ID NO: 11 - protein, 46 aa, synthetic construct
AEAAAKEAAAKEAAAKEAAAKALEAEAAAKEAAAKEAAAKEAAAKA

SEQ ID NO: 12 - protein, 12 aa, synthetic construct
AEAAAKEAAAKA

SEQ ID NO: 13 - protein, 6 aa, synthetic construct
GGGGSG

SEQ ID NO: 14 - protein, 6 aa, synthetic construct
LALAGA

(19) United States
(12) Patent Application Publication
(10) Pub. No.: US 2023/0272072 A1
(43) Pub. Date: Aug. 31, 2023

(54) DUAL TARGETED IMMUNE REGULATING COMPOSITIONS

(71) Applicant: Seismic Therapeutic, Inc., Watertown, MA (US)

(72) Inventors: Joanne L. Viney, Belmont, MA (US); Nathan Higginson-Scott, Boston, MA (US)

(21) Appl. No.: 18/048,747

(22) Filed: Oct. 21, 2022

Related U.S. Application Data
(60) Provisional application No. 63/262,830, filed on Oct. 21, 2021.

(51) Int. Cl. C07K 16/28; A61P 37/02; A61P 35/00

(52) U.S. Cl. CPC C07K 16/2803; A61P 37/02; A61P 35/00; C07K 2317/52; C07K 2317/71

(57) ABSTRACT

Embodiments provided herein, provide for polypeptides, pharmaceutical compositions, and methods that can be used to target at least two types of cells to modulate the activity of the same to treat disorders, such as autoimmune disorders or cancers.

### CROSS-REFERENCE TO RELATED APPLICATIONS

This application claims priority to U.S. Provisional Application No. 63/262,830, filed Oct. 21, 2021, which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Oct. 11, 2022, is named “258618_000101_Seq.XML” and is 13,217 bytes in size.

### FIELD

The embodiments provided herein relate to compositions that target different cells to regulate an immune response.

### BACKGROUND

Cell-mediated immunity plays a critical role in the body's immune response. Unfortunately, uncontrolled cell-mediated immunity may lead to disease or auto-immune conditions. Most treatments available today regulate the body's immune response by targeting one factor. However, these treatments are not always effective, and, therefore, there is still a need for treatments that regulate cell-mediated immunity. In contrast, in treating cancers, there is a need to activate the body's immune response to target the cancer cells. The molecules immuno-oncology products approved today generally only target one type of cell through the binding of a single receptor, which can lead to an incomplete activation of an immune response to treat such cancers. The embodiments provided for herein fulfill these needs as well as others.

### SUMMARY

In some embodiments, polypeptides comprising an inhibitory receptor effector domain, a Fc region, and a FcγRII binding effector domain are provided. In some embodiments, polypeptides comprising an inhibitory receptor effector domain and a FcγRII binding effector domain are provided. In some embodiments, polypeptides comprising a plurality of inhibitory receptor effector domains each linked to a Fc region are provided. In some embodiments, the polypeptide comprises 1, 2, 3, or 4 inhibitory receptor effector domains.

In some embodiments, the inhibitory receptor effector domains bind to the same inhibitory receptor. In some embodiments, the inhibitory receptor effector domains bind to different inhibitory receptor. In some embodiments, the 2 inhibitory receptor effector domains bind to the same inhibitory receptor. In some embodiments, the 2 inhibitory receptor effector domains bind to the same inhibitory receptor, wherein the inhibitory receptor effector domain binds to an epitope on the inhibitory receptor, and the second inhibitory receptor effector domain binds to a different epitope on the same inhibitory receptor. In some embodiments, the 2 inhibitory receptor effector domains bind to different inhibitory receptors. In some embodiments, the inhibitory receptor effector domain is an antibody. In some embodiments, the inhibitory receptor effector domain is an antibody in the format of a scFv, Fab, Fab′, and F(ab′)2 antibody. In some embodiments, the inhibitory receptor effector domain binds to PD-1, LAG-3, or CTLA4. In some embodiments, the inhibitory receptor effector domain binds to a receptor encoded by LAG3, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PDCD1, PECAM1/CD31, PILRA, PVR/CD155, SIGLEC11, SIGLECS, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, ILT3, ILT4, ILT5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CDS, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.

In some embodiments, the inhibitory receptor effector domain is an agonist of the inhibitory receptor to which it binds. In some embodiments, the inhibitory receptor effector domain is an antagonist of the inhibitory receptor to which it binds.

In some embodiments, the Fc region is selective for FcγRIIb. In some embodiments, the Fc region comprises FcγRIIb selective mutations, such as those provided for herein. In some embodiments, the Fc region is selective for FcγRIIb over FcγRIIa. In some embodiments, the Fc region comprises mutations, such as those provided for herein, wherein said mutations confer selectivity for FcγRIIb over FcγRIIa.

In some embodiments, the FcγRII binding effector domains binds to FcγRIIb or FcγRIIa. In some embodiments, the FcγRII binding effector domains is an antibody. In some embodiments, the antibody is an scFv, Fab, Fab′, and F(ab′)2.

In some embodiments, pharmaceutical compositions are provided that comprise a polypeptide as provided for herein.

In some embodiments, methods of treating an autoimmune disorder in a subject are provided.

In some embodiments, methods of treating cancer in a subject are provided.

In some embodiments, methods of modulating two types of cells with a polypeptide as provided for herein are provided. In some embodiments, one cell is a T-cell, NK Cell, Dendritic cell, and the like and the second cell is a B-Cell, an antigen presenting cell (APC), or a myeloid cell.

In some embodiments, methods of modulating the activity of two types of cells in a subject are provided. In some embodiments, one cell is a T-cell, NK Cell, Dendritic cell, and the like and the second cell is a B-Cell, an antigen presenting cell (APC), or a myeloid cell.

In some embodiments, methods of inhibiting an activated immune cell (e.g. T-cell, NK Cell, Dendritic cell, and the like) and the activity of a B-Cell, an antigen presenting cell (APC), or a myeloid cell are provided.

In some embodiments, methods of activating or enhancing an activated immune cell (e.g. T-cell, NK Cell, Dendritic cell, and the like) and the activity of B-Cell, an antigen presenting cell (APC), or a myeloid cell are provided.

### BRIEF DESCRIPTION OF THE DRAWINGS

FIG. 1 illustrates non-limiting embodiments as provided for herein.

FIG. 1 depicts non-limiting embodiments of the therapeutic compounds provided herein.

FIGS. 2A and 2B each depicts a non-limiting illustration of how a therapeutic compound provided herein could function.

FIG. 3 depicts a non-limiting illustration of the therapeutic compounds provided herein.

FIG. 3A depicts a non-limiting illustration of the therapeutic compounds provided herein.

FIG. 4 depicts a non-limiting illustration of the therapeutic compounds provided herein.

FIG. 5 depicts a non-limiting illustration of the therapeutic compounds provided herein.

FIG. 6 depicts a non-limiting illustration of the therapeutic compounds provided herein.

FIG. 7 depicts a non-limiting illustration of the therapeutic compounds provided herein.

FIG. 8 depicts a non-limiting illustration of the therapeutic compounds provided herein.

FIG. 9 depicts a non-limiting illustration of the therapeutic compounds provided herein.

FIG. 10 depicts a non-limiting illustration of the therapeutic compounds provided herein.

FIG. 11 depicts a non-limiting illustration of the therapeutic compounds provided herein.

FIG. 12 depicts a non-limiting illustration of the therapeutic compounds provided herein.

FIG. 13 depicts a non-limiting illustration of the therapeutic compounds provided herein.

FIG. 14 depicts a non-limiting illustration of the therapeutic compounds provided herein.

FIG. 15 illustrates binding affinities of various test articles.

FIG. 16 illustrates PD-1 agonism of various test articles.

FIG. 17 illustrates PD-1 agonism of various test articles in presence or absence of FcγRIIb.

### DETAILED DESCRIPTION

As used herein and unless otherwise indicated, the term “about” means that the numerical value is approximate and small variations would not significantly affect the practice of the disclosed embodiment. Where a numerical limitation is used, unless indicated otherwise by the context, “about” means the numerical value can vary by ±10% and remain within the scope of the disclosed embodiments.

As used herein and in the appended claims, the singular forms “a”, “an” and “the” include plural reference unless the context clearly dictates otherwise.

As used herein, the term “animal” includes, but is not limited to, humans and non-human vertebrates such as wild, domestic, and farm animals. Accordingly, as used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound or composition to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” which can be used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. An result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

As used herein, the term “position,” is meant to refer to a location in the sequence of a polypeptide. Positions may be numbered sequentially, or according to an established format, for example the EU numbering system based on Kabat's amino acid positions. For example, position 298 is a position in the human antibody IgG1.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10⁻⁴ m, at least about 10⁻⁵ m, at least about 10⁻⁶ m, at least about 10⁻⁷ m, at least about 10⁻⁸ m, at least about 10⁻⁹ m, alternatively at least about 10⁻¹⁰ M, at least about 10⁻¹¹ m, at least about 10⁻¹² m, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the compounds and compositions provided for herein can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival, as applicable for a specific disease, as compared to expected survival if not receiving treatment. Thus, “treatment of an autoimmune condition” or “treating autoimmunity” means an activity that alleviates or ameliorates any of the primary phenomena or secondary symptoms associated with the autoimmune condition other condition described herein when the terms “treat,” “treated,” or “treating” are used in conjunction with such condition.

Provided herein are compounds, such as polypeptides or fusion proteins, e.g., that can be used as therapeutics that include two or more effector domains that bind to at least two different immune cell types. In some embodiments, the compound comprises 2, 3, or 4 effector domains, such as an inhibitory receptor effector domain. In some embodiments, the compounds binds to at least 2 different cell surface receptors molecules, with at least one being on two different cell types. In some embodiments, the compound can comprise 3 effector domains, wherein at least two of the effector domains, which can be inhibitory receptor effector domains, bind to different cell surface receptors, but the at least two of the effector domains bind to the different cell surface receptors on the same cell or cell type. For example, a polypeptide can comprise an inhibitory receptor effector domain that binds to PD-1 and a second inhibitory receptor effector domain that binds to LAG-3. The interaction of these domains with PD-1 and LAG-3 can be, for example on the same cell or it can be on the same cell type, but wherein the PD-1 and LAG-3 are on different cells. In some embodiments, the effector domains can modulate the activity of the cell that they bind to by modulating the activity of the cell surface receptor to which they bind to. In some embodiments, each effector domain, independently, agonizes the activity of molecule to which it binds to. In some embodiments, each effector domain, independently, antagonizes the activity of molecule to which it binds to.

Without being bound to any particular theory, the effector domains by binding to two different cells at the same time, nearly the same time, or in the same local environment, the compounds provided herein can modulate the cell-mediated immunity being regulated by those cells. In some embodiments, the immune response is suppressed. In some embodiments, the immune response is activated. When the immune response is suppressed, the polypeptide can be used to, for example, treat an auto-immune disease or condition, such as those provided for herein. When the immune response is activated, the polypeptide can be used to, for example, treat cancer or other proliferative disorder, such as those provided for herein.

Also provided are methods of using and making the compounds.

In some embodiments, a polypeptide is provided that comprises: a) an inhibitory receptor effector domain; b) a Fc domain; and c) a FcγRII binding effector domain. In some embodiments, a polypeptide is provided that comprises: a) an inhibitory receptor effector domain and b) a Fc domain. In some embodiments, a polypeptide is provided that comprises: a) an inhibitory receptor effector domain and b) a FcγRII binding effector domain. In some embodiments, a polypeptide is provided that comprises: a) an inhibitory receptor effector domain; b) a Fc domain; and c) a FcγRII binding effector domain. In some embodiments, a polypeptide is provided that comprises an inhibitory receptor effector domain and a FcγRII binding effector domain, i.e., without an Fc domain. In some embodiments, a polypeptide is provided that comprises a plurality of inhibitory receptor effector domains and a Fc domain linked to each inhibitory receptor effector domain. The Fc domains linked to each inhibitory receptor effector domain can be the same or different. In some embodiments, a polypeptide is provided that comprises 1, 2, 3, or 4 inhibitory receptor effector domains, each linked to a Fc domain. The Fc domains linked to each inhibitory receptor effector domain can be the same or different. In some embodiments, the inhibitory receptor domains are linked to the Fc domain to the N-terminus and/or the C-terminus of the Fc domain. In some embodiments, each Fc domain has 1 or 2 inhibitory receptor domains linked to the Fc domain. In some embodiments, the Fc domain has an inhibitory effector domain linked to the N-terminus and the C-terminus of the Fc domain. In some embodiments, the inhibitory effector domains binds to the same inhibitory receptor. In some embodiments, the inhibitory effector domain binds to different inhibitory receptors.

In some embodiments, the polypeptide comprises from the N-terminus to the C-terminus: a) an inhibitory receptor effector domain; b) a Fc domain; and c) a FcγRII binding effector domain. In some embodiments, the polypeptide comprises from the N-terminus to the C-terminus a) a FcγRII binding effector domain b) a Fc domain; and c) an inhibitory receptor effector domain.

In some embodiments, the polypeptide comprises from the N-terminus to the C-terminus:

an inhibitory receptor effector domain and a FcγRII binding effector domain. In some embodiments, the polypeptide comprises from the N-terminus to the C-terminus: a FcγRII binding effector domain and an inhibitory receptor effector domain.

In some embodiments, the polypeptide comprises from the N-terminus to the C-terminus: a) an inhibitory receptor effector domain and a Fc domain. In some embodiments, the polypeptide comprises from the N-terminus to the C-terminus a) Fc domain and an inhibitory receptor effector domain.

In each of the embodiments, provided for herein, the domains can be linked to one another with a peptide linker, such as the non-limiting examples provided for herein, or without an intervening peptide linker.

In some embodiments, the polypeptide comprises a plurality of inhibitory receptor effector domains that can bind to either the same inhibitory receptors or to two different inhibitory receptors. As provided for herein, in some embodiments, the polypeptide comprises two inhibitory receptor effector domains that bind to the same or different inhibitory receptors.

As used herein, the term “inhibitory receptor effector domain” refers to a polypeptide, such as an antibody, that binds to an inhibitory receptor present on an immune cell, such as, but not limited to, T-cells. In some embodiments, the T-cell is an activated T-cell. In some embodiments, the T-cell is not activated. In some embodiments, the polypeptide comprises one or more inhibitory receptor effector domains. In some embodiments, the polypeptide comprises 2, 3, or 4 inhibitory receptor effector domains. In some embodiments, the inhibitory receptor effector domains bind to the same inhibitory receptors. In some embodiments, the different inhibitory receptor effector domains bind to different inhibitory receptors. For example, if the polypeptide comprises two inhibitory receptor effector domains that bind to different inhibitory receptors, the first inhibitory receptor effector domain can bind to a first inhibitory receptor and the second inhibitory receptor effector domain can bind to a second inhibitory receptor that is different from the first. In some embodiments, the inhibitory receptor effector domain is an antibody. In some embodiments, the antibody is a Fab format antibody. In some embodiments, the antibody is a scFv antibody. In some embodiments, the antibody is an antibody as provided for herein. In some embodiments, the polypeptide comprises an inhibitory receptor effector domain that is an antibody in a Fab format and an inhibitory receptor effector domain that is an scFv antibody.

In some embodiments, the inhibitory receptor effector domain binds and modulates the activity of inhibitory receptors encoded by the genes: LAG3, PDCD1, LAG3, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLECS, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, ILT3, ILT4, ILT5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CDS, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.

In some embodiments, the inhibitory receptor effector domain binds and modulates the activity of the inhibitory receptor encoded by BTLA/CD272. In some embodiments, the inhibitory receptor effector domain binds and modulates the activity of the inhibitory receptor encoded by CD200R1. In some embodiments, the inhibitory receptor effector domain binds and modulates the activity of the inhibitory receptor encoded by CD22/Siglec2. In some embodiments, the inhibitory receptor effector domain binds and modulates the activity of the inhibitory receptor encoded by CD300A. In some embodiments, the inhibitory receptor effector domain binds and modulates the activity of the inhibitory receptor encoded by CD300LF/CD300F. In some embodiments, the inhibitory receptor effector domain binds and modulates the activity of the inhibitory receptor encoded by CD33/Siglec3. In some embodiments, the inhibitory receptor effector domain binds and modulates the activity of the inhibitory receptor encoded by CD5. In some embodiments, the inhibitory receptor effector domain binds and modulates the activity of the inhibitory receptor encoded by CD72. In some embodiments, the inhibitory receptor effector domain binds and modulates the activity of the inhibitory receptor encoded by CEACAM 1. In some embodiments, the inhibitory receptor effector domain binds and modulates the activity of the inhibitory receptor encoded by CLEC12A. In some embodiments, the inhibitory receptor effector domain binds and modulates the activity of the inhibitory receptor encoded by CLEC4A. In some embodiments, the inhibitory receptor effector domain binds and modulates the activity of the inhibitory receptor encoded by CTLA4/CD152. In some embodiments, the inhibitory receptor effector domain binds and modulates the activity of the inhibitory receptor encoded by FCGR2B/CD32B. In some embodiments, the inhibitory receptor effector domain binds and modulates the activity of the inhibitory receptor encoded by KIRs. In some embodiments, the inhibitory receptor effector domain binds and modulates the activity of the inhibitory receptor encoded by KLRB1/CD161. In some embodiments, the inhibitory receptor effector domain binds and modulates the activity of the inhibitory receptor encoded by KLRC1. In some embodiments, the inhibitory receptor effector domain binds and modulates the activity of the inhibitory receptor encoded by KLRG1. In some embodiments, the inhibitory receptor effector domain binds and modulates the activity of the inhibitory receptor encoded by LAIR1. In some embodiments, the inhibitory receptor effector domain binds and modulates the activity of the inhibitory receptor encoded by LILRB1. In some embodiments, the inhibitory receptor effector domain binds and modulates the activity of the inhibitory receptor encoded by LILRB2. In some embodiments, the inhibitory receptor effector domain binds and modulates the activity of the inhibitory receptor encoded by LILRB4. In some embodiments, the inhibitory receptor effector domain binds and modulates the activity of the inhibitory receptor encoded by LILRB5. In some embodiments, the inhibitory receptor effector domain binds and modulates the activity of the inhibitory receptor encoded by NCR2/NKp44. In some embodiments, the inhibitory receptor effector domain binds and modulates the activity of the inhibitory receptor encoded by PDCD1. In some embodiments, the inhibitory receptor effector domain binds and modulates the activity of the inhibitory receptor encoded by PECAM1/CD31. In some embodiments, the inhibitory receptor effector domain binds and modulates the activity of the inhibitory receptor encoded by PILRA. In some embodiments, the inhibitory receptor effector domain binds and modulates the activity of the inhibitory receptor encoded by PVR/CD155. In some embodiments, the inhibitory receptor effector domain binds and modulates the activity of the inhibitory receptor encoded by SIGLEC11. In some embodiments, the inhibitory receptor effector domain binds and modulates the activity of the inhibitory receptor encoded by SIGLEC5. In some embodiments, the inhibitory receptor effector domain binds and modulates the activity of the inhibitory receptor encoded by SIGLEC7. In some embodiments, the inhibitory receptor effector domain binds and modulates the activity of the inhibitory receptor encoded by SIGLEC8. In some embodiments, the inhibitory receptor effector domain binds and modulates the activity of the inhibitory receptor encoded by SIGLEC9. In some embodiments, the inhibitory receptor effector domain binds and modulates the activity of the inhibitory receptor encoded by SIRPA. In some embodiments, the inhibitory receptor effector domain binds and modulates the activity of the inhibitory receptor encoded by TIGIT. In some embodiments, the inhibitory receptor effector domain binds and modulates the activity of the inhibitory receptor encoded by VSTM1/SIRL1. In some embodiments, the inhibitory receptor effector domain binds and modulates the activity of the inhibitory receptor encoded by MAFA. In some embodiments, the inhibitory receptor effector domain binds and modulates the activity of the inhibitory receptor encoded by NKG2A. In some embodiments, the inhibitory receptor effector domain binds and modulates the activity of the inhibitory receptor encoded by CMRF35H. In some embodiments, the inhibitory receptor effector domain binds and modulates the activity of the inhibitory receptor encoded by CD66a. In some embodiments, the inhibitory receptor effector domain binds and modulates the activity of the inhibitory receptor encoded by CD66d. In some embodiments, the inhibitory receptor effector domain binds and modulates the activity of the inhibitory receptor encoded by CD33. In some embodiments, the inhibitory receptor effector domain binds and modulates the activity of the inhibitory receptor encoded by SIGLEC6. In some embodiments, the inhibitory receptor effector domain binds and modulates the activity of the inhibitory receptor encoded by ILT2. In some embodiments, the inhibitory receptor effector domain binds and modulates the activity of the inhibitory receptor encoded by ILT3. In some embodiments, the inhibitory receptor effector domain binds and modulates the activity of the inhibitory receptor encoded by ILT4. In some embodiments, the inhibitory receptor effector domain binds and modulates the activity of the inhibitory receptor encoded by ILT5. In some embodiments, the inhibitory receptor effector domain binds and modulates the activity of the inhibitory receptor encoded by LIRE. In some embodiments, the inhibitory receptor effector domain binds and modulates the activity of the inhibitory receptor encoded by KIR2DL. In some embodiments, the inhibitory receptor effector domain binds and modulates the activity of the inhibitory receptor encoded by KIR3DL. In some embodiments, the inhibitory receptor effector domain binds and modulates the activity of the inhibitory receptor encoded by SIRPa. In some embodiments, the inhibitory receptor effector domain binds and modulates the activity of the inhibitory receptor encoded by KIR2DL2/3. In some embodiments, the inhibitory receptor effector domain binds and modulates the activity of the inhibitory receptor encoded by KIR2DL5. In some embodiments, the inhibitory receptor effector domain binds and modulates the activity of the inhibitory receptor encoded by KIRDL1. In some embodiments, the inhibitory receptor effector domain binds and modulates the activity of the inhibitory receptor encoded by KIRDL2. In some embodiments, the inhibitory receptor effector domain binds and modulates the activity of the inhibitory receptor encoded by KIRDL3. In some embodiments, the inhibitory receptor effector domain binds and modulates the activity of the inhibitory receptor encoded by TIM3. In some embodiments, the inhibitory receptor effector domain binds and modulates the activity of the inhibitory receptor encoded by Tactile. In some embodiments, the inhibitory receptor effector domain binds and modulates the activity of the inhibitory receptor encoded by IRp60. In some embodiments, the inhibitory receptor effector domain binds and modulates the activity of the inhibitory receptor encoded by NKRP1. In some embodiments, the inhibitory receptor effector domain binds and modulates the activity of the inhibitory receptor encoded by IAP. In some embodiments, the inhibitory receptor effector domain binds and modulates the activity of the inhibitory receptor encoded by PIR-B. In some embodiments, the inhibitory receptor effector domain binds and modulates the activity of the inhibitory receptor encoded by 2B4. In some embodiments, the inhibitory receptor effector domain binds and modulates the activity of the inhibitory receptor encoded by GP49B. In some embodiments, the inhibitory receptor effector domain binds and modulates the activity of the inhibitory receptor encoded by Ly49Q. In some embodiments, the inhibitory receptor effector domain binds and modulates the activity of the inhibitory receptor encoded by MICL. In some embodiments, the inhibitory receptor effector domain binds and modulates the activity of the inhibitory receptor encoded by LAG3. In some embodiments, the inhibitory receptor effector domain binds and modulates the activity of the inhibitory receptor encoded by CD160. In some embodiments, the inhibitory receptor effector domain binds and modulates the activity of the inhibitory receptor encoded by FCRL4. In some embodiments, the inhibitory receptor effector domain binds and modulates the activity of the inhibitory receptor encoded by KIR3DL1. In some embodiments, the inhibitory receptor effector domain binds and modulates the activity of the inhibitory receptor encoded by KIR2DL2. In some embodiments, the inhibitory receptor effector domain binds and modulates the activity of the inhibitory receptor encoded by LILRB3. In some embodiments, the inhibitory receptor effector domain binds and modulates the activity of the inhibitory receptor encoded by DCIR. In some embodiments, the inhibitory receptor effector domain binds and modulates the activity of the inhibitory receptor encoded by NKRP-1D. In some embodiments, the inhibitory receptor effector domain binds and modulates the activity of the inhibitory receptor encoded by LY49. In some embodiments, the inhibitory receptor effector domain binds and modulates the activity of the inhibitory receptor encoded by MAIR-I. In some embodiments, the inhibitory receptor effector domain binds and modulates the activity of the inhibitory receptor encoded by CD79a. In some embodiments, the inhibitory receptor effector domain binds and modulates the activity of the inhibitory receptor encoded by CD79b. In some embodiments, the inhibitory receptor effector domain binds and modulates the activity of the inhibitory receptor encoded by CD19. In some embodiments, the inhibitory receptor effector domain binds and modulates the activity of the inhibitory receptor encoded by CD21. In some embodiments, the inhibitory receptor effector domain binds and modulates the activity of the inhibitory receptor encoded by CD40. In some embodiments, the inhibitory receptor effector domain binds and modulates the activity of the inhibitory receptor encoded by TLR3. In some embodiments, the inhibitory receptor effector domain binds and modulates the activity of the inhibitory receptor encoded by CD28. In some embodiments, the inhibitory receptor effector domain binds and modulates the activity of the inhibitory receptor encoded by CCR5. In some embodiments, the inhibitory receptor effector domain binds and modulates the activity of the inhibitory receptor encoded by CCR1.

In some embodiments, polypeptide comprises an inhibitory receptor effector domain that binds to LAG3, PDCD1, BTLA/CD272, CD200R1, CD22/Siglec2, CD300A, CD300LF/CD300F, CD33/Siglec3, CD5, CD72, CEACAM1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLEC5, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, 3, 4, 5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CD5, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1. In some embodiments, polypeptide comprises an inhibitory receptor effector domain that binds to PD-1. In some embodiments, polypeptide comprises an inhibitory receptor effector domain that binds to LAG-3. In some embodiments, polypeptide comprises an inhibitory receptor effector domain that binds to BTLA/CD272. In some embodiments, polypeptide comprises an inhibitory receptor effector domain that binds to CD200R1. In some embodiments, polypeptide comprises an inhibitory receptor effector domain that binds to CD22/Siglec2. In some embodiments, polypeptide comprises an inhibitory receptor effector domain that binds to CD300A. In some embodiments, polypeptide comprises an inhibitory receptor effector domain that binds to CD300LF/CD300F. In some embodiments, polypeptide comprises an inhibitory receptor effector domain that binds to CD33/Siglec3. In some embodiments, polypeptide comprises an inhibitory receptor effector domain that binds to CD5. In some embodiments, polypeptide comprises an inhibitory receptor effector domain that binds to CD72. In some embodiments, polypeptide comprises an inhibitory receptor effector domain that binds to CEACAM 1. In some embodiments, polypeptide comprises an inhibitory receptor effector domain that binds to CLEC12A. In some embodiments, polypeptide comprises an inhibitory receptor effector domain that binds to CLEC4A. In some embodiments, polypeptide comprises an inhibitory receptor effector domain that binds to CTLA4/CD152. In some embodiments, polypeptide comprises an inhibitory receptor effector domain that binds to FCGR2B/CD32B. In some embodiments, polypeptide comprises an inhibitory receptor effector domain that binds to KIRs. In some embodiments, polypeptide comprises an inhibitory receptor effector domain that binds to KLRB1/CD161. In some embodiments, polypeptide comprises an inhibitory receptor effector domain that binds to KLRC1. In some embodiments, polypeptide comprises an inhibitory receptor effector domain that binds to KLRG1. In some embodiments, polypeptide comprises an inhibitory receptor effector domain that binds to LAIR1. In some embodiments, polypeptide comprises an inhibitory receptor effector domain that binds to LILRB1. In some embodiments, polypeptide comprises an inhibitory receptor effector domain that binds to LILRB2. In some embodiments, polypeptide comprises an inhibitory receptor effector domain that binds to LILRB4. In some embodiments, polypeptide comprises an inhibitory receptor effector domain that binds to LILRB5. In some embodiments, polypeptide comprises an inhibitory receptor effector domain that binds to NCR2/NKp44. In some embodiments, polypeptide comprises an inhibitory receptor effector domain that binds to PDCD1. In some embodiments, polypeptide comprises an inhibitory receptor effector domain that binds to PECAM1/CD31. In some embodiments, polypeptide comprises an inhibitory receptor effector domain that binds to PILRA. In some embodiments, polypeptide comprises an inhibitory receptor effector domain that binds to PVR/CD155. In some embodiments, polypeptide comprises an inhibitory receptor effector domain that binds to SIGLEC11. In some embodiments, polypeptide comprises an inhibitory receptor effector domain that binds to SIGLECS. In some embodiments, polypeptide comprises an inhibitory receptor effector domain that binds to SIGLEC7. In some embodiments, polypeptide comprises an inhibitory receptor effector domain that binds to SIGLEC8. In some embodiments, polypeptide comprises an inhibitory receptor effector domain that binds to SIGLEC9. In some embodiments, polypeptide comprises an inhibitory receptor effector domain that binds to SIRPA. In some embodiments, polypeptide comprises an inhibitory receptor effector domain that binds to TIGIT. In some embodiments, polypeptide comprises an inhibitory receptor effector domain that binds to VSTM1/SIRL1. In some embodiments, polypeptide comprises an inhibitory receptor effector domain that binds to MAFA. In some embodiments, polypeptide comprises an inhibitory receptor effector domain that binds to NKG2A. In some embodiments, polypeptide comprises an inhibitory receptor effector domain that binds to CMRF35H. In some embodiments, polypeptide comprises an inhibitory receptor effector domain that binds to CD66a. In some embodiments, polypeptide comprises an inhibitory receptor effector domain that binds to CD66d. In some embodiments, polypeptide comprises an inhibitory receptor effector domain that binds to CD33. In some embodiments, polypeptide comprises an inhibitory receptor effector domain that binds to SIGLEC6. In some embodiments, polypeptide comprises an inhibitory receptor effector domain that binds to ILT2. In some embodiments, polypeptide comprises an inhibitory receptor effector domain that binds to ILT3. In some embodiments, polypeptide comprises an inhibitory receptor effector domain that binds to ILT4. In some embodiments, polypeptide comprises an inhibitory receptor effector domain that binds to ILT5. In some embodiments, polypeptide comprises an inhibitory receptor effector domain that binds to LIRE. In some embodiments, polypeptide comprises an inhibitory receptor effector domain that binds to KIR2DL. In some embodiments, polypeptide comprises an inhibitory receptor effector domain that binds to KIR3DL. In some embodiments, polypeptide comprises an inhibitory receptor effector domain that binds to SIRPa. In some embodiments, polypeptide comprises an inhibitory receptor effector domain that binds to KIR2DL2/3. In some embodiments, polypeptide comprises an inhibitory receptor effector domain that binds to KIR2DL5. In some embodiments, polypeptide comprises an inhibitory receptor effector domain that binds to KIRDL1. In some embodiments, polypeptide comprises an inhibitory receptor effector domain that binds to KIRDL2. In some embodiments, polypeptide comprises an inhibitory receptor effector domain that binds to KIRDL3. In some embodiments, polypeptide comprises an inhibitory receptor effector domain that binds to TIM3. In some embodiments, polypeptide comprises an inhibitory receptor effector domain that binds to Tactile. In some embodiments, polypeptide comprises an inhibitory receptor effector domain that binds to IRp60. In some embodiments, polypeptide comprises an inhibitory receptor effector domain that binds to NKRP1. In some embodiments, polypeptide comprises an inhibitory receptor effector domain that binds to IAP. In some embodiments, polypeptide comprises an inhibitory receptor effector domain that binds to PIR-B. In some embodiments, polypeptide comprises an inhibitory receptor effector domain that binds to 2B4. In some embodiments, polypeptide comprises an inhibitory receptor effector domain that binds to GP49B. In some embodiments, polypeptide comprises an inhibitory receptor effector domain that binds to Ly49Q. In some embodiments, polypeptide comprises an inhibitory receptor effector domain that binds to MICL. In some embodiments, polypeptide comprises an inhibitory receptor effector domain that binds to CD160. In some embodiments, polypeptide comprises an inhibitory receptor effector domain that binds to FCRL4. In some embodiments, polypeptide comprises an inhibitory receptor effector domain that binds to KIR3DL1. In some embodiments, polypeptide comprises an inhibitory receptor effector domain that binds to KIR2DL2. In some embodiments, polypeptide comprises an inhibitory receptor effector domain that binds to LILRB3. In some embodiments, polypeptide comprises an inhibitory receptor effector domain that binds to DCIR. In some embodiments, polypeptide comprises an inhibitory receptor effector domain that binds to NKRP-1D. In some embodiments, polypeptide comprises an inhibitory receptor effector domain that binds to LY49. In some embodiments, polypeptide comprises an inhibitory receptor effector domain that binds to MAIR-I. In some embodiments, polypeptide comprises an inhibitory receptor effector domain that binds to CD79a. In some embodiments, polypeptide comprises an inhibitory receptor effector domain that binds to CD79b. In some embodiments, polypeptide comprises an inhibitory receptor effector domain that binds to CD19. In some embodiments, polypeptide comprises an inhibitory receptor effector domain that binds to CD21. In some embodiments, polypeptide comprises an inhibitory receptor effector domain that binds to CD40. In some embodiments, polypeptide comprises an inhibitory receptor effector domain that binds to TLR3. In some embodiments, polypeptide comprises an inhibitory receptor effector domain that binds to CD28. In some embodiments, polypeptide comprises an inhibitory receptor effector domain that binds to CCR5. In some embodiments, polypeptide comprises an inhibitory receptor effector domain that binds to CCR1.

In some embodiments, polypeptide comprises an inhibitory receptor effector domain that binds to any one of LAG3, PDCD1, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLECS, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, 3, 4, 5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CDS, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1; and a second inhibitory receptor that binds to any one of LAG3, PDCD1, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLEC5, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, 3, 4, 5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CD5, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.

In some embodiments, polypeptide comprises an inhibitory receptor effector domain that binds to PD-1 and a second inhibitory receptor that binds to LAG-3. In some embodiments, polypeptide comprises an inhibitory receptor effector domain that binds to LAG-3 and a second inhibitory receptor that binds to PD-1.

In some embodiments, the polypeptide comprises an Fc domain as an effector domain to modulate the subject's response to the polypeptides, which can comprise a bifunctional antibody (two antigen binding domains that bind to the same or different targets as provided for herein). In some embodiments, the Fc domain comprises a mutation that selectively binds to FcγRIIb. In some embodiments, the Fc domain comprises a mutation that selectively binds to FcγRIIb over FcγRIIa. As used herein, in reference to FcγRIIb, the term “selectively binds to” means that the Fc domain binds preferentially to FcγRIIb, that is with a higher affinity to FcγRIIb. Examples of such mutations are provided for in, for example, U.S. Pat. Nos. 7,662,926, 7,655,229, US 2009/0087428, US 2007/0253948, and US 2006/0073142, each of which is hereby incorporated by reference in its entirety, including the specific mutations that are descried that affect the FcγRIIb or FcγRIIa binding. In some embodiments, the mutation is as described in Shields et al., J. Biol. Chem. 2001, 276:6591-6604, which is hereby incorporated by reference in its entirety, including the specific mutations that are described and that affect the FcγRIIb or FcγRIIa binding. In some embodiments, the mutations in the Fc region are at positions S298, E333, or K334, or any combination thereof (numbering according to EU numbering). In some embodiments, the Fc region comprises a mutation that corresponds to S298A, E333A, or K334A, or any combination thereof. In some embodiments, the Fc region comprises the mutations of S298A, E333A, and K334A. In some embodiments, the mutations correspond to G236A, I332E, G236A, S239D, or I332E, or any combination thereof. In some embodiments, the Fc region comprises the mutations of G236A, I332E, G236A, S239D, and I332E. The mutations can also be as provided for in, Richards et al., Mol Cancer Ther 2008; 7(8). August 2008, which is hereby incorporated by reference in its entirety, including the specific mutations that are descried that affect the FcγRIIb or FcγRIIa binding. In some embodiments, the Fc region comprises a N235S or L328F mutation. In some embodiments, the Fc region comprises a N235S and L328F mutation. The mutations can also be as provided for in Shang et al., The Journal of Biological Chemistry VOL. 289, NO. 22, pp. 15309-15318, May 30, 2014, which is hereby incorporated by reference in its entirety, including the specific mutations that are descried that affect the FcγRIIb or FcγRIIa binding.

In some embodiments, the Fc mutation is as described in U.S. Pat. No. 10,618,965; EP Serial No. 2679681; EP Serial No. 3604330, US 2014/0093496, US 2015/0203577, U.S. Pat. No. 9,540,451, EP Serial No. 2331578; EP Serial No. 3190128; U.S. Pat. No. 9,902,773; EP. Serial No. 3342782, U.S. Publication No. 2020/0332024; EP Serial No. 2796469; EP Serial No. 2331578; EP Serial No. 3190128; U.S. Pat. No. 9,902,773, EP Serial No. 2331578; EP Serial No. 3190128, U.S. Pat. Nos. 9,493,578, 9,394,366, 9,914,778, EP Serial No. 2940043, U.S. Pat. No. 9,890,218, EP Serial No. 2940135; U.S. Pat. Nos. 10,766,960, 10,919,953, EP 3721900, EP2889377, US 2016/0039912, EP 2982689, or EP 3783017, each of which is hereby incorporated by reference in its entirety, including the specific mutations that are descried that affect the FcγRIIb or FcγRIIa binding.

In some embodiments, the Fc region comprises a mutation, mutations, or a mutation set that increases selectivity for FcγRIIb. In some embodiments, the Fc region comprises a mutation, mutations, or a mutation set that increases affinity for FcγRIIb. In some embodiments, the Fc region comprises a mutation, mutations, or a mutation set that increases selectivity and affinity for FcγRIIb. In some embodiments, the Fc region comprises a mutation, mutations, or a mutation set that increases selectivity for FcγRIIb over FcγRIIa. In some embodiments, the Fc region comprises a mutation, mutations, or a mutation set that increases affinity for FcγRIIb over FcγRIIa. In some embodiments, the Fc region comprises a mutation, mutations, or a mutation set that increases selectivity and affinity for FcγRIIb over FcγRIIa. In some embodiments, the mutation, mutations, or the mutation set is such as those described herein.

In some embodiments, the Fc region comprises a mutation, mutations, or a mutation set, of P238D; P238D and E233D; P238D and L234W; P238D and L234Y; P238D and G237W; P238D and G237F; P238D and G237A; P238D and G237D; P238D and G237E; P238D and G237L; P238D and G237M; P238D and G237Y; P238D and S239D; P238D and S267V; P238D and S267Q; P238D and S267A; P238D and H268N; P238D and H268D; P238D and H268E; P238D and P271G; P238D and Y296D; P238D and V323I; P238D and V323L; P238D and V323M; P238D and K326L; P238D and K326Q; P238D and K326E; P238D and K326M; P238D and K326D; P238D and K326S; P238D and K326T; P238D and K326A; P238D and K326N; P238D and L328E; P238D and A330K; P238D and A330R; P238D and A330M; S239P; S239P and P230E; S239P and A231D; S239P and P232E; S239P and P238E; S239P, P230E and A231D; S239P, P230E and P232E; S239P, P230E and P238E; S239P, P230E, A231D and P232E; S239P, P230E, A231D and P238E; S239P, P230E, A231D, P232E and P238E; S239P, A231D and P232E; S239P, A231D and P238E; S239P, A231D, P232E and P238E; S239P, P232E and P238E; S267E; S267D; S267E and L328F; G236D and S267E; S239D and S267E; S239D and I332E; K409E; L368K; S364D and K370G; S364Y and K370R; S364D; Y349K; K409D; K392E; D399K; S364E; L368E and K409E; S364E and F405A; Y349K and T394F; S364H and Y349K; P395T, V397S and F405A; T394F; T394S, P395V, P396T, V397E and F405S; V397S and F405A; S364H, D401K and F405A; Y349T, T394F and T411E; L351K, S364H and D401K; Y349T, L351E and T411E; S364H; Y349T; S364H and D401K; Y349T and T411E; S364H and T394F; Y349T and F405A; S364H and F405A; Y349T and T394F; F405A; S364E and T394F; Y349K and F405A; V397T and F405S; S364E and F405S; Y349K and T394Y; S364E, T411E and F405A; Y349K, T394F and D401K; S364E and T411E; Y349K and D401K; L351E and S364D; Y349K and L351K; L351E and S364E; Y349C and S364E; Y349K and S354C; S364H, F405A and T411E; Y349T, T394F and D401K; S364D and T394F; L235Y; L235R; G236D; L328F; L235Y, G236D, S267D and L328F; L235Y, G236D and S267D; L235Y, G236D and S267E; L235Y and G236D; L235Y, S267D and L328F; L235Y, S267E and L328F; L235Y and L328F; L235R, G236D, S267D and L328F; L235R, G236D and S267D; L235R, G236D and S267E; L235R and G236D; L235R, S267D and L328F; L235R, S267E and L328F; L235R and L328F; G236D, S267E and L328F; G236D, S267D and L328F; G236D and L328F; S267D and L328F; G236N and S267E; G236N; L234Y, L235Y, G236W, H268D and S298A; L234Y, L235Y, G236W, H268D, D270E and S298A; L234Y, L235Q, G236W, S239M, H268D, D270E and S298A; L234Y, L235Y, G236W, H268D, S298A and A327D; L234Y, L235Y, G236W, S239M, H268D, S298A and A327D; L234Y, L235Y, G236W, S239M, H268D, S298A, A327D, L328W and K334L; second IgG1 CH2 Domain; K326D, A330M and K334E; D270E, K326D, A330M and K334E; D270E, K326D, A330K and K334E; L234E, L235Y, G236W, S239M, H268D, S298A and A327D; L234S, L235Y, G236W, S239M, H268D, S298A and A327D; L235Q, G236W, S239M, H268D, D270E and S298A; L235Y, G236W, S239M, H268D, S298A and A327D; L234S, L235Q, G236W, S239M, H268D, D270E and S298A; L234F, L235Q, G236W, S239M, H268D, D270E and S298A; L234E, L235Q, G236W, S239M, H268D, D270E and S298A; L234F, L235Y, G236W, S239M, H268D, S298A and A327D; L234V, L235Q, G236W, S239M, H268D, D270E and S298A; L234D, L235Q, G236W, S239M, H268D, D270E and S298A; L234Q, L235Q, G236W, S239M, H268D, D270E and S298A; L234I, L235Q, G236W, S239M, H268D, D270E and S298A; L234M, L235Q, G236W, S239M, H268D, D270E and S298A; L234T, L235Q, G236W, S239M, H268D, D270E and S298A; L234A, L235Q, G236W, S239M, H268D, D270E and S298A; L234G, L235Q, G236W, S239M, H268D, D270E and S298A; L234H, L235Q, G236W, S239M, H268D, D270E and S298A; L234V, L235Y, G236W, S239M, H268D, S298A and A327D; L234D, L235Y, G236W, S239M, H268D, S298A and A327D; L234Q, L235Y, G236W, S239M, H268D, S298A and A327D; L234I, L235Y, G236W, S239M, H268D, S298A and A327D; L234M, L235Y, G236W, S239M, H268D, S298A and A327D; L234T, L235Y, G236W, S239M, H268D, S298A and A327D; L234A, L235Y, G236W, S239M, H268D, S298A and A327D; L234G, L235Y, G236W, S239M, H268D, S298A and A327D; L234H, L235Y, G236W, S239M, H268D, S298A and A327D; L234F, L235Q, G236W, S239I, H268D, D270E and S298A; L234E, L235Q, G236W, S239I, H268D, D270E and S298A; L234D, L235Q, G236W, S239I, H268D, D270E and S298A; L234V, L235Y, G236W, S239I, H268D, S298A and A327D; L234I and L235Y, G236W, S239I, H268D, S298A, A327D; L235Y, G236W, S239I, H268D, S298A, A327D; L234E, L235Y, G236W, S239I, H268D, S298A and A327D; L234D, L235Y, G236W, S239I, H268D, S298A and A327D; L234F, L235Y, G236W, S239I, H268D, S298A and A327D; L234T, L235Y, G236W, S239I, H268D, S298A and A327D; second polypeptide; D270E, K326D and K334E; D270E, K326D, A330F and K334E; D270E, K326D, A330I and K334E; D270E, K326D, A330Y and K334E; D270E, K326D, A330H and K334E; P238D, E233D, G237D, H268D, P271G, Y296D and A330R; P238D, G237D, H268D, P271G, Y296D and A330R; P238D, G237D, H268E, P271G, Y296D and A330R; P238D, E233D, G237D, H268D, P271G, Y296D, A330R and I332T; P238D, E233D, G237D, V264I, S267G, H268E, P271G and A330R; P238D, E233D, G237D, V264I, S267A, H268E, P271G and A330R; P238D, E233D, G237D, S267A, H268E, P271G, Y296D, A330R and I332T; P238D, G237D, S267A, H268E, P271G, Y296D, A330R and I332T; P238D, E233D, G237D, V264I, S267A, H268E and P271G; P238D, E233D, G237D, V264I, S267A, H268E, P271G, Y296D and A330R; P238D, E233D, G237D, V264I, S267A, H268E, P271G, Y296D, A330R and P396M; P238D, E233D, G237D, V264I, S267A, H268E, P271G, Y296D, A330R and P396L; P238D, G237D, V264I, S267A, H268E, P271G and A330R; P238D, G237D, V264I, S267A, H268E, P271G, Y296D and A330R; P238D, V264I, S267A, H268E and P271G; P238D, V264I, S267A, H268E, P271G and Y296D; P238D, G237D, S267A, H268E, P271G, Y296D and A330R; P238D, G237D, S267G, H268E, P271G, Y296D and A330R; P238D, E233D, G237D, V264I, S267A, H268E, P271G, A330R and P396M; P238D, E233D, G237D, V264I, S267A, H268E, P271G, A330R and P396L; P238D, E233D, G237D, V264I, S267A, H268E, P271G, Y296D, A327G, A330R and P396M; P238D, E233D, G237D, V264I, S267A, H268E, P271G, E272D and Y296D; P238D, G237D, V264I, S267A, H268E, P271G, E272P and A330R; P238D, G237D, V264I, S267A, H268E, P271G, E272P, Y296D and A330R; P238D, E233D, V264I, S267A, H268E and P271G; P238D, G237D, S267E, H268D, P271G, Y296D and A330R; P238D, V264I, S267A, H268E, P271G, E272D and Y296D; P238D, E233D, V264I, S267A, H268E, P271G and Y296D; P238D, E233D, L234Y, L235F, G237D, V264I, D265E, V266F, S267A, H268D, E269D, P271G, E272D, K274Q, Y296D, K326A, A327G, A330K, P331S, I332K, E333K, K334R, R355A, D356E, L358M, P396A, K409R and Q419E; G237Q, P238D, F241M, Y296E, A330H and S324H; G237Q, P238D, F241M, H268P, Y296E and A330H; G237Q, P238D, L235F, F241M, Y296E and S324H; G237Q, P238D, L235F, F241M, H268P and Y296E; G237Q, P238D, F241M, H268P, Y296E and S324H; G237Q, P238D, L235F, F241M, H268P, Y296E and S324H; G237Q, P238D, L235F, F241M, Y296E, S324H and A330H; G237Q, P238D, L235F, F241M, H268P, Y296E and A330H; G237Q, P238D, F241M, H268P, Y296E, S324H and A330H; G237Q, P238D, E233D, V264I, S267R, H268P, P271G and Y296E; G237Q, P238D, F241M and Y296E; G237Q, P238D, F241M, Y296E and A330H; G237Q, P238D, L235F, F241M and Y296E; G237Q, P238D, L235F, F241M, Y296E and A330H; G237Q and P238D; P238D and F241M; P238D and F241L; P238D and H268P; P238D and Q295V; P238D and Y296E; P238D and Y296H; P238D and S298M; P238D and S324N; P238D and S324H; P238D and A330H; P238D and A330Y; P238D and F241M, H268P, Y296E and S324H; G237Q, P238D, F241M, Y296E and A330H; L235F, G237Q, P238D, F241M and Y296E; P238D, P271G and E233D; P238D, P271G and L234R; P238D, P271G and G237D; P238D, P271G and G237K; P238D, P271G and V264I; P238D, P271G and S267A; P238D, P271G and H268E; P238D, P271G and H268P; P238D, P271G and Y296D; P238D, P271G and Y296E; P238D, P271G, E233D, L234K, V264I, S267A and H268E; P238D, P271G, E233D, L234R, V264I, S267A and H268E; P238D, P271G, E233D, G237K, V264I, S267A and H268E; P238D, P271G, E233D, V264I, D265N, S267A and H268E; P238D, P271G, E233D, V264I, S267R and H268E; P238D, P271G, E233D, G237D, V264I, S267Y, H268E, Y296D, A330R and P396M; P238D, P271G, E233D, G237D, V264I, S267A, H268E, Y296D/Y296A, A330R and P396M; P238D, P271G, E233D, V264I, S267R, H268E and Y296E; P238D, P271G, E233D, V264I, S267R and H268P; P238D, P271G, E233D, F241M, V264I, S267R and H268E; P238D, P271G, E233D, V264I, S267R, H268P and Y296E; P238D, P271G, E233D, G237Q, V264I, S267R, H268P and Y296E; E233D, G237D, P238D, H268D, P271G, and A330R.

In some embodiments, the Fc region comprises a mutation of P238D. In some embodiments, the Fc region comprises a mutation or mutations of P238D and E233D. In some embodiments, the Fc region comprises a mutation or mutations of P238D and L234W. In some embodiments, the Fc region comprises a mutation or mutations of P238D and L234Y. In some embodiments, the Fc region comprises a mutation or mutations of P238D and G237W. In some embodiments, the Fc region comprises a mutation or mutations of P238D and G237F. In some embodiments, the Fc region comprises a mutation or mutations of P238D and G237A. In some embodiments, the Fc region comprises a mutation or mutations of P238D and G237D. In some embodiments, the Fc region comprises a mutation or mutations of P238D and G237E. In some embodiments, the Fc region comprises a mutation or mutations of P238D and G237L. In some embodiments, the Fc region comprises a mutation or mutations of P238D and G237M. In some embodiments, the Fc region comprises a mutation or mutations of P238D and G237Y. In some embodiments, the Fc region comprises a mutation or mutations of P238D and S239D. In some embodiments, the Fc region comprises a mutation or mutations of P238D and S267V. In some embodiments, the Fc region comprises a mutation or mutations of P238D and S267Q. In some embodiments, the Fc region comprises a mutation or mutations of P238D and S267A. In some embodiments, the Fc region comprises a mutation or mutations of P238D and H268N. In some embodiments, the Fc region comprises a mutation or mutations of P238D and H268D. In some embodiments, the Fc region comprises a mutation or mutations of P238D and H268E. In some embodiments, the Fc region comprises a mutation or mutations of P238D and P271G. In some embodiments, the Fc region comprises a mutation or mutations of P238D and Y296D. In some embodiments, the Fc region comprises a mutation or mutations of P238D and V323I. In some embodiments, the Fc region comprises a mutation or mutations of P238D and V323L. In some embodiments, the Fc region comprises a mutation or mutations of P238D and V323M. In some embodiments, the Fc region comprises a mutation or mutations of P238D and K326L. In some embodiments, the Fc region comprises a mutation or mutations of P238D and K326Q. In some embodiments, the Fc region comprises a mutation or mutations of P238D and K326E. In some embodiments, the Fc region comprises a mutation or mutations of P238D and K326M. In some embodiments, the Fc region comprises a mutation or mutations of P238D and K326D. In some embodiments, the Fc region comprises a mutation or mutations of P238D and K326S. In some embodiments, the Fc region comprises a mutation or mutations of P238D and K326T. In some embodiments, the Fc region comprises a mutation or mutations of P238D and K326A. In some embodiments, the Fc region comprises a mutation or mutations of P238D and K326N. In some embodiments, the Fc region comprises a mutation or mutations of P238D and L328E. In some embodiments, the Fc region comprises a mutation or mutations of P238D and A330K. In some embodiments, the Fc region comprises a mutation or mutations of P238D and A330R. In some embodiments, the Fc region comprises a mutation or mutations of P238D and A330M. In some embodiments, the Fc region comprises a mutation of S239P. In some embodiments, the Fc region comprises a mutation or mutations of S239P and P230E. In some embodiments, the Fc region comprises a mutation or mutations of S239P and A231D. In some embodiments, the Fc region comprises a mutation or mutations of S239P and P232E. In some embodiments, the Fc region comprises a mutation or mutations of S239P and P238E. In some embodiments, the Fc region comprises a mutation or mutations of S239P, P230E and A231D. In some embodiments, the Fc region comprises a mutation or mutations of S239P, P230E and P232E. In some embodiments, the Fc region comprises a mutation or mutations of S239P, P230E and P238E. In some embodiments, the Fc region comprises a mutation or mutations of S239P, P230E, A231D and P232E. In some embodiments, the Fc region comprises a mutation or mutations of S239P, P230E, A231D and P238E. In some embodiments, the Fc region comprises a mutation or mutations of S239P, P230E, A231D, P232E and P238E. In some embodiments, the Fc region comprises a mutation or mutations of S239P, A231D and P232E. In some embodiments, the Fc region comprises a mutation or mutations of S239P, A231D and P238E. In some embodiments, the Fc region comprises a mutation or mutations of S239P, A231D, P232E and P238E. In some embodiments, the Fc region comprises a mutation or mutations of S239P, P232E and P238E. In some embodiments, the Fc region comprises a mutation of S267E. In some embodiments, the Fc region comprises a mutation of S267D. In some embodiments, the Fc region comprises a mutation or mutations of S267E and L328F. In some embodiments, the Fc region comprises a mutation or mutations of G236D and S267E. In some embodiments, the Fc region comprises a mutation or mutations of S239D and S267E. In some embodiments, the Fc region comprises a mutation or mutations of S239D and I332E. In some embodiments, the Fc region comprises a mutation of K409E. In some embodiments, the Fc region comprises a mutation of L368K. In some embodiments, the Fc region comprises a mutation or mutations of S364D and K370G. In some embodiments, the Fc region comprises a mutation or mutations of S364Y and K370R. In some embodiments, the Fc region comprises a mutation of S364D. In some embodiments, the Fc region comprises a mutation of Y349K. In some embodiments, the Fc region comprises a mutation of K409D. In some embodiments, the Fc region comprises a mutation of K392E. In some embodiments, the Fc region comprises a mutation of D399K. In some embodiments, the Fc region comprises a mutation of S364E. In some embodiments, the Fc region comprises a mutation or mutations of L368E and K409E. In some embodiments, the Fc region comprises a mutation or mutations of S364E and F405A. In some embodiments, the Fc region comprises a mutation or mutations of Y349K and T394F. In some embodiments, the Fc region comprises a mutation or mutations of S364H and Y349K. In some embodiments, the Fc region comprises a mutation or mutations of P395T, V397S and F405A. In some embodiments, the Fc region comprises a mutation of T394F. In some embodiments, the Fc region comprises a mutation or mutations of T394S, P395V, P396T, V397E and F405S. In some embodiments, the Fc region comprises a mutation or mutations of V397S and F405A. In some embodiments, the Fc region comprises a mutation or mutations of S364H, D401K and F405A. In some embodiments, the Fc region comprises a mutation or mutations of Y349T, T394F and T411E. In some embodiments, the Fc region comprises a mutation or mutations of L351K, S364H and D401K. In some embodiments, the Fc region comprises a mutation or mutations of Y349T, L351E and T411E. In some embodiments, the Fc region comprises a mutation of S364H. In some embodiments, the Fc region comprises a mutation of Y349T. In some embodiments, the Fc region comprises a mutation or mutations of S364H and D401K. In some embodiments, the Fc region comprises a mutation or mutations of Y349T and T411E. In some embodiments, the Fc region comprises a mutation or mutations of S364H and T394F. In some embodiments, the Fc region comprises a mutation or mutations of Y349T and F405A. In some embodiments, the Fc region comprises a mutation or mutations of S364H and F405A. In some embodiments, the Fc region comprises a mutation or mutations of Y349T and T394F. In some embodiments, the Fc region comprises a mutation of F405A. In some embodiments, the Fc region comprises a mutation or mutations of S364E and T394F. In some embodiments, the Fc region comprises a mutation or mutations of Y349K and F405A. In some embodiments, the Fc region comprises a mutation or mutations of V397T and F405S. In some embodiments, the Fc region comprises a mutation or mutations of S364E and F405S. In some embodiments, the Fc region comprises a mutation or mutations of Y349K and T394Y. In some embodiments, the Fc region comprises a mutation or mutations of S364E, T411E and F405A. In some embodiments, the Fc region comprises a mutation or mutations of Y349K, T394F and D401K. In some embodiments, the Fc region comprises a mutation or mutations of S364E and T411E. In some embodiments, the Fc region comprises a mutation or mutations of Y349K and D401K. In some embodiments, the Fc region comprises a mutation or mutations of L351E and S364D. In some embodiments, the Fc region comprises a mutation or mutations of Y349K and L351K. In some embodiments, the Fc region comprises a mutation or mutations of L351E and S364E. In some embodiments, the Fc region comprises a mutation or mutations of Y349C and S364E. In some embodiments, the Fc region comprises a mutation or mutations of Y349K and S354C. In some embodiments, the Fc region comprises a mutation or mutations of S364H, F405A and T411E. In some embodiments, the Fc region comprises a mutation or mutations of Y349T, T394F and D401K. In some embodiments, the Fc region comprises a mutation or mutations of S364D and T394F. In some embodiments, the Fc region comprises a mutation of L235Y. In some embodiments, the Fc region comprises a mutation of L235R. In some embodiments, the Fc region comprises a mutation of G236D. In some embodiments, the Fc region comprises a mutation of L328F. In some embodiments, the Fc region comprises a mutation or mutations of L235Y, G236D, S267D and L328F. In some embodiments, the Fc region comprises a mutation or mutations of L235Y, G236D and S267D. In some embodiments, the Fc region comprises a mutation or mutations of L235Y, G236D and S267E. In some embodiments, the Fc region comprises a mutation or mutations of L235Y and G236D. In some embodiments, the Fc region comprises a mutation or mutations of L235Y, S267D and L328F. In some embodiments, the Fc region comprises a mutation or mutations of L235Y, S267E and L328F. In some embodiments, the Fc region comprises a mutation or mutations of L235Y and L328F. In some embodiments, the Fc region comprises a mutation or mutations of L235R, G236D, S267D and L328F. In some embodiments, the Fc region comprises a mutation or mutations of L235R, G236D and S267D. In some embodiments, the Fc region comprises a mutation or mutations of L235R, G236D and S267E. In some embodiments, the Fc region comprises a mutation or mutations of L235R and G236D. In some embodiments, the Fc region comprises a mutation or mutations of L235R, S267D and L328F. In some embodiments, the Fc region comprises a mutation or mutations of L235R, S267E and L328F. In some embodiments, the Fc region comprises a mutation or mutations of L235R and L328F. In some embodiments, the Fc region comprises a mutation or mutations of G236D, S267E and L328F. In some embodiments, the Fc region comprises a mutation or mutations of G236D, S267D and L328F. In some embodiments, the Fc region comprises a mutation or mutations of G236D and L328F. In some embodiments, the Fc region comprises a mutation or mutations of S267D and L328F. In some embodiments, the Fc region comprises a mutation or mutations of G236N and S267E. In some embodiments, the Fc region comprises a mutation of G236N.

In some embodiments, the Fc region comprises a mutation or mutations of L234Y, L235Y, G236W, H268D and S298A. In some embodiments, the Fc region comprises a mutation or mutations of L234Y, L235Y, G236W, H268D, D270E and S298A. In some embodiments, the Fc region comprises a mutation or mutations of L234Y, L235Q, G236W, S239M, H268D, D270E and S298A. In some embodiments, the Fc region comprises a mutation or mutations of L234Y, L235Y, G236W, H268D, S298A and A327D. In some embodiments, the Fc region comprises a mutation or mutations of L234Y, L235Y, G236W, S239M, H268D, S298A and A327D. In some embodiments, the Fc region comprises a mutation or mutations of L234Y, L235Y, G236W, S239M, H268D, S298A, A327D, L328W and K334L. In some embodiments, the Fc region comprises a mutation or mutations of second IgG1 CH2 Domain. In some embodiments, the Fc region comprises a mutation or mutations of K326D, A330M and K334E. In some embodiments, the Fc region comprises a mutation or mutations of D270E, K326D, A330M and K334E. In some embodiments, the Fc region comprises a mutation or mutations of D270E, K326D, A330K and K334E. In some embodiments, the Fc region comprises a mutation or mutations of L234E, L235Y, G236W, S239M, H268D, S298A and A327D. In some embodiments, the Fc region comprises a mutation or mutations of L234S, L235Y, G236W, S239M, H268D, S298A and A327D. In some embodiments, the Fc region comprises a mutation or mutations of L235Q, G236W, S239M, H268D, D270E and S298A. In some embodiments, the Fc region comprises a mutation or mutations of L235Y, G236W, S239M, H268D, S298A and A327D. In some embodiments, the Fc region comprises a mutation or mutations of L234S, L235Q, G236W, S239M, H268D, D270E and S298A. In some embodiments, the Fc region comprises a mutation or mutations of L234F, L235Q, G236W, S239M, H268D, D270E and S298A. In some embodiments, the Fc region comprises a mutation or mutations of L234E, L235Q, G236W, S239M, H268D, D270E and S298A. In some embodiments, the Fc region comprises a mutation or mutations of L234F, L235Y, G236W, S239M, H268D, S298A and A327D. In some embodiments, the Fc region comprises a mutation or mutations of L234V, L235Q, G236W, S239M, H268D, D270E and S298A. In some embodiments, the Fc region comprises a mutation or mutations of L234D, L235Q, G236W, S239M, H268D, D270E and S298A. In some embodiments, the Fc region comprises a mutation or mutations of L234Q, L235Q, G236W, S239M, H268D, D270E and S298A. In some embodiments, the Fc region comprises a mutation or mutations of L234I, L235Q, G236W, S239M, H268D, D270E and S298A. In some embodiments, the Fc region comprises a mutation or mutations of L234M, L235Q, G236W, S239M, H268D, D270E and S298A. In some embodiments, the Fc region comprises a mutation or mutations of L234T, L235Q, G236W, S239M, H268D, D270E and S298A. In some embodiments, the Fc region comprises a mutation or mutations of L234A, L235Q, G236W, S239M, H268D, D270E and S298A. In some embodiments, the Fc region comprises a mutation or mutations of L234G, L235Q, G236W, S239M, H268D, D270E and S298A. In some embodiments, the Fc region comprises a mutation or mutations of L234H, L235Q, G236W, S239M, H268D, D270E and S298A. In some embodiments, the Fc region comprises a mutation or mutations of L234V, L235Y, G236W, S239M, H268D, S298A and A327D. In some embodiments, the Fc region comprises a mutation or mutations of L234D, L235Y, G236W, S239M, H268D, S298A and A327D. In some embodiments, the Fc region comprises a mutation or mutations of L234Q, L235Y, G236W, S239M, H268D, S298A and A327D. In some embodiments, the Fc region comprises a mutation or mutations of L234I, L235Y, G236W, S239M, H268D, S298A and A327D. In some embodiments, the Fc region comprises a mutation or mutations of L234M, L235Y, G236W, S239M, H268D, S298A and A327D. In some embodiments, the Fc region comprises a mutation or mutations of L234T, L235Y, G236W, S239M, H268D, S298A and A327D. In some embodiments, the Fc region comprises a mutation or mutations of L234A, L235Y, G236W, S239M, H268D, S298A and A327D. In some embodiments, the Fc region comprises a mutation or mutations of L234G, L235Y, G236W, S239M, H268D, S298A and A327D. In some embodiments, the Fc region comprises a mutation or mutations of L234H, L235Y, G236W, S239M, H268D, S298A and A327D. In some embodiments, the Fc region comprises a mutation or mutations of L234F, L235Q, G236W, S239I, H268D, D270E and S298A. In some embodiments, the Fc region comprises a mutation or mutations of L234E, L235Q, G236W, S239I, H268D, D270E and S298A. In some embodiments, the Fc region comprises a mutation or mutations of L234D, L235Q, G236W, S239I, H268D, D270E and S298A. In some embodiments, the Fc region comprises a mutation or mutations of L234V, L235Y, G236W, S239I, H268D, S298A and A327D. In some embodiments, the Fc region comprises a mutation or mutations of L234I and L235Y, G236W, S239I, H268D, S298A, A327D. In some embodiments, the Fc region comprises a mutation or mutations of L235Y, G236W, S239I, H268D, S298A, A327D. In some embodiments, the Fc region comprises a mutation or mutations of L234E, L235Y, G236W, S239I, H268D, S298A and A327D. In some embodiments, the Fc region comprises a mutation or mutations of L234D, L235Y, G236W, S239I, H268D, S298A and A327D. In some embodiments, the Fc region comprises a mutation or mutations of L234F, L235Y, G236W, S239I, H268D, S298A and A327D. In some embodiments, the Fc region comprises a mutation or mutations of L234T, L235Y, G236W, S239I, H268D, S298A and A327D. In some embodiments, the Fc region comprises a mutation or mutations of second polypeptide. In some embodiments, the Fc region comprises a mutation or mutations of D270E, K326D and K334E. In some embodiments, the Fc region comprises a mutation or mutations of D270E, K326D, A330F and K334E. In some embodiments, the Fc region comprises a mutation or mutations of D270E, K326D, A330I and K334E. In some embodiments, the Fc region comprises a mutation or mutations of D270E, K326D, A330Y and K334E. In some embodiments, the Fc region comprises a mutation or mutations of D270E, K326D, A330H and K334E.

In some embodiments, the Fc region comprises a mutation or mutations of P238D, E233D, G237D, H268D, P271G, Y296D and A330R. In some embodiments, the Fc region comprises a mutation or mutations of P238D, G237D, H268D, P271G, Y296D and A330R. In some embodiments, the Fc region comprises a mutation or mutations of P238D, G237D, H268E, P271G, Y296D and A330R. In some embodiments, the Fc region comprises a mutation or mutations of P238D, E233D, G237D, H268D, P271G, Y296D, A330R and I332T. In some embodiments, the Fc region comprises a mutation or mutations of P238D, E233D, G237D, V264I, S267G, H268E, P271G and A330R. In some embodiments, the Fc region comprises a mutation or mutations of P238D, E233D, G237D, V264I, S267A, H268E, P271G and A330R. In some embodiments, the Fc region comprises a mutation or mutations of P238D, E233D, G237D, S267A, H268E, P271G, Y296D, A330R and I332T. In some embodiments, the Fc region comprises a mutation or mutations of P238D, G237D, S267A, H268E, P271G, Y296D, A330R and I332T. In some embodiments, the Fc region comprises a mutation or mutations of P238D, E233D, G237D, V264I, S267A, H268E and P271G. In some embodiments, the Fc region comprises a mutation or mutations of P238D, E233D, G237D, V264I, S267A, H268E, P271G, Y296D and A330R. In some embodiments, the Fc region comprises a mutation or mutations of P238D, E233D, G237D, V264I, S267A, H268E, P271G, Y296D, A330R and P396M. In some embodiments, the Fc region comprises a mutation or mutations of P238D, E233D, G237D, V264I, S267A, H268E, P271G, Y296D, A330R and P396L. In some embodiments, the Fc region comprises a mutation or mutations of P238D, G237D, V264I, S267A, H268E, P271G and A330R. In some embodiments, the Fc region comprises a mutation or mutations of P238D, G237D, V264I, S267A, H268E, P271G, Y296D and A330R. In some embodiments, the Fc region comprises a mutation or mutations of P238D, V264I, S267A, H268E and P271G. In some embodiments, the Fc region comprises a mutation or mutations of P238D, V264I, S267A, H268E, P271G and Y296D. In some embodiments, the Fc region comprises a mutation or mutations of P238D, G237D, S267A, H268E, P271G, Y296D and A330R. In some embodiments, the Fc region comprises a mutation or mutations of P238D, G237D, S267G, H268E, P271G, Y296D and A330R. In some embodiments, the Fc region comprises a mutation or mutations of P238D, E233D, G237D, V264I, S267A, H268E, P271G, A330R and P396M. In some embodiments, the Fc region comprises a mutation or mutations of P238D, E233D, G237D, V264I, S267A, H268E, P271G, A330R and P396L. In some embodiments, the Fc region comprises a mutation or mutations of P238D, E233D, G237D, V264I, S267A, H268E, P271G, Y296D, A327G, A330R and P396M. In some embodiments, the Fc region comprises a mutation or mutations of P238D, E233D, G237D, V264I, S267A, H268E, P271G, E272D and Y296D. In some embodiments, the Fc region comprises a mutation or mutations of P238D, G237D, V264I, S267A, H268E, P271G, E272P and A330R. In some embodiments, the Fc region comprises a mutation or mutations of P238D, G237D, V264I, S267A, H268E, P271G, E272P, Y296D and A330R. In some embodiments, the Fc region comprises a mutation or mutations of P238D, E233D, V264I, S267A, H268E and P271G. In some embodiments, the Fc region comprises a mutation or mutations of P238D, G237D, S267E, H268D, P271G, Y296D and A330R. In some embodiments, the Fc region comprises a mutation or mutations of P238D, V264I, S267A, H268E, P271G, E272D and Y296D. In some embodiments, the Fc region comprises a mutation or mutations of P238D, E233D, V264I, S267A, H268E, P271G and Y296D. In some embodiments, the Fc region comprises a mutation or mutations of P238D, E233D, L234Y, L235F, G237D, V264I, D265E, V266F, S267A, H268D, E269D, P271G, E272D, K274Q, Y296D, K326A, A327G, A330K, P331S, I332K, E333K, K334R, R355A, D356E, L358M, P396A, K409R and Q419E.

In some embodiments, the Fc region comprises a mutation or mutations of G237Q, P238D, F241M, Y296E, A330H and S324H. In some embodiments, the Fc region comprises a mutation or mutations of G237Q, P238D, F241M, H268P, Y296E and A330H. In some embodiments, the Fc region comprises a mutation or mutations of G237Q, P238D, L235F, F241M, Y296E and S324H. In some embodiments, the Fc region comprises a mutation or mutations of G237Q, P238D, L235F, F241M, H268P and Y296E. In some embodiments, the Fc region comprises a mutation or mutations of G237Q, P238D, F241M, H268P, Y296E and S324H. In some embodiments, the Fc region comprises a mutation or mutations of G237Q, P238D, L235F, F241M, H268P, Y296E and S324H. In some embodiments, the Fc region comprises a mutation or mutations of G237Q, P238D, L235F, F241M, Y296E, S324H and A330H. In some embodiments, the Fc region comprises a mutation or mutations of G237Q, P238D, L235F, F241M, H268P, Y296E and A330H. In some embodiments, the Fc region comprises a mutation or mutations of G237Q, P238D, F241M, H268P, Y296E, S324H and A330H. In some embodiments, the Fc region comprises a mutation or mutations of G237Q, P238D, E233D, V264I, S267R, H268P, P271G and Y296E. In some embodiments, the Fc region comprises a mutation or mutations of G237Q, P238D, F241M and Y296E. In some embodiments, the Fc region comprises a mutation or mutations of G237Q, P238D, F241M, Y296E and A330H. In some embodiments, the Fc region comprises a mutation or mutations of G237Q, P238D, L235F, F241M and Y296E. In some embodiments, the Fc region comprises a mutation or mutations of G237Q, P238D, L235F, F241M, Y296E and A330H.

In some embodiments, the Fc region comprises a mutation or mutations of G237Q and P238D. In some embodiments, the Fc region comprises a mutation or mutations of P238D and F241M. In some embodiments, the Fc region comprises a mutation or mutations of P238D and F241L. In some embodiments, the Fc region comprises a mutation or mutations of P238D and H268P. In some embodiments, the Fc region comprises a mutation or mutations of P238D and Q295V. In some embodiments, the Fc region comprises a mutation or mutations of P238D and Y296E. In some embodiments, the Fc region comprises a mutation or mutations of P238D and Y296H. In some embodiments, the Fc region comprises a mutation or mutations of P238D and S298M. In some embodiments, the Fc region comprises a mutation or mutations of P238D and S324N. In some embodiments, the Fc region comprises a mutation or mutations of P238D and S324H. In some embodiments, the Fc region comprises a mutation or mutations of P238D and A330H. In some embodiments, the Fc region comprises a mutation or mutations of P238D and A330Y. In some embodiments, the Fc region comprises a mutation or mutations of P238D and F241M, H268P, Y296E and S324H. In some embodiments, the Fc region comprises a mutation or mutations of G237Q, P238D, F241M, Y296E and A330H. In some embodiments, the Fc region comprises a mutation or mutations of L235F, G237Q, P238D, F241M and Y296E.

In some embodiments, the Fc region comprises a mutation or mutations of P238D, P271G and E233D. In some embodiments, the Fc region comprises a mutation or mutations of P238D, P271G and L234R. In some embodiments, the Fc region comprises a mutation or mutations of P238D, P271G and G237D. In some embodiments, the Fc region comprises a mutation or mutations of P238D, P271G and G237K. In some embodiments, the Fc region comprises a mutation or mutations of P238D, P271G and V264I. In some embodiments, the Fc region comprises a mutation or mutations of P238D, P271G and S267A. In some embodiments, the Fc region comprises a mutation or mutations of P238D, P271G and H268E. In some embodiments, the Fc region comprises a mutation or mutations of P238D, P271G and H268P. In some embodiments, the Fc region comprises a mutation or mutations of P238D, P271G and Y296D. In some embodiments, the Fc region comprises a mutation or mutations of P238D, P271G and Y296E. In some embodiments, the Fc region comprises a mutation or mutations of P238D, P271G, E233D, L234K, V264I, S267A and H268E. In some embodiments, the Fc region comprises a mutation or mutations of P238D, P271G, E233D, L234R, V264I, S267A and H268E. In some embodiments, the Fc region comprises a mutation or mutations of P238D, P271G, E233D, G237K, V264I, S267A and H268E. In some embodiments, the Fc region comprises a mutation or mutations of P238D, P271G, E233D, V264I, D265N, S267A and H268E. In some embodiments, the Fc region comprises a mutation or mutations of P238D, P271G, E233D, V264I, S267R and H268E. In some embodiments, the Fc region comprises a mutation or mutations of P238D, P271G, E233D, G237D, V264I, S267Y, H268E, Y296D, A330R and P396M. In some embodiments, the Fc region comprises a mutation or mutations of P238D, P271G, E233D, G237D, V264I, S267A, H268E, Y296D/Y296A, A330R and P396M. In some embodiments, the Fc region comprises a mutation or mutations of P238D, P271G, E233D, V264I, S267R, H268E and Y296E. In some embodiments, the Fc region comprises a mutation or mutations of P238D, P271G, E233D, V264I, S267R and H268P. In some embodiments, the Fc region comprises a mutation or mutations of P238D, P271G, E233D, F241M, V264I, S267R and H268E. In some embodiments, the Fc region comprises a mutation or mutations of P238D, P271G, E233D, V264I, S267R, H268P and Y296E. In some embodiments, the Fc region comprises a mutation or mutations of P238D, P271G, E233D, G237Q, V264I, S267R, H268P and Y296E. In some embodiments, the Fc region comprises a mutation or mutations of E233D, G237D, P238D, H268D, P271G, and A330R.

The mutations and positions of the Fc region, which can also be referred to as the Fc domain, are according to EU numbering.

As used herein, a Fc region/domain comprising a mutation at a specific position is as compared to the wild-type Fc according the numbering system (EU numbering) as referenced herein.

In some embodiments, the Fc domain is linked to the inhibitory receptor effector domain. In some embodiments, the Fc domain is linked to a C-terminus of the inhibitory receptor effector domain. In some embodiments, when the inhibitory receptor effector domain is an antibody, the Fc domain is linked to the C-terminus of the heavy chain of the antibody that forms the inhibitor receptor effector domain. In some embodiments, the N-terminus of the Fc domain is linked to the C-terminus of the inhibitory receptor effector domain. In some embodiments, the Fc domain is directly linked, such as without a linker sequence, to the inhibitory receptor effector domain. In some embodiments, the Fc domain is linked to the inhibitory receptor effector domain through a linker, such as a peptide linker. In some embodiments, the linker is as provided for herein.

In some embodiments, the Fc domain is also linked to the FcγRII binding effector domain. In some embodiments, the C-terminus of the Fc domain is linked to the N-terminus of the FcγRII binding effector domain. In some embodiments, the N-terminus of the Fc domain is linked to a C-terminus of the inhibitory receptor effector domain and the C-terminus of the Fc domain is linked to the N-terminus of the FcγRII binding effector domain. In some embodiments, the Fc domain is linked to the FcγRII binding effector domain directly, such as without a peptide linker. In some embodiments, the Fc domain is linked to the FcγRII binding effector domain through a peptide linker.

Examples of peptide linkers that can be used are known in the art and non-limiting examples are provide for herein.

As used herein, the term “FcγRII binding effector domain” refers to a polypeptide, such as an antibody, that binds to FcγRII receptor. Examples of such receptors include the FcγRIIa or FcγRIIb receptor. In some embodiments, the FcγRII binding effector domain is an antibody. In some embodiments, the FcγRII binding effector domain is a scFv antibody. In some embodiments, the N-terminus of the FcγRII binding effector domain is bound to the C-terminus of the Fc domain. In some embodiments, the FcγRII binding effector domain selectively binds to the FcγRIIb receptor. In some embodiments, the FcγRII binding effector domain selectively binds to the FcγRIIb receptor over the FcγRIIa receptor.

In some embodiments, the polypeptide does not comprise a Fc domain. Thus, in some embodiments, a compound is provided that comprise one or more inhibitor receptor effector domains linked to a FcγRII binding effector domain. In some embodiments, the C-terminus of the inhibitory receptor effector domain is linked to the N-terminus of the FcγRII binding effector domain. In some embodiments, the N-terminus of the inhibitory receptor effector domain is linked to the C-terminus of the FcγRII binding effector domain. In some embodiments, the different domains are linked together through a peptide linker. Non-limiting examples of such linkers are provided for herein.

Antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, Md., Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide. Effector, as that term is used herein, refers to an entity, e.g., a cell or molecule, e.g., a soluble or cell surface molecule, which mediates an immune response. In some embodiments, the effector is an antibody. In some embodiments, the effectors binding domains as provided for herein, refers to a polypeptide (e.g.) that has sufficient binding specificity that it can bind the effector with sufficient specificity that it can serve as an effector binding/modulating molecule. In some embodiments, it binds to effector with at least 10, 20, 30, 40, 50, 60, 70, 80, 90, or 95% of the affinity of the naturally occurring counter-ligand. In some embodiments, it has at least 60, 70, 80, 90, 95, 99, or 100% sequence identity, or substantial sequence identity, with a naturally occurring counter-ligand for the effector.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

In some embodiments, the inhibitory effector binding domain can be referred to as an inhibitory immune checkpoint molecule. This can refer to a polypeptide that can bind to the checkpoint molecule and agonize its cognate inhibitory activity. For example, the antibody can be an anti-PD-1 antibody that binds to PD-1 and agonizes PD-1's activity. In some embodiments, the antibody inhibits the inhibitory checkpoint activity, such that it antagonizes the inhibitory activity. For example, the antibody can be an anti-PD-1 antibody that binds to PD-1 and antagonizes PD-1's activity. The same can be done if the target is any of the inhibitory receptors, such as those provided for herein. In some embodiments, the inhibitory checkpoint receptor is LAG-3. In some embodiments, the inhibitory checkpoint receptor is as provided for herein. These are non-limiting examples and other inhibitory checkpoint receptors can be agonized or antagonized as provided for herein.

The domains can have similarity to those as provided for herein or those that are incorporated by reference. Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, such as those encoding for the domains, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence. For example, a Fc variant can have the sequence of a Fc domain but comprise a mutation that affects its binding to the FcγRIIa or FcγRIIb receptor. In some embodiments, the Fc variant selectively binds to the FcγRIIb receptor. In some embodiments, the Fc variant selectively binds to the FcγRIIb receptor over the FcγRIIa receptor.

Calculations of homology or sequence identity between sequences (the terms are used interchangeably herein) can be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and) (BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the)(BLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g.,)(BLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The present disclosure provides, for example, effector domains that can act as PD-1 agonists. Without being bound to any particular theory, agonism of PD-1 inhibits T cell activation/signaling and can be accomplished by different mechanisms. For example cross-linking can lead to agonism, bead-bound, functional PD-1 agonists have been described (Akkaya. Ph.D. Thesis: Modulation of the PD-1 pathway by inhibitory antibody superagonists. Christ Church College, Oxford, U K, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems) which is hereby incorporated by reference, which acts as an agonist when soluble (Said et al., 2010, Nat Med) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1).

Antibodies against PD-1 and uses therefor, which is incorporated by reference), or can be a bi-specific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1.

PD-1 agonism can be measured by any method, such as the methods described in the examples. For example, cells can be constructed that express, including stably express, constructs that include a human PD-1 polypeptide fused to a b-galactosidase “Enzyme donor” and 2) a SHP-2 polypeptide fused to a b-galactosidase “Enzyme acceptor.” Without being bound by any theory, when PD-1 is engaged, SHP-2 is recruited to PD-1. The enzyme acceptor and enzyme donor form a fully active b-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNγ, IL-17) or other markers, such as CD69 activation marker. Thus, PD-1 agonism can be measured by either cytokine production or cell proliferation. Other methods can also be used to measure PD-1 agonism.

PD-1 is an Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (eg tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein as an effector molecule binding/modulating moiety, which can accomplish localized tissue-specific immunomodulation when paired with a targeting moiety.

Other examples of PD-1 antibodies that can be used include, but are not limited to, those described in JP6278224B2, JP2018518540A, CN1753912B, JP6174321B2, US20200190187A1, U.S. Ser. No. 10/676,516B2, WO2011082400A2, JP2017537090A, JP2012501670A, US2019/0270818, or CC-9000, each of which is hereby incorporated by reference in its entirety.

As provided for herein, in some embodiments, the inhibitory receptor effector domain binds to LAG-3. In some embodiments, the antibody is as described in Angin et al., J Immunol Feb. 15, 2020, 204 (4) 810-818; KR20180004094A, EP3798234A1, and KR20180021833A, each of which is hereby incorporated by reference in its entirety.

The compounds provided for herein can be used to treat auto-immune diseases. Thus, in some embodiments, embodiments are provided for methods of treating an autoimmune disease or disorder in a subject. In some embodiments, the methods comprise administering to the subject a compound as provided for herein. In some embodiments, the subject has or is at risk of having an autoimmune disorder. In some embodiments, the autoimmune disorder is Type 1 Diabetes, Multiple Sclerosis, Cardiomyositis, vitiligo, alopecia, inflammatory bowel disease (IBD, e.g. Crohn's disease or ulcerative colitis), Sjogren's syndrome, focal segmented glomerular sclerosis (FSGS), scleroderma/systemic sclerosis (SSc) or rheumatoid arthritis. In some embodiments, the treatment minimizes rejection of, minimizes immune effector cell mediated damage to, prolongs the survival of subject tissue undergoing, or a risk for, autoimmune attack, such as from a transplant.

Other examples of autoimmune disorders and diseases that can be treated with the compounds described herein include, but are not limited to, myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (CKD), autoimmune hepatitis, primary biliary cirrhosis, primary sclerosing cholangitis, antisynthetase syndrome, alopecia areata, autoimmune angioedema, autoimmune progesterone dermatitis, overlap connective tissues disease syndromes, polymyalgia rheumatic, autoimmune urticaria, bullous pemphigoid, cicatricial pemphigoid, dermatitis herpetiformis, discoid lupus erythematosus, epidermolysis bullosa acquisita, erythema nodosum, anti-neutrophil cytoplasmic antibody associated vasculitis, Henoch-Schonlein purpura, Cogan's syndrome, Buerger's disease, Susan's disease, immune complex vasculitis, primary angiitis of the CNS, gestational pemphigoid, hidradenitis suppurativa, lichen planus, lichen sclerosus, linear iga disease (lad), morphea, pemphigus vulgaris, pityriasis lichenoides et varioliformis acuta, mucha-habermann disease, psoriasis, systemic scleroderma, vitiligo, Addison's disease, autoimmune polyendocrine syndrome (APS) type 1, autoimmune polyendocrine syndrome (APS) type 2, juvenile idiopathic arthritis, juvenile dermatomyositis, autoimmune brain disease, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjögren's syndrome, autoimmune enteropathy, Coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, lyme disease (chronic), mixed connective tissue disease (MCTD), palindromic rheumatism, Parry Romberg syndrome, Parsonage-Turner syndrome, psoriatic arthritis, IBD-associated arthritis, reactive arthritis, relapsing polychondritis, retroperitoneal fibrosis, rheumatic fever, rheumatoid arthritis, autoimmune complications of immune checkpoint inhibitors (IRAEs), sarcoidosis, neurosarcoidosis, Schnitzler syndrome, systemic lupus erythematosus (SLE), undifferentiated connective tissue disease (UCTD), dermatomyositis, IgG4 related disease, fibromyalgia, antiphospholipid syndrome, inclusion body myositis, myositis, myasthenia gravis, neuromyotonia, paraneoplastic cerebellar degeneration, polymyositis, acute disseminated encephalomyelitis (ADEM), adult onset Still's disease, acute motor axonal neuropathy, anti-N-Methyl-D-Aspartate (anti-NMDA) receptor encephalitis, warm antibody hemolytic anemia (wAIHA), immune thrombocytopenia, immune thrombotic thrombocytopenia, thrombotic thrombocytopenia, pernicious anemia, aplastic anemia, Evan's syndrome, autoimmune neutropenia, acquired von Willibrand syndrome, recurring fetal loss, Rh mismatch, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, primary biliary sclerosis, glomerulonephritis, glomerular basement membrane disease, multiple sclerosis, Oshtoran syndrome, pediatric autoimmune neuropsychiatric disorder associated with Streptococcus (PANDAS), progressive inflammatory neuropathy, cutaneous lupus erythematosus, restless leg syndrome, pemphigus foliaceus including fogo selvage, transplantation, antibody-mediated rejection, alloantibody hypersensitization, xenoantibody mediated rejection, solid organ rejection, graft vs host disease acute and chronic, stiff person syndrome, Sydenham chorea, transverse myelitis, autoimmune retinopathy, autoimmune uveitis, uveitis, Cogan syndrome, Graves ophthalmopathy, amyotrophic lateral sclerosis (ALS), Parkinson's disease, autoimmune encephalitis, CNS vasculitis, chronic idiopathic demyelinating polyneuropathy (CIDP), keratitis, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, rheumatic heart disease, chronic rhinosinusitis with nasal polyps, allergic bronchoplmonary mycosis, hypersensitivity pneumonitis, rheumatoid arthritis-associated interstitial lung disease (RA-ILD), nonspecific interstitial pneumonia, allergic asthma, infectious disease/vaccination, antibody dependent enhancement (as with dengue virus infection), chronic meningitis, anti-myelin oligodendrocyte glycoprotein (MOG) disease, activated-DLBCL, anti-drug antibody, anti-gene therapy vector antibody (anti-AAV antibody), antibody to therapeutic biologic agents (cytokines, monoclonal antibodies, enzymes, coagulation factors), autoimmune inner ear disease (AIED), Meniere's disease, Behcet's disease, eosinophilic granulomatosis with polyangiitis (EGPA), giant cell arteritis, polyglandular autoimmune endocrine syndromes, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, and the like.

Other examples of potential autoimmune disorders and diseases, as well as autoimmune comorbidities that can be treated with the compounds described herein include, but are not limited to, chronic fatigue syndrome, complex regional pain syndrome, eosinophilic esophagitis, gastritis, interstitial lung disease, POEMS syndrome, Raynaud's phenomenon, primary immunodeficiency, pyoderma gangrenosum, agammaglobulinemia, anyloidosis, anyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Dego' deiase, eczema, eosinophilic gastroenteritis, eosinophilic pneumonia, erythroblastosis fetalsis, fibrodysplasia ossificans progressive, gastrointestinal pemphigoid, hypogammaglobulinemia, idiopathic giant-cell myocarditis, idiopathic pulmonary fibrosis, IgA nephropathy, immunoregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, and the like.

In some embodiments, the autoimmune disorder does not comprise pemphigus vulgaris, pemphigus. In some embodiments, the autoimmune disorder does not comprise pemphigus foliaceus. In some embodiments, the autoimmune disorder does not comprise bullous pemphigoid. In some embodiments, the autoimmune disorder does not comprise Goodpasture's Disease. In some embodiments, the autoimmune disorder does not comprise psoriasis. In some embodiments, the autoimmune disorder does not comprise a skin disorder. In some embodiments, the disorder does not comprise a neoplastic disorder, e.g., cancer.

In some embodiments, the condition to be treated is a neoplastic disorder, such as a cancer. In contrast, to the molecule that is used to treat an autoimmune disorder the molecule is used to antagonize the inhibitor receptor to which the inhibitory receptor effector domain binds to. Additionally, the Fc domain comprises mutations that are not inhibitory, such that they can be used to extend the half-life of the molecule. In some embodiments, the FcγRII binding effector domain binds preferentially to the FcγRIIb binding effector domain.

In some embodiments, the cancer is a solid or liquid tumor. In some embodiments, the liquid or solid tumor include, but are not limited to, hematopoietic cancer, lymphoid cancer, skin cancer, head and neck cancer, genitourinary cancer, blood cancer, lung cancer, breast cancer, brain cancer, esophageal cancer, colorectal cancer, pancreatic cancer, and any combination thereof.

In some embodiments, the polypeptides provided for herein are used in a method of modulating two types of cells, the method comprising contacting the two types of cells with the polypeptide, or a pharmaceutical composition thereof. In some embodiments, one cell is a T-cell, NK Cell, Dendritic cell, and the like, and the second cell is a B-Cell, an antigen presenting cell (APC), or a myeloid cell.

In some embodiments, the polypeptides provided for herein are used in a method of inhibiting an activated immune cell (e.g. T-cell) and the activity of a B-Cell, an antigen presenting cell (APC), or a myeloid cell, the method comprising contacting the activated immune cell and the B Cell or antigen presenting cell with the polypeptide, or a pharmaceutical composition thereof.

In some embodiments, the polypeptides provided for herein are used in a method of activating or enhancing an activated immune cell (e.g. T-cell) and the activity of B-Cell, an antigen presenting cell (APC), or a myeloid cell, the method comprising contacting the activated immune cell and the B Cell or antigen presenting cell with the polypeptide, or a pharmaceutical composition thereof.

The contacting can occur, for example, by administration of the polypeptides provided for herein to a subject.

As provided for herein the domains can be linked to together with a linker domain or region. Any linker region described herein can be used as a linker. Linkers can be for example, glycine/serine linkers. In some embodiments, the linker can comprise one or more repeats of GGGGS (SEQ ID NO: 1). In some embodiments, the linker comprises 1, 2, 3, 4, or 5 repeats. In some embodiments, the linker comprises GGGGSGGGGS (SEQ ID NO: 2). In some embodiments, the linker comprises GGGGSGGGGSGGGGS (SEQ ID NO: 3). In some embodiments, the linker comprises: GGGGS (SEQ ID NO: 1), (GGGGS)₃ (SEQ ID NO: 2), (GGGGS)ₙ (n=1, 2, 3, 4) (SEQ ID NO: 1-4), (Gly)₈ (SEQ ID NO: 5), (Gly)₆ (SEQ ID NO: 6), (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12). These linkers can be used in any of the compounds or compositions provided herein. These peptide linkers are non-limiting examples and other peptide linkers can also be used.

In some embodiments, the polypeptide that is the compound comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of the therapeutic compound. The Fc backbone can be the Fc region as provided for herein and have a mutation as provided for herein. Thus, as provided for herein, the Fc region can selectively bind to FcγRIIb over FcγRIIa.

Thus, in some embodiments, the antibody comprised of F(ab′)2 on an IgG1 Fc backbone can be an anti-PD-1 antibody, an anti-LAG-3, an anti-CTLA4 antibody (or any other antibody that binds to an inhibitory receptor) on an IgG1 Fc. In some embodiments, the scFV segments fused to the C-terminus could be the FcγRII binding effector domain. In some embodiments, the polypeptide comprises two antibodies linked separately to two separate FcγRII binding effector domains. In some embodiments, the F(ab′)2 bind to PD-1 or LAG-3. In some embodiments, one antibody binds to PD-1 and the other binds to LAG-3.

In some embodiments, the FcγRII binding effector domain as provided for herein, for any of the polypeptides provided for herein can be selective for FcγRIIb over the FcγRIIa-R131 isoform or the FcγRIIa-H131 isoform. Without being bound to any particular theory, these FcγRIIb binding effector domain can be used to help down regulate or inhibit an immune response.

In some embodiments, the FcγRII binding effector domain as provided for herein, for any of the polypeptides provided for herein can be selective for FcγRIIa-R131 isoform or the FcγRIIa-H131 isoform over FcγRIIb.

Non-limiting exemplary configurations of therapeutic compounds comprise the following (e.g., in N to C terminal order):

R1-Linker Region A-R2

R3-Linker Region B-R4,
wherein,
   R1, R2, R3, and R4, each independently comprises an effector binding/modulating moiety, e.g., anti-PD1, anti-LAG3, anti-CTLA4, anti-FcγRIIb; or is absent;
   Linker Region A and Linker B comprise moieties that can associate with one another, e.g., Linker A and Linker B, each comprises an Fc moiety provided that an effector binding/modulating moiety and a specific targeting moiety are present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa. In some embodiments, Linker Region A and Linker Region B are both absent.

In some embodiments:
R1 comprises an effector binding/modulating moiety, e.g., anti-PD-1, anti-LAG3, anti-CTLA4, or anti-FcγRIIb, or is absent;
R2 comprises an effector binding/modulating moiety, e.g., anti-PD-1, anti-LAG3, anti-CTLA4, or anti-FcγRIIb;
R3 comprises an effector binding/modulating moiety, e.g., anti-PD-1, anti-LAG3, anti-CTLA4, or anti-FcγRIIb, or is absent;
R4 comprises an effector binding/modulating moiety, e.g., anti-PD-1, anti-LAG3, anti-CTLA4, or anti-FcγRIIb; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-PD-1 antibody;
R2 comprises an anti-LAG3 antibody;
R3 comprises an anti-PD-1 antibody;
R4 comprises an anti-LAG3 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-LAG3 antibody;
R2 comprises an anti-PD-1 antibody;
R3 comprises an anti-LAG3 antibody;
R4 comprises an anti-PD-1 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-LAG3 antibody;
R2 comprises a FcγRIIb-binding moiety;
R3 comprises an anti-LAG3 antibody;
R4 comprises a FcγRIIb-binding moiety; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises a FcγRIIb-binding moiety;
R2 comprises an anti-LAG3 antibody;
R3 comprises a FcγRIIb-binding moiety;
R4 comprises an anti-LAG3 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-PD-1 antibody;
R2 comprises a FcγRIIb-binding moiety;
R3 comprises an anti-PD-1 antibody;
R4 comprises a FcγRIIb-binding moiety; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises a FcγRIIb-binding moiety;
R2 comprises an anti-PD-1 antibody;
R3 comprises a FcγRIIb-binding moiety;
R4 comprises an anti-PD-1 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-PD-1 antibody;
R2 is absent;
R3 comprises an anti-LAG3 antibody;
R4 is absent; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 is absent;
R2 comprises an anti-PD-1 antibody;
R3 is absent;
R4 comprises an anti-LAG3 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-LAG3 antibody;
R2 is absent;
R3 comprises an anti-PD-1 antibody;
R4 is absent; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 is absent;
R2 comprises an anti-LAG3 antibody;
R3 is absent;
R4 comprises an anti-PD-1 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-PD-1 antibody;
R2 comprises a FcγRIIb-binding moiety;
R3 comprises an anti-LAG3 antibody;
R4 comprises a FcγRIIb-binding moiety; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises a FcγRIIb-binding moiety;
R2 comprises an anti-PD-1 antibody;
R3 comprises a FcγRIIb-binding moiety;
R4 comprises an anti-LAG3 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an anti-LAG3 antibody;
R2 comprises a FcγRIIb-binding moiety;
R3 comprises an anti-PD-1 antibody;
R4 comprises a FcγRIIb-binding moiety; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises a FcγRIIb-binding moiety;
R2 comprises an anti-LAG3 antibody;
R3 comprises a FcγRIIb-binding moiety;
R4 comprises an anti-PD-1 antibody; and
Linker Region A and Linker B comprise moieties that can associate with one another, e.g., Linker A and Linker B, each comprises an Fc moiety, provided that one of R1 or R3 is present and one of R2 or R4 is present. Furthermore, Linker A and Linker B, each comprise an Fc moiety that is selective for FcγRIIb. In some embodiments, the Fc moiety that is selective for FcγRIIb has increased selectivity for FcγRIIb. In some embodiments, the Fc moiety that is selective for FcγRIIb has increased affinity for FcγRIIb. In some embodiments, the Fc moiety that is selective for FcγRIIb has increased selectivity and affinity for FcγRIIb. In some embodiments, the Fc moiety that is selective for FcγRIIb has increased selectivity for FcγRIIb over FcγRIIa. In some embodiments, the Fc moiety that is selective for FcγRIIb has increased affinity for FcγRIIb over FcγRIIa. In some embodiments, the Fc moiety that is selective for FcγRIIb has increased selectivity and affinity for FcγRIIb over FcγRIIa.

In some embodiments:
R1 comprises an effector binding/modulating moiety, e.g., anti-PD-1, anti-LAG3, anti-CTLA4, or anti-FcγRIIb, or is absent;
R2 comprises an effector binding/modulating moiety, e.g., anti-PD-1, anti-LAG3, anti-CTLA4, or anti-FcγRIIb;
R3 comprises an effector binding/modulating moiety, e.g., anti-PD-1, anti-LAG3, anti-CTLA4, or anti-FcγRIIb, or is absent;
R4 comprises an effector binding/modulating moiety, e.g., anti-PD-1, anti-LAG3, anti-CTLA4, or anti-FcγRIIb; and
Linker Region A and Linker B are both absent.

In some embodiments:
R1 comprises an anti-PD-1 antibody;
R2 comprises an anti-LAG3 antibody;
R3 comprises an anti-PD-1 antibody;
R4 comprises an anti-LAG3 antibody; and
Linker Region A and Linker B are both absent.

In some embodiments:
R1 comprises an anti-LAG3 antibody;
R2 comprises an anti-PD-1 antibody;
R3 comprises an anti-LAG3 antibody;
R4 comprises an anti-PD-1 antibody; and
Linker Region A and Linker B are both absent.

In some embodiments:
R1 comprises an anti-LAG3 antibody;
R2 comprises a FcγRIIb-binding moiety;
R3 comprises an anti-LAG3 antibody;
R4 comprises a FcγRIIb-binding moiety; and
Linker Region A and Linker B are both absent.

In some embodiments:
R1 comprises a FcγRIIb-binding moiety;
R2 comprises an anti-LAG3 antibody;
R3 comprises a FcγRIIb-binding moiety;
R4 comprises an anti-LAG3 antibody; and
Linker Region A and Linker B are both absent.

In some embodiments:
R1 comprises an anti-PD-1 antibody;
R2 comprises a FcγRIIb-binding moiety;
R3 comprises an anti-PD-1 antibody;
R4 comprises a FcγRIIb-binding moiety; and
Linker Region A and Linker B are both absent.

In some embodiments:
R1 comprises a FcγRIIb-binding moiety;
R2 comprises an anti-PD-1 antibody;
R3 comprises a FcγRIIb-binding moiety;
R4 comprises an anti-PD-1 antibody; and
Linker Region A and Linker B are both absent.

In some embodiments:
R1 comprises an anti-PD-1 antibody;
R2 is absent;
R3 comprises an anti-LAG3 antibody;
R4 is absent; and
Linker Region A and Linker B are both absent.

In some embodiments:
R1 is absent;
R2 comprises an anti-PD-1 antibody;
R3 is absent;
R4 comprises an anti-LAG3 antibody; and
Linker Region A and Linker B are both absent.

In some embodiments:
R1 comprises an anti-LAG3 antibody;
R2 is absent;
R3 comprises an anti-PD-1 antibody;
R4 is absent; and
Linker Region A and Linker B are both absent.

In some embodiments:
R1 is absent;
R2 comprises an anti-LAG3 antibody;
R3 is absent;
R4 comprises an anti-PD-1 antibody; and
Linker Region A and Linker B are both absent.

In some embodiments:
R1 comprises an anti-PD-1 antibody;
R2 comprises a FcγRIIb-binding moiety;
R3 comprises an anti-LAG3 antibody;
R4 comprises a FcγRIIb-binding moiety; and
Linker Region A and Linker B are both absent.

In some embodiments:
R1 comprises a FcγRIIb-binding moiety;
R2 comprises an anti-PD-1 antibody;
R3 comprises a FcγRIIb-binding moiety;
R4 comprises an anti-LAG3 antibody; and
Linker Region A and Linker B are both absent.

In some embodiments:
R1 comprises an anti-LAG3 antibody;
R2 comprises a FcγRIIb-binding moiety;
R3 comprises an anti-PD-1 antibody;
R4 comprises a FcγRIIb-binding moiety; and
Linker Region A and Linker B are both absent.

In some embodiments:
R1 comprises a FcγRIIb-binding moiety;
R2 comprises an anti-LAG3 antibody;
R3 comprises a FcγRIIb-binding moiety;
R4 comprises an anti-PD-1 antibody; and
Linker Region A and Linker B are both absent.

In some embodiments, Linker Region A and Linker B are identical. In some embodiments, Linker Region A and Linker B are different. In some embodiments, Linker Region A and Linker B, each comprise a mutation, mutations, or sets of mutations, such as those provided herein. In some embodiments, Linker Region A and Linker B, each comprise a mutation, mutations, or sets of mutations, such as those provided herein, wherein the mutation, mutations, or sets of mutations, confer increased selectivity for FcγRIIb. In some embodiments, Linker Region A and Linker B, each comprise a mutation, mutations, or sets of mutations, such as those provided herein, wherein the mutation, mutations, or sets of mutations, confer increased affinity for FcγRIIb. In some embodiments, Linker Region A and Linker B, each comprise a mutation, mutations, or sets of mutations, such as those provided herein, wherein the mutation, mutations, or sets of mutations, confer increased selectivity and affinity for FcγRIIb. In some embodiments, Linker Region A and Linker B, each comprise a mutation, mutations, or sets of mutations, such as those provided herein, wherein the mutation, mutations, or sets of mutations, confer increased selectivity for FcγRIIb over FcγRIIa. In some embodiments, Linker Region A and Linker B, each comprise a mutation, mutations, or sets of mutations, such as those provided herein, wherein the mutation, mutations, or sets of mutations, confer increased affinity for FcγRIIb over FcγRIIa. In some embodiments, Linker Region A and Linker B, each comprise a mutation, mutations, or sets of mutations, such as those provided herein, wherein the mutation, mutations, or sets of mutations, confer increased selectivity and affinity for FcγRIIb over FcγRIIa.

In some embodiments, the bispecific antibodies are comprised of four polypeptide chains comprising the following:

Chain 1: nt-VH1-CH1-CH2-CH3-Linker A-scFv[VL2-Linker B-VH2]-ct

Chain 2: nt-VH1-CH1-CH2-CH3-Linker A-scFv[VL2-Linker B-VH2]-ct

Chain 3: nt-VL1-CL-ct

Chain 4: nt-VL1-CL-ct.

In some embodiments, the bispecific antibodies are comprised of four polypeptide chains comprising the following:

Chain 1: nt-VH1-CH1-CH2-CH3-Linker A-scFv[VH2-Linker B-VL2]-ct

Chain 2: nt-VH1-CH1-CH2-CH3-Linker A-scFv[VH2-Linker B-VL2]-ct

Chain 3: nt-VL1-CL-ct

Chain 4: nt-VL1-CL-ct.

In some embodiments, the bispecific antibodies are comprised of four polypeptide chains that do not contain the Fc region, which can be illustrated as having the following formula:

Chain 1: nt-VH1-CH1-Linker A-scFv[VH2-Linker B-VL2]-ct

Chain 2: nt-VH1-CH1-Linker A-scFv[VH2-Linker B-VL2]-ct

Chain 3: nt-VL1-CL-ct

Chain 4: nt-VL1-CL-ct.

In some embodiments, the bispecific antibodies are comprised of four polypeptide chains that do not contain the Fc region, which can be illustrated as having the following formula:

Chain 1: nt-VH1-Linker A-scFv[VH2-Linker B-VL2]-ct

Chain 2: nt-VH1-Linker A-scFv[VH2-Linker B-VL2]-ct

Chain 3: nt-VL1-CL-ct

Chain 4: nt-VL1-CL-ct.

In some embodiments, the bispecific antibodies are comprised of four polypeptide chains that do not contain the Fc region, which can be illustrated as having the following formula:

Chain 1: nt-VH1-CH1-Linker A-scFv[VL2-Linker B-VH2]-ct

Chain 2: nt-VH1-CH1-Linker A-scFv[VL2-Linker B-VH2]-ct

Chain 3: nt-VL1-CL-ct

Chain 4: nt-VL1-CL-ct.

In some embodiments, the bispecific antibodies are comprised of four polypeptide chains that do not contain the Fc region, which can be illustrated as having the following formula:

Chain 1: nt-VH1-Linker A-scFv[VL2-Linker B-VH2]-ct

Chain 2: nt-VH1-Linker A-scFv[VL2-Linker B-VH2]-ct

Chain 3: nt-VL1-CL-ct

Chain 4: nt-VL1-CL-ct.

In some embodiments, chains 1 and 2 are identical to each other, and chains 3 and 4 are identical to each other. In some embodiments, chains 3 and 4 are identical and chains 1 and 2 are different from one another or are different from one another at the N or C terminus or both. In some embodiments, each of the chains have different sequences. In some embodiments, wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g. GGGGSG (SEQ ID NO: 13), GGGGS (SEQ ID NO: 1), GGGGSGGGGSGGGGSGGGGS (SEQ ID NO: 4), GGGGSGGGGSGGGGS (SEQ ID NO: 3) or GGGGSGGGGS (SEQ ID NO: 2)). The sequences of Linker A and Linker B, which are independent of one another can be the same or different and as otherwise described throughout the present application. Thus, in some embodiments, Linker A comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker B comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker A comprises 1, 2, 3, 4, or 5 GGGGS repeats. In some embodiments, Linker B comprises 1, 2, 3, 4, or 5 GGGGS (SEQ ID NO: 1) repeats. For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, Linker B comprises GGGGS (SEQ ID NO: 1), GGGGSGGGGS (SEQ ID NO: 2), GGGGSGGGGSGGGGS (SEQ ID NO: 3), or GGGGSGGGGSGGGGSGGGGS (SEQ ID NO: 4). In some embodiments, the Linker A or Linker B comprises: GGGGS (SEQ ID NO: 1), (GGGGS)₃ (SEQ ID NO: 2), (GGGGS)ₙ (n=1, 2, 3, 4) (SEQ ID NO: 1-4), (Gly)₈ (SEQ ID NO: 5), (Gly)₆ (SEQ ID NO: 6), (EAAAK)₃ (SEQ ID NO: 7), (EAAK)ₙ (n=1-3) (SEQ ID NO: 8-10), A(EAAAK)₄ALEA(EAAAK)₄A (SEQ ID NO: 11), or AEAAAKEAAAKA (SEQ ID NO: 12).

The scFv may also be arranged in the NT-VH2-VL2-CT or NT-VL2-VH2-CT orientation. NT or nt stands for N-terminus and CT or ct stands for C-terminus of the protein. In some embodiments, the CH1, CH2, and CH3 are the domains from the IgG Fc region, and CL stands for Constant Light chain, which can be either kappa or lambda family light chains. The other definitions stand for the way they are normally used in the art. In some embodiments, the CH2 portions when present on the strands are different from one another. In some embodiments, the CH2 portions are the same.

In some embodiments, the compound comprises a light chain and a heavy chain. In some embodiments, the light and heavy chain begin at the N-terminus with the VH domain of a inhibitory receptor effector domain followed by the CH1 domain of a human IgG1, which is fused to a Fc region (e.g. CH2-CH3) of human IgG1. In some embodiments, at the c-terminus of the Fc region is fused to a linker as provided herein, such as but not limited to, GGGGS (SEQ ID NO; 1), GGGGSGGGGS (SEQ ID NO: 2) or GGGGSGGGGSGGGGS (SEQ ID NO: 3). The linker can then be fused to FcγRII binding effector domain. The polypeptides can dimerize because through the heavy chain dimerization, which results in a therapeutic compound having two effector moieties, such as two anti-PD-1 antibodies. However, where the antibodies bind to different molecules, they can form a heterodimer that bind to two different inhibitory receptors, such as, but not limited to those provided for herein, including PD-1 and LAG-3. In this orientation, the targeting moiety is an IgG format, there are two Fab arms that each recognize binding partner of the inhibitory receptor, for example, PD-1 being bound by the anti-PD-1 inhibitory receptor effector domain.

For the sake of clarity, in some embodiments, the VH1 and VL1 can form an antibody binding region that binds to FcγRII (i.e., is the FcγRII binding effector domain) and the scFv is the inhibitory receptor effector domain. In some embodiments, the VH1 and VL1 can form an antibody that is the inhibitory receptor effector domain and the scFv is the antibody that binds to FcγRII (i.e., is the FcγRII binding effector domain).

In addition to the mutations provided for herein, the Fc portion or domain can bear mutations to render the Fc region “effectorless,” that is unable to bind FcRs if that is desired. If the present and the Fc is effectorless, then polypeptide will comprise an FcγRII effector domain that is distinct from the Fc region/domain. The mutations that render Fc regions effectorless are known. In some embodiments, the mutations in the Fc region, which is according to the known numbering system, are selected from the group consisting of: K322A, L235A, L236A, G237A, L235F, L236E, N297, P331S, or any combination thereof. In some embodiments, the Fc mutations comprises a mutation at L235 and/or L236 and/or G237. In some embodiments, the Fc mutations comprise L235A and/or L236A mutations, which can be referred to as LALA. In some embodiments, the Fc mutations comprise L235A, L236A, and G237A mutations, which can be referred to as LALAGA (SEQ ID NO: 14) or AAA.

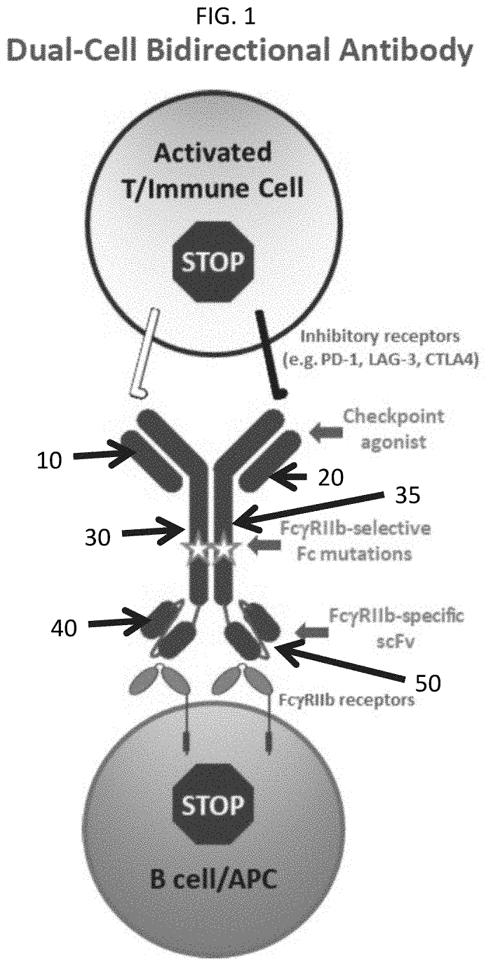

Another non-limiting example of a compound as provided for herein is illustrated in FIG. 1. Referencing FIG. 1, illustrates a dual targeted (bidirectional) antibody that can bind to two different cells at the same time or nearly simultaneously. Referencing FIG. 1 (10) illustrates a inhibitory receptor effector domain as an antibody (e.g. F′Ab2) that binds to an inhibitory receptor, such as PD-1, LAG3, or CTLA4. (20) illustrates another inhibitory receptor effector domain as an antibody that binds to an inhibitory receptor. The inhibitory receptor domains of (10) and (20) can bind to the same inhibitory receptor or different inhibitory receptors. Although illustrated as being a checkpoint agonist, the inhibitory receptor effector domains of (10) and (20) can be checkpoint antagonists as described herein.

Referencing FIGS. 1, (30) and (35) illustrate Fc domains that can comprise FcγRIIb selective mutations. Although illustrated as having FcγRIIb selective mutations the Fc domain can also instead harbor FcγRIIa selective mutations. In such embodiments, if the Fc domain comprises FcγRIIa selective mutations, the inhibitory receptor effector domain can comprise an inhibitory checkpoint antagonist.

Referencing FIG. 1, (40) and (50) illustrate FcγRII binding effector domains, which are shown as a scFv antibody. In some embodiments, (40) and (50) are the same, but they can have different structures, i.e. sequences. Additionally, (40) and (50) are illustrated as being FcγRIIb-specific scFv antibodies. However, (40) and (50) can also be being FcγRIIa-specific scFv antibodies.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approach. In some embodiments, the dimerization interface centers around human IgG CH2-CH3 domains of the Fc domains selective for FcγRIIb, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homo-dimer are different from those at the C-terminus of the homo-dimer. Using this type of bivalency for both an inhibitory T cell receptor at either terminus of the molecule and bivalency for an FcγRIIb receptor can be achieved at either end of the molecule.

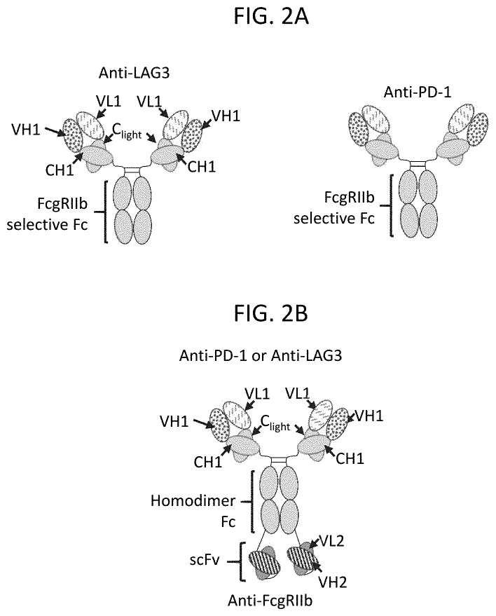

For example, in FIG. 2A, a non-limiting embodiment is illustrated. The N-terminus of the homodimer contains two identical Fab domains comprised of two identical light chains, which are separate polypeptides, interfaced with the N-terminal VH1-CH1 domains of each heavy chain via the VH/VL interaction and Clight interaction with CH1. The native disulfide bond between the Clight and CH1 is present providing a covalent anchor between the light and heavy chains. Further in FIG. 2B, at the C-terminus of the design shown in FIG. 2A are two identical scFv units where by (in this example) the C-terminus of the CH3 domain of the Fc, is followed by a flexible, hydrophilic linker typically comprised of (but not limited to) serine, glycine, alanine, and/or threonine residues, which is followed by the VH2 domain of each scFv unit, which is followed by a glycine/serine rich linker, followed by a VL2 domain. These tandem VH2 and VL2 domains associate to form a single chain fragment variable (scFv) appended at the C-terminus of the Fc. Two such units exist at the C-terminus of this molecule owing to the homodimeric nature centered at the Fc. The domain order of scFvs may be configured to be from N to C terminus either VH-Linker-VL or VL-Linker-VH.

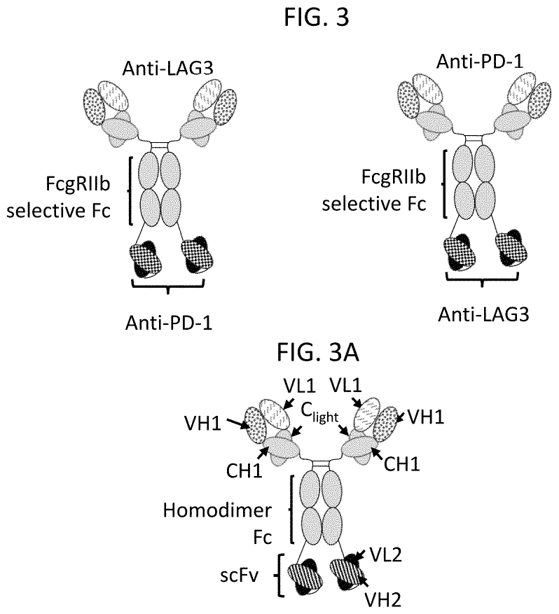

A non-limiting example of a molecule that has different binding regions on the different ends is where, one end is an anti-PD-1 antibody and the other end is an anti-LAG3 antibody. This can be illustrated as shown, for example, in FIG. 3, which illustrates the molecules in different orientations.

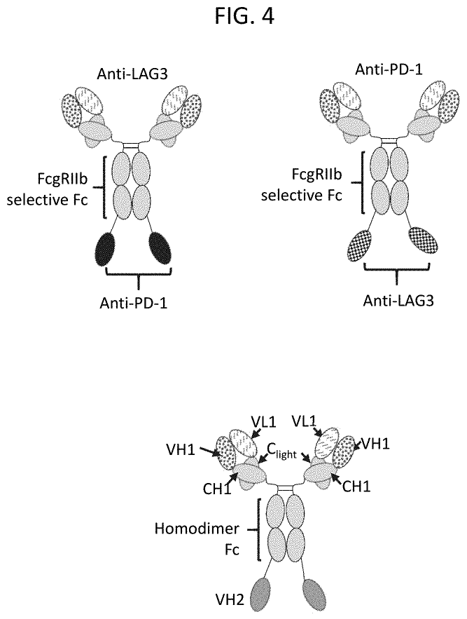

In another example, and as depicted in FIG. 4, the N-terminus of the homodimer contains two identical Fab domains comprised of two identical light chains, which are separate polypeptides, interfaced with the N-terminal VH1-CH1 domains of each heavy chain via the VH/VL interaction and Clight interaction with CH1. The native disulfide bond between the Clight and CH1 is present providing a covalent anchor between the light and heavy chains. At the C-terminus of this design are two identical VH2 units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH2 domain. Two such units exist at the C-terminus of this molecule owing to the homodimeric nature centered at the Fc.

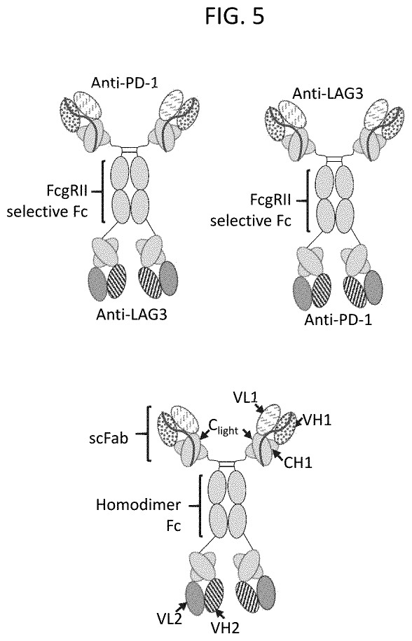

In another non-limiting example, as depicted in FIG. 5, the N-terminus of the homodimer contains two identical single chain Fab (scFab) domains comprised of two identical light chains, which, unlike FIG. 3 and FIG. 4, are physically conjoined with the heavy chain at the N-terminus via a linker between the C-terminus of Clight and the N-terminus of the VH1. The linker may be 36-80 amino acids in length and comprised of serine, glycine, alanine and threonine residues. The physically conjoined N-terminal light chains interface with the N-terminal VH1-CH1 domains of each heavy chain via the VH/VL interaction and Clight interaction with CH1. The native disulfide bond between the Clight and CH1 is present providing additional stability between the light and heavy chains. At the C-terminus of this design are two identical Fab units whereby (in this example) the C-terminus of the CH3 domain of the Fc, is followed by a flexible, hydrophilic linker typically comprised of (but not limited to) serine, glycine, alanine, and/or threonine residues, which is followed by a CH1 domain, followed by a VH2 domain at the C-terminus. The light chain that is designed to pair with the C-terminal CH1/VH2 domains is expressed as a separate polypeptide, unlike the N-terminal light chain which is conjoined to the N-terminal VH1/CH1 domains as described. The C-terminal light chains form an interface at between VH/VL and Clight with CH1. The native disulfide anchors this light chain to the heavy chain. Again, any of the antibody moieties at any of the four attachment/fusion points can be substituted with a non-antibody moiety, e.g., a effector binding/modulating moiety that does not comprise an antibody molecule.

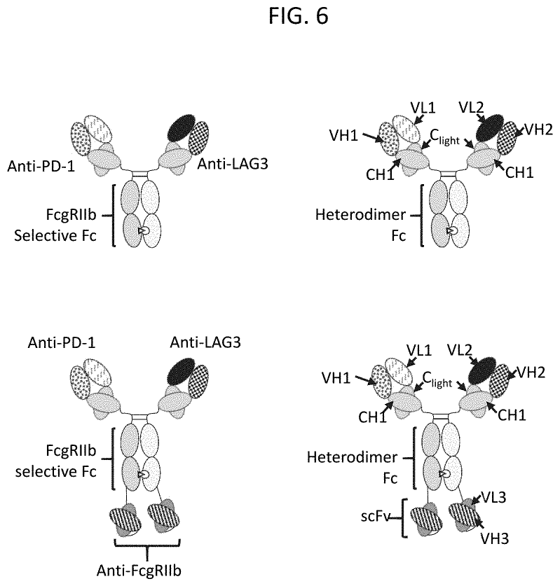

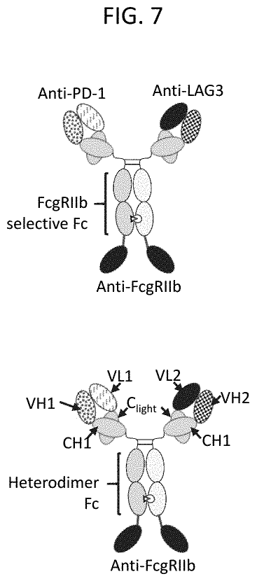

The bispecific antibodies can also be asymmetric as shown in the following non-limiting examples. FIG. 6 and FIG. 7 illustrate an asymmetric/heterodimer approach. In any of these formats, any of the antibody moieties at any of the four attachment/fusion points can be substituted with a non-antibody moiety, e.g., a effector binding/modulating moiety that does not comprise an antibody molecule. In some embodiments, the dimerization interface centers around the human IgG CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes and which could achieve recognition of two different epitopes on the same effector moiety target, or could recognize for examples a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. At the C-terminus of the molecule are two identical scFv units whereby, in this example, the C-terminus of the CH3 domain of the Fc, is followed by a flexible, hydrophilic linker typically comprised of (but not limited to) serine, glycine, alanine, and/or threonine residues, which is followed by the VH2 domain of each scFv unit, which is followed by a glycine/serine rich linker, followed by a VL2 domain. These tandem VH2 and VL2 domains associate to form a single chain fragment variable (scFv) appended at the C-terminus of the Fc. Two such units exist at the C-terminus of this molecule owing to the homodimeric nature centered at the Fc. The domain order of scFvs may be configured to be from N to C terminus either VH-Linker-VL or VL-Linker-VH.

Another example of an asymmetric molecule is depicted in FIG. 7. Referring to FIG. 7, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. At the C-terminus of the molecule are two identical soluble VH germline based VH domains, which are identical on each of the two heavy chains. The heavy chain heterodimerizes via the previously described knobs into holes mutations present at the CH3 interface of the Fc module.

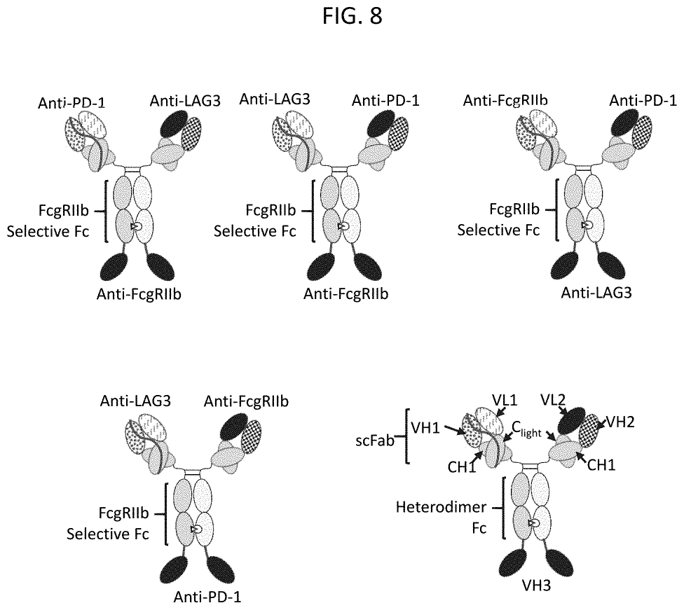

Referring to FIG. 8, the N-terminus of the molecule is comprised of a first light chain paired with a first heavy chain via VH/VL and Clight with CH1 interactions, and a covalent tether comprising the native heavy/light chain disulfide bond. On the opposite side of this heterodimeric molecule at the N-terminus is a second light chain and a second heavy chain which are physically conjoined via a linker between the C-terminus of Clight and the N-terminus of the VH1. The linker may be 36-80 amino acids in length and comprised of serine, glycine, alanine and threonine residues. The physically conjoined N-terminal light chains interface with the N-terminal VH1-CH1 domains of each heavy chain via the VH/VL interaction and Clight interaction with CH1. The native disulfide bond between the Clight and CH1 is present providing additional stability between the light and heavy chains. At the C-terminus of this molecule are two identical soluble VH3 germline family VH domains joined via an N-terminal glycine/serine/alanine/threonine based linker to the C-terminus of the CH3 domain of both heavy chain 1 and heavy chain 2 of the Fc dimer.

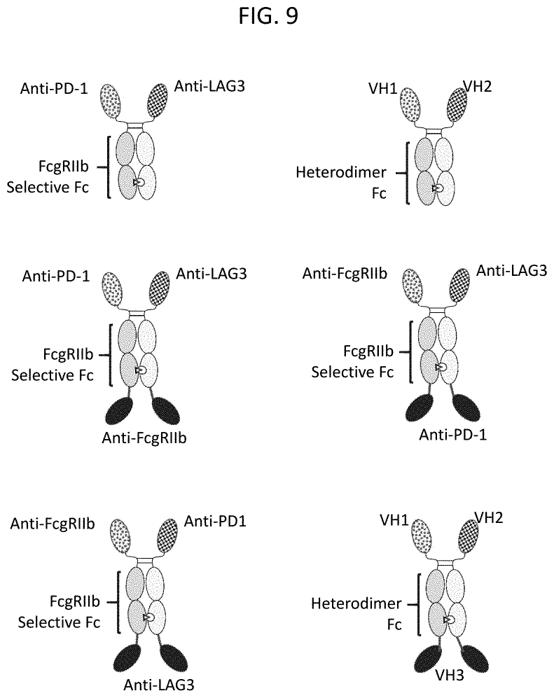

In some embodiments, an asymmetric molecule can be as illustrated as depicted in FIG. 9. For example, the N-terminus of the molecule is comprised of two different VH germlined based soluble VH domains linked to the human IgG1 hinge region via a glycine/serine/alanine/threonine based linker. The VH domain connected to the first heavy chain is different than the VH domain connected to the second heavy chain. At the C-terminus of each heavy chain is an additional soluble VH germline based VH domain, which is identical on each of the two heavy chains. The heavy chain heterodimerizes via the previously described knobs into holes mutations present at the CH3 interface of the Fc module.

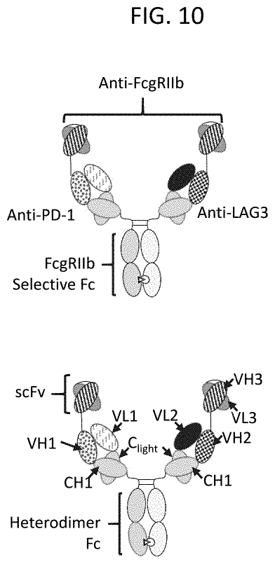

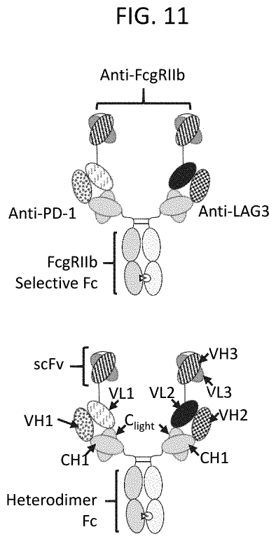

Other embodiments of trispecific molecules are illustrated in FIGS. 10 and 11. As illustrated in FIG. 10, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. Further at the N-terminus of the molecule are two identical scFv units whereby, in this example, the N-terminus of the VH1 or VH2 domain of either Fab moiety, is followed by a flexible, hydrophilic linker typically comprised of (but not limited to) serine, glycine, alanine, and/or threonine residues, which is followed by the VH3 domain of each scFv unit, which is followed by a glycine/serine rich linker, followed by a VL3 domain. These tandem VH3 and VL3 domains associate to form a single chain fragment variable (scFv) domains appended at the N-terminus of the Fab molecule. As illustrated in FIG. 11, the N-terminus of the molecule is comprised of a first light chain paired with a first heavy chain via VH/VL and Clight with CH1 interactions and a covalent tether comprised of the native heavy/light chain disulfide bond. On the opposite side of this heterodimeric molecule at the N-terminus is a second light chain paired with a second heavy chain via VH/VL and Clight with CH1 interactions and a covalent tether comprised of the native heavy/light chain disulfide bond. The physically conjoined N-terminal light chains interface with the N-terminal VH-CH1 domains of each heavy chain via the VH/VL interaction and Clight interaction with CH1. The native disulfide bond between the Clight and CH1 is present providing additional stability between the light and heavy chains. Further at the N-terminus of the molecule are two identical scFv units whereby, in this example, the N-terminus of the VL1 or VL2 domain of either Fab moiety, is followed by a flexible, hydrophilic linker typically comprised of (but not limited to) serine, glycine, alanine, and/or threonine residues, which is followed by the VH3 domain of each scFv unit, which is followed by a glycine/serine rich linker, followed by a VL3 domain. These tandem VH3 and VL3 domains associate to form a single chain fragment variable (scFv) domains appended at the N-terminus of the Fab molecule.

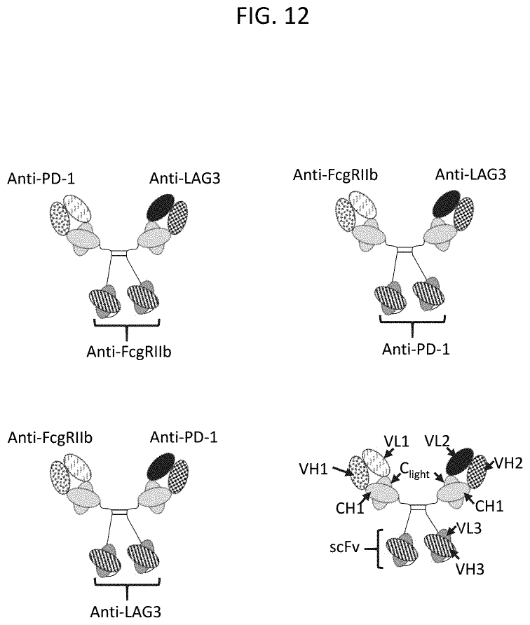

FIG. 12 illustrates another embodiment. FIG. 12 is a F(ab′)2 scFv fusion. This consists of two Fab components having different specificity, joined via two disulfide bonds in the native human IgG hinge region C-terminal of the human IgG CH1 domain. The human IgG CH2 and CH3 domains are absent. At the C-terminus of heavy chains 1 and 2 are two identical scFv fragments linked via a gly/ser/ala/thr rich linker to the C-terminus of the human IgG hinge region. In the configuration shown, the VH is N-terminal in each scFv unit and linked via a 12-amino acid gly/ser rich linker to the N-terminus of a VL domain. An alternative configuration would be N-terminus-VL-Linker-VH-C-terminus. In this design, the construct is trispecific. Again, in this format, any of the antibody moieties at any of the four attachment/fusion points can be substituted with a non-antibody moiety, e.g., a effector binding/modulating moiety that does not comprise an antibody molecule.

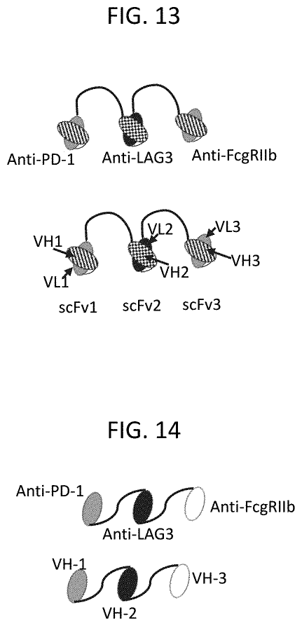

FIG. 13 represents a tandem scFv format consisting of a first N-terminal VL domain linked at its C-terminus to the N-terminus of a first VH domain with a 12-15 amino acid gly/ser rich linker, followed at the first VH C-terminus by a 25-30 amino acid gly/ser/ala/thr based linker to the N-terminus of a second VL domain. The second VL domain is linked at the C-terminus to the N-terminus of a second VH domain by a 12-15 amino acid gly/ser linker, followed at the second VH C-terminus by a 25-30 amino acid gly/ser/ala/thr based linker to the N-terminus of a third VL domain. The third VL domain is linked at the C-terminus to the N-terminus of a third VH domain by a 12-15 amino acid gly/ser linker. Each scFv recognizes a different target antigen such as a co-stimulatory T cell molecule and a FcγRIIb receptor. In this format, any of the antibody moieties can be substituted with a non-antibody moiety, e.g., a effector binding/modulating moiety that does not comprise an antibody molecule.

FIG. 14 illustrates another example. In this example, the molecule is comprised of three VH germline based soluble VH domains. Each of the three domains has different specificity. For example, the first domain from the N-terminus may have specificity for an inhibitory receptor, the second domain from the N-terminus may have specificity for another inhibitory receptor, and the third domain from the N-terminus may have specificity for a tissue antigen and the fourth domain from the N-terminus may have specificity for FcγRIIb. Two glycine, serine, alanine and/or threonine rich linkers exists between domains 1 and 2, and domains 2 and 3. This format may be configured with up to trispecificity, but monovalent in each case, or to have bispecificity with bivalency in each case. The order of domains can be changed. Again, in this format, any of the antibody moieties can be substituted with a non-antibody moiety, e.g., a effector binding/modulating moiety that does not comprise an antibody molecule.

In some embodiments, when the inhibitory receptor effector domain is a checkpoint agonist, the Fc domain comprises mutations that are FcγRIIb selective mutations and the FcγRII binding effector domains is a FcγRIIb-specific scFv antibody.

In some embodiments, when the inhibitory receptor effector domain is a checkpoint antagonist, the Fc domain comprises mutations that are FcγRIIa selective mutations and the FcγRII binding effector domains is a FcγRIIa-specific scFv antibody.

Pharmaceutical Compositions and Kits

In some embodiments, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, local, ophthalmic, topical, spinal or epidermal administration (e.g. by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The carriers can be used in pharmaceutical compositions comprising the therapeutic compounds provided for herein.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

### ENUMERATED EMBODIMENTS

1. A polypeptide comprising:
   i) an inhibitory receptor effector domain;
   ii) an Fc region; and/or a FcγRII binding effector domain,
   wherein the Fc region selectively binds to FcγRIIb; and
   wherein the FcγRII binding effector domain selectively binds to FcγRIIb or FcγRIIa, provided that when:
   i) the Fc region selectively binds to FcγRIIb, the FcγRII binding effector domain selectively binds to FcγRIIb when both the Fc region and the FcγRII binding effector domain present; or
   ii) the Fc region selectively binds to FcγRIIa, the FcγRII binding effector domain selectively binds to FcγRIIa when both the Fc region and the FcγRII binding effector domain present.
2. The polypeptide of embodiment 1, wherein the polypeptide comprises 2 inhibitory receptor effector domains.
3. The polypeptide of embodiment 2, wherein the 2 inhibitory receptor effector domains bind to the same inhibitory receptor.
4. The polypeptide of embodiment 2, wherein the 2 inhibitory receptor effector domains bind to different inhibitory receptors.
5. The polypeptide of any one of the preceding embodiments, wherein the inhibitory receptor effector domain is an antibody.
6. The polypeptide of any one of the preceding embodiments, wherein the inhibitory receptor effector domain is, independently, an antibody in the format of an scFv, a Fab, a Fab′, or a F(ab′)2 antibody.
7. The polypeptide of any one of the preceding embodiments, wherein the inhibitor receptor effector domain binds to a receptor encoded by LAG3, PDCD1, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLECS, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, ILT3, ILT4, ILT5, LIRE, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CDS, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.
8. The polypeptide of any one of the preceding embodiments, wherein the inhibitory receptor effector domain binds to PD-1, LAG-3, or CTLA4.
9. The polypeptide of any one of the preceding embodiments, wherein the inhibitory receptor effector domain is an agonist of the inhibitory receptor to which it binds.
10. The polypeptide of any one of the preceding embodiments, wherein the inhibitory receptor effector domain is an antagonist of the inhibitory receptor to which it binds.
11. The polypeptide of any one of the preceding embodiments, wherein the Fc region selectively binds to FcγRIIb.
12. The polypeptide of any one of the preceding embodiments, wherein the Fc region selectively binds to FcγRIIb and the polypeptide does not comprise a separate FcγRII binding effector domain.
13. The polypeptide of any one of embodiments 11-12, wherein the Fc region that selectively binds to FcγRIIb comprises a mutation, mutations, or a set of mutations, as compared to a wild-type Fc region.
14. The polypeptide of embodiment 13, wherein the mutation, mutations, or set of mutations is selected from P238D; P238D and E233D; P238D and L234W; P238D and L234Y; P238D and G237W; P238D and G237F; P238D and G237A; P238D and G237D; P238D and G237E; P238D and G237L; P238D and G237M; P238D and G237Y; P238D and S239D; P238D and S267V; P238D and S267Q; P238D and S267A; P238D and H268N; P238D and H268D; P238D and H268E; P238D and P271G; P238D and Y296D; P238D and V323I; P238D and V323L; P238D and V323M; P238D and K326L; P238D and K326Q; P238D and K326E; P238D and K326M; P238D and K326D; P238D and K326S; P238D and K326T; P238D and K326A; P238D and K326N; P238D and L328E; P238D and A330K; P238D and A330R; P238D and A330M; S239P; S239P and P230E; S239P and A231D; S239P and P232E; S239P and P238E; S239P, P230E and A231D; S239P, P230E and P232E; S239P, P230E and P238E; S239P, P230E, A231D and P232E; S239P, P230E, A231D and P238E; S239P, P230E, A231D, P232E and P238E; S239P, A231D and P232E; S239P, A231D and P238E; S239P, A231D, P232E and P238E; S239P, P232E and P238E; S267E; S267D; S267E and L328F; G236D and S267E; S239D and S267E; S239D and I332E; K409E; L368K; S364D and K370G; S364Y and K370R; S364D; Y349K; K409D; K392E; D399K; S364E; L368E and K409E; S364E and F405A; Y349K and T394F; S364H and Y349K; P395T, V397S and F405A; T394F; T394S, P395V, P396T, V397E and F405S; V397S and F405A; S364H, D401K and F405A; Y349T, T394F and T411E; L351K, S364H and D401K; Y349T, L351E and T411E; S364H; Y349T; S364H and D401K; Y349T and T411E; S364H and T394F; Y349T and F405A; S364H and F405A; Y349T and T394F; F405A; S364E and T394F; Y349K and F405A; V397T and F405S; S364E and F405S; Y349K and T394Y; S364E, T411E and F405A; Y349K, T394F and D401K; S364E and T411E; Y349K and D401K; L351E and S364D; Y349K and L351K; L351E and S364E; Y349C and S364E; Y349K and S354C; S364H, F405A and T411E; Y349T, T394F and D401K; S364D and T394F; L235Y; L235R; G236D; L328F; L235Y, G236D, S267D and L328F; L235Y, G236D and S267D; L235Y, G236D and S267E; L235Y and G236D; L235Y, S267D and L328F; L235Y, S267E and L328F; L235Y and L328F; L235R, G236D, S267D and L328F; L235R, G236D and S267D; L235R, G236D and S267E; L235R and G236D; L235R, S267D and L328F; L235R, S267E and L328F; L235R and L328F; G236D, S267E and L328F; G236D, S267D and L328F; G236D and L328F; S267D and L328F; G236N and S267E; G236N; L234Y, L235Y, G236W, H268D and S298A; L234Y, L235Y, G236W, H268D, D270E and S298A; L234Y, L235Q, G236W, S239M, H268D, D270E and S298A; L234Y, L235Y, G236W, H268D, S298A and A327D; L234Y, L235Y, G236W, S239M, H268D, S298A and A327D; L234Y, L235Y, G236W, S239M, H268D, S298A, A327D, L328W and K334L; second IgG1 CH2 Domain; K326D, A330M and K334E; D270E, K326D, A330M and K334E; D270E, K326D, A330K and K334E; L234E, L235Y, G236W, S239M, H268D, S298A and A327D; L234S, L235Y, G236W, S239M, H268D, S298A and A327D; L235Q, G236W, S239M, H268D, D270E and S298A; L235Y, G236W, S239M, H268D, S298A and A327D; L234S, L235Q, G236W, S239M, H268D, D270E and S298A; L234F, L235Q, G236W, S239M, H268D, D270E and S298A; L234E, L235Q, G236W, S239M, H268D, D270E and S298A; L234F, L235Y, G236W, S239M, H268D, S298A and A327D; L234V, L235Q, G236W, S239M, H268D, D270E and S298A; L234D, L235Q, G236W, S239M, H268D, D270E and S298A; L234Q, L235Q, G236W, S239M, H268D, D270E and S298A; L234I, L235Q, G236W, S239M, H268D, D270E and S298A; L234M, L235Q, G236W, S239M, H268D, D270E and S298A; L234T, L235Q, G236W, S239M, H268D, D270E and S298A; L234A, L235Q, G236W, S239M, H268D, D270E and S298A; L234G, L235Q, G236W, S239M, H268D, D270E and S298A; L234H, L235Q, G236W, S239M, H268D, D270E and S298A; L234V, L235Y, G236W, S239M, H268D, S298A and A327D; L234D, L235Y, G236W, S239M, H268D, S298A and A327D; L234Q, L235Y, G236W, S239M, H268D, S298A and A327D; L234I, L235Y, G236W, S239M, H268D, S298A and A327D; L234M, L235Y, G236W, S239M, H268D, S298A and A327D; L234T, L235Y, G236W, S239M, H268D, S298A and A327D; L234A, L235Y, G236W, S239M, H268D, S298A and A327D; L234G, L235Y, G236W, S239M, H268D, S298A and A327D; L234H, L235Y, G236W, S239M, H268D, S298A and A327D; L234F, L235Q, G236W, S239I, H268D, D270E and S298A; L234E, L235Q, G236W, S239I, H268D, D270E and S298A; L234D, L235Q, G236W, S239I, H268D, D270E and S298A; L234V, L235Y, G236W, S239I, H268D, S298A and A327D; L234I and L235Y, G236W, S239I, H268D, S298A, A327D; L235Y, G236W, S239I, H268D, S298A, A327D; L234E, L235Y, G236W, S239I, H268D, S298A and A327D; L234D, L235Y, G236W, S239I, H268D, S298A and A327D; L234F, L235Y, G236W, S239I, H268D, S298A and A327D; L234T, L235Y, G236W, S239I, H268D, S298A and A327D; second polypeptide; D270E, K326D and K334E; D270E, K326D, A330F and K334E; D270E, K326D, A330I and K334E; D270E, K326D, A330Y and K334E; D270E, K326D, A330H and K334E; P238D, E233D, G237D, H268D, P271G, Y296D and A330R; P238D, G237D, H268D, P271G, Y296D and A330R; P238D, G237D, H268E, P271G, Y296D and A330R; P238D, E233D, G237D, H268D, P271G, Y296D, A330R and I332T; P238D, E233D, G237D, V264I, S267G, H268E, P271G and A330R; P238D, E233D, G237D, V264I, S267A, H268E, P271G and A330R; P238D, E233D, G237D, S267A, H268E, P271G, Y296D, A330R and I332T; P238D, G237D, S267A, H268E, P271G, Y296D, A330R and I332T; P238D, E233D, G237D, V264I, S267A, H268E and P271G; P238D, E233D, G237D, V264I, S267A, H268E, P271G, Y296D and A330R; P238D, E233D, G237D, V264I, S267A, H268E, P271G, Y296D, A330R and P396M; P238D, E233D, G237D, V264I, S267A, H268E, P271G, Y296D, A330R and P396L; P238D, G237D, V264I, S267A, H268E, P271G and A330R; P238D, G237D, V264I, S267A, H268E, P271G, Y296D and A330R; P238D, V264I, S267A, H268E and P271G; P238D, V264I, S267A, H268E, P271G and Y296D; P238D, G237D, S267A, H268E, P271G, Y296D and A330R; P238D, G237D, S267G, H268E, P271G, Y296D and A330R; P238D, E233D, G237D, V264I, S267A, H268E, P271G, A330R and P396M; P238D, E233D, G237D, V264I, S267A, H268E, P271G, A330R and P396L; P238D, E233D, G237D, V264I, S267A, H268E, P271G, Y296D, A327G, A330R and P396M; P238D, E233D, G237D, V264I, S267A, H268E, P271G, E272D and Y296D; P238D, G237D, V264I, S267A, H268E, P271G, E272P and A330R; P238D, G237D, V264I, S267A, H268E, P271G, E272P, Y296D and A330R; P238D, E233D, V264I, S267A, H268E and P271G; P238D, G237D, S267E, H268D, P271G, Y296D and A330R; P238D, V264I, S267A, H268E, P271G, E272D and Y296D; P238D, E233D, V264I, S267A, H268E, P271G and Y296D; P238D, E233D, L234Y, L235F, G237D, V264I, D265E, V266F, S267A, H268D, E269D, P271G, E272D, K274Q, Y296D, K326A, A327G, A330K, P331S, I332K, E333K, K334R, R355A, D356E, L358M, P396A, K409R and Q419E; G237Q, P238D, F241M, Y296E, A330H and S324H; G237Q, P238D, F241M, H268P, Y296E and A330H; G237Q, P238D, L235F, F241M, Y296E and S324H; G237Q, P238D, L235F, F241M, H268P and Y296E; G237Q, P238D, F241M, H268P, Y296E and S324H; G237Q, P238D, L235F, F241M, H268P, Y296E and S324H; G237Q, P238D, L235F, F241M, Y296E, S324H and A330H; G237Q, P238D, L235F, F241M, H268P, Y296E and A330H; G237Q, P238D, F241M, H268P, Y296E, S324H and A330H; G237Q, P238D, E233D, V264I, S267R, H268P, P271G and Y296E; G237Q, P238D, F241M and Y296E; G237Q, P238D, F241M, Y296E and A330H; G237Q, P238D, L235F, F241M and Y296E; G237Q, P238D, L235F, F241M, Y296E and A330H; G237Q and P238D; P238D and F241M; P238D and F241L; P238D and H268P; P238D and Q295V; P238D and Y296E; P238D and Y296H; P238D and S298M; P238D and S324N; P238D and S324H; P238D and A330H; P238D and A330Y; P238D and F241M, H268P, Y296E and S324H; G237Q, P238D, F241M, Y296E and A330H; L235F, G237Q, P238D, F241M and Y296E; P238D, P271G and E233D; P238D, P271G and L234R; P238D, P271G and G237D; P238D, P271G and G237K; P238D, P271G and V264I; P238D, P271G and S267A; P238D, P271G and H268E; P238D, P271G and H268P; P238D, P271G and Y296D; P238D, P271G and Y296E; P238D, P271G, E233D, L234K, V264I, S267A and H268E; P238D, P271G, E233D, L234R, V264I, S267A and H268E; P238D, P271G, E233D, G237K, V264I, S267A and H268E; P238D, P271G, E233D, V264I, D265N, S267A and H268E; P238D, P271G, E233D, V264I, S267R and H268E; P238D, P271G, E233D, G237D, V264I, S267Y, H268E, Y296D, A330R and P396M; P238D, P271G, E233D, G237D, V264I, S267A, H268E, Y296D/Y296A, A330R and P396M; P238D, P271G, E233D, V264I, S267R, H268E and Y296E; P238D, P271G, E233D, V264I, S267R and H268P; P238D, P271G, E233D, F241M, V264I, S267R and H268E; P238D, P271G, E233D, V264I, S267R, H268P and Y296E; P238D, P271G, E233D, G237Q, V264I, S267R, H268P and Y296E; E233D, G237D, P238D, H268D, P271G, and A330R.
15. The polypeptide of any one of embodiments 1-10, wherein the Fc region selectively binds to FcγRIIb over FcγRIIa.
16. The polypeptide of embodiment 15, wherein the Fc region that selectively binds to FcγRIIb over FcγRIIa comprises a mutation, mutations, or sets of mutations, as compared to the wild-type Fc region.
17. The polypeptide of embodiment 16, wherein the mutation, mutations, or set of mutations is selected from P238D; P238D and E233D; P238D and L234W; P238D and L234Y; P238D and G237W; P238D and G237F; P238D and G237A; P238D and G237D; P238D and G237E; P238D and G237L; P238D and G237M; P238D and G237Y; P238D and S239D; P238D and S267V; P238D and S267Q; P238D and S267A; P238D and H268N; P238D and H268D; P238D and H268E; P238D and P271G; P238D and Y296D; P238D and V323I; P238D and V323L; P238D and V323M; P238D and K326L; P238D and K326Q; P238D and K326E; P238D and K326M; P238D and K326D; P238D and K326S; P238D and K326T; P238D and K326A; P238D and K326N; P238D and L328E; P238D and A330K; P238D and A330R; P238D and A330M; S239P; S239P and P230E; S239P and A231D; S239P and P232E; S239P and P238E; S239P, P230E and A231D; S239P, P230E and P232E; S239P, P230E and P238E; S239P, P230E, A231D and P232E; S239P, P230E, A231D and P238E; S239P, P230E, A231D, P232E and P238E; S239P, A231D and P232E; S239P, A231D and P238E; S239P, A231D, P232E and P238E; S239P, P232E and P238E; S267E; S267D; S267E and L328F; G236D and S267E; S239D and S267E; S239D and I332E; K409E; L368K; S364D and K370G; S364Y and K370R; S364D; Y349K; K409D; K392E; D399K; S364E; L368E and K409E; S364E and F405A; Y349K and T394F; S364H and Y349K; P395T, V397S and F405A; T394F; T394S, P395V, P396T, V397E and F405S; V397S and F405A; S364H, D401K and F405A; Y349T, T394F and T411E; L351K, S364H and D401K; Y349T, L351E and T411E; S364H; Y349T; S364H and D401K; Y349T and T411E; S364H and T394F; Y349T and F405A; S364H and F405A; Y349T and T394F; F405A; S364E and T394F; Y349K and F405A; V397T and F405S; S364E and F405S; Y349K and T394Y; S364E, T411E and F405A; Y349K, T394F and D401K; S364E and T411E; Y349K and D401K; L351E and S364D; Y349K and L351K; L351E and S364E; Y349C and S364E; Y349K and S354C; S364H, F405A and T411E; Y349T, T394F and D401K; S364D and T394F; L235Y; L235R; G236D; L328F; L235Y, G236D, S267D and L328F; L235Y, G236D and S267D; L235Y, G236D and S267E; L235Y and G236D; L235Y, S267D and L328F; L235Y, S267E and L328F; L235Y and L328F; L235R, G236D, S267D and L328F; L235R, G236D and S267D; L235R, G236D and S267E; L235R and G236D; L235R, S267D and L328F; L235R, S267E and L328F; L235R and L328F; G236D, S267E and L328F; G236D, S267D and L328F; G236D and L328F; S267D and L328F; G236N and S267E; G236N; L234Y, L235Y, G236W, H268D and S298A; L234Y, L235Y, G236W, H268D, D270E and S298A; L234Y, L235Q, G236W, S239M, H268D, D270E and S298A; L234Y, L235Y, G236W, H268D, S298A and A327D; L234Y, L235Y, G236W, S239M, H268D, S298A and A327D; L234Y, L235Y, G236W, S239M, H268D, S298A, A327D, L328W and K334L; second IgG1 CH2 Domain; K326D, A330M and K334E; D270E, K326D, A330M and K334E; D270E, K326D, A330K and K334E; L234E, L235Y, G236W, S239M, H268D, S298A and A327D; L234S, L235Y, G236W, S239M, H268D, S298A and A327D; L235Q, G236W, S239M, H268D, D270E and S298A; L235Y, G236W, S239M, H268D, S298A and A327D; L234S, L235Q, G236W, S239M, H268D, D270E and S298A; L234F, L235Q, G236W, S239M, H268D, D270E and S298A; L234E, L235Q, G236W, S239M, H268D, D270E and S298A; L234F, L235Y, G236W, S239M, H268D, S298A and A327D; L234V, L235Q, G236W, S239M, H268D, D270E and S298A; L234D, L235Q, G236W, S239M, H268D, D270E and S298A; L234Q, L235Q, G236W, S239M, H268D, D270E and S298A; L234I, L235Q, G236W, S239M, H268D, D270E and S298A; L234M, L235Q, G236W, S239M, H268D, D270E and S298A; L234T, L235Q, G236W, S239M, H268D, D270E and S298A; L234A, L235Q, G236W, S239M, H268D, D270E and S298A; L234G, L235Q, G236W, S239M, H268D, D270E and S298A; L234H, L235Q, G236W, S239M, H268D, D270E and S298A; L234V, L235Y, G236W, S239M, H268D, S298A and A327D; L234D, L235Y, G236W, S239M, H268D, S298A and A327D; L234Q, L235Y, G236W, S239M, H268D, S298A and A327D; L234I, L235Y, G236W, S239M, H268D, S298A and A327D; L234M, L235Y, G236W, S239M, H268D, S298A and A327D; L234T, L235Y, G236W, S239M, H268D, S298A and A327D; L234A, L235Y, G236W, S239M, H268D, S298A and A327D; L234G, L235Y, G236W, S239M, H268D, S298A and A327D; L234H, L235Y, G236W, S239M, H268D, S298A and A327D; L234F, L235Q, G236W, S239I, H268D, D270E and S298A; L234E, L235Q, G236W, S239I, H268D, D270E and S298A; L234D, L235Q, G236W, S239I, H268D, D270E and S298A; L234V, L235Y, G236W, S239I, H268D, S298A and A327D; L234I and L235Y, G236W, S239I, H268D, S298A, A327D; L235Y, G236W, S239I, H268D, S298A, A327D; L234E, L235Y, G236W, S239I, H268D, S298A and A327D; L234D, L235Y, G236W, S239I, H268D, S298A and A327D; L234F, L235Y, G236W, S239I, H268D, S298A and A327D; L234T, L235Y, G236W, S239I, H268D, S298A and A327D; second polypeptide; D270E, K326D and K334E; D270E, K326D, A330F and K334E; D270E, K326D, A330I and K334E; D270E, K326D, A330Y and K334E; D270E, K326D, A330H and K334E; P238D, E233D, G237D, H268D, P271G, Y296D and A330R; P238D, G237D, H268D, P271G, Y296D and A330R; P238D, G237D, H268E, P271G, Y296D and A330R; P238D, E233D, G237D, H268D, P271G, Y296D, A330R and I332T; P238D, E233D, G237D, V264I, S267G, H268E, P271G and A330R; P238D, E233D, G237D, V264I, S267A, H268E, P271G and A330R; P238D, E233D, G237D, S267A, H268E, P271G, Y296D, A330R and I332T; P238D, G237D, S267A, H268E, P271G, Y296D, A330R and I332T; P238D, E233D, G237D, V264I, S267A, H268E and P271G; P238D, E233D, G237D, V264I, S267A, H268E, P271G, Y296D and A330R; P238D, E233D, G237D, V264I, S267A, H268E, P271G, Y296D, A330R and P396M; P238D, E233D, G237D, V264I, S267A, H268E, P271G, Y296D, A330R and P396L; P238D, G237D, V264I, S267A, H268E, P271G and A330R; P238D, G237D, V264I, S267A, H268E, P271G, Y296D and A330R; P238D, V264I, S267A, H268E and P271G; P238D, V264I, S267A, H268E, P271G and Y296D; P238D, G237D, S267A, H268E, P271G, Y296D and A330R; P238D, G237D, S267G, H268E, P271G, Y296D and A330R; P238D, E233D, G237D, V264I, S267A, H268E, P271G, A330R and P396M; P238D, E233D, G237D, V264I, S267A, H268E, P271G, A330R and P396L; P238D, E233D, G237D, V264I, S267A, H268E, P271G, Y296D, A327G, A330R and P396M; P238D, E233D, G237D, V264I, S267A, H268E, P271G, E272D and Y296D; P238D, G237D, V264I, S267A, H268E, P271G, E272P and A330R; P238D, G237D, V264I, S267A, H268E, P271G, E272P, Y296D and A330R; P238D, E233D, V264I, S267A, H268E and P271G; P238D, G237D, S267E, H268D, P271G, Y296D and A330R; P238D, V264I, S267A, H268E, P271G, E272D and Y296D; P238D, E233D, V264I, S267A, H268E, P271G and Y296D; P238D, E233D, L234Y, L235F, G237D, V264I, D265E, V266F, S267A, H268D, E269D, P271G, E272D, K274Q, Y296D, K326A, A327G, A330K, P331S, I332K, E333K, K334R, R355A, D356E, L358M, P396A, K409R and Q419E; G237Q, P238D, F241M, Y296E, A330H and S324H; G237Q, P238D, F241M, H268P, Y296E and A330H; G237Q, P238D, L235F, F241M, Y296E and S324H; G237Q, P238D, L235F, F241M, H268P and Y296E; G237Q, P238D, F241M, H268P, Y296E and S324H; G237Q, P238D, L235F, F241M, H268P, Y296E and S324H; G237Q, P238D, L235F, F241M, Y296E, S324H and A330H; G237Q, P238D, L235F, F241M, H268P, Y296E and A330H; G237Q, P238D, F241M, H268P, Y296E, S324H and A330H; G237Q, P238D, E233D, V264I, S267R, H268P, P271G and Y296E; G237Q, P238D, F241M and Y296E; G237Q, P238D, F241M, Y296E and A330H; G237Q, P238D, L235F, F241M and Y296E; G237Q, P238D, L235F, F241M, Y296E and A330H; G237Q and P238D; P238D and F241M; P238D and F241L; P238D and H268P; P238D and Q295V; P238D and Y296E; P238D and Y296H; P238D and S298M; P238D and S324N; P238D and S324H; P238D and A330H; P238D and A330Y; P238D and F241M, H268P, Y296E and S324H; G237Q, P238D, F241M, Y296E and A330H; L235F, G237Q, P238D, F241M and Y296E; P238D, P271G and E233D; P238D, P271G and L234R; P238D, P271G and G237D; P238D, P271G and G237K; P238D, P271G and V264I; P238D, P271G and S267A; P238D, P271G and H268E; P238D, P271G and H268P; P238D, P271G and Y296D; P238D, P271G and Y296E; P238D, P271G, E233D, L234K, V264I, S267A and H268E; P238D, P271G, E233D, L234R, V264I, S267A and H268E; P238D, P271G, E233D, G237K, V264I, S267A and H268E; P238D, P271G, E233D, V264I, D265N, S267A and H268E; P238D, P271G, E233D, V264I, S267R and H268E; P238D, P271G, E233D, G237D, V264I, S267Y, H268E, Y296D, A330R and P396M; P238D, P271G, E233D, G237D, V264I, S267A, H268E, Y296D/Y296A, A330R and P396M; P238D, P271G, E233D, V264I, S267R, H268E and Y296E; P238D, P271G, E233D, V264I, S267R and H268P; P238D, P271G, E233D, F241M, V264I, S267R and H268E; P238D, P271G, E233D, V264I, S267R, H268P and Y296E; P238D, P271G, E233D, G237Q, V264I, S267R, H268P and Y296E; E233D, G237D, P238D, H268D, P271G, and A330R.
18. The polypeptide of any one of embodiments 1-13, wherein the FcγRII binding effector domain binds to FcγRIIb or FcγRIIa.
19. The polypeptide of embodiment 18, wherein the FcγRII binding effector domains is an antibody.
20. The polypeptide of embodiment 19, wherein the antibody is an scFv, Fab, Fab′, and F(ab′)2.
21. The polypeptide of embodiment 1, wherein:
   the polypeptide comprises the inhibitory receptor effector domain, the Fc domain and the FcγRII binding effector domain, wherein the inhibitory receptor effector domain is an antagonist of the inhibitory receptor to which it binds, the Fc domain selectively binds to FcγRIIb, and the FcγRII binding effector domain selectively binds to FcγRIIb;
   the polypeptide comprises the inhibitory receptor effector domain and the Fc domain, wherein the inhibitory receptor effector domain is an antagonist of the inhibitory receptor to which it binds and the Fc domain selectively binds to FcγRIIb; or
   the polypeptide comprises the inhibitory receptor effector domain and the FcγRII binding effector domain, wherein the inhibitory receptor effector domain is an antagonist of the inhibitory receptor to which it binds and the FcγRII binding effector domain selectively binds to FcγRIIb.
22. The polypeptide of embodiment 21, wherein the polypeptide comprises the inhibitory receptor effector domain, the Fc domain and the FcγRII binding effector domain, wherein the inhibitory receptor effector domain is an antagonist of the inhibitory receptor to which it binds, the Fc domain selectively binds to FcγRIIb, and the FcγRII binding effector domain selectively binds to FcγRIIb.
23. The polypeptide of any of the preceding embodiments that comprises a formula of:

R1-Linker Region A-R2

R3-Linker Region B-R4,
wherein,
   R1, R2, R3, and R4, each independently comprises an effector binding/modulating moiety, such as those provided herein; or is absent; and
   Linker Region A and Linker B, each independently comprises an Fc moiety provided that the effector binding/modulating moieties are present, and wherein the Fc moiety selectively binds to FcγRIIb.
24. The polypeptide of embodiment 23, wherein:
   R1 comprises an anti-PD-1 antibody;
   R2 comprises an anti-LAG3 antibody;
   R3 comprises an anti-PD-1 antibody; and
   R4 comprises an anti-LAG3 antibody.
25. The polypeptide of embodiment 23, wherein;
   R1 comprises an anti-LAG3 antibody;
   R2 comprises an anti-PD-1 antibody;
   R3 comprises an anti-LAG3 antibody; and
   R4 comprises an anti-PD-1 antibody.
26. The polypeptide of embodiment 23, wherein;
   R1 comprises an anti-LAG3 antibody;
   R2 comprises a FcγRIIb-binding moiety;
   R3 comprises an anti-LAG3 antibody; and
   R4 comprises a FcγRIIb-binding moiety.
27. The polypeptide of embodiment 23, wherein;
   R1 comprises a FcγRIIb-binding moiety;
   R2 comprises an anti-LAG3 antibody;
   R3 comprises a FcγRIIb-binding moiety; and
   R4 comprises an anti-LAG3 antibody.
28. The polypeptide of embodiment 23, wherein;
   R1 comprises an anti-PD-1 antibody;
   R2 comprises a FcγRIIb-binding moiety;
   R3 comprises an anti-PD-1 antibody; and
   R4 comprises a FcγRIIb-binding moiety.
29. The polypeptide of embodiment 23, wherein;
   R1 comprises a FcγRIIb-binding moiety;
   R2 comprises an anti-PD-1 antibody;
   R3 comprises a FcγRIIb-binding moiety; and
   R4 comprises an anti-PD-1 antibody.
30. The polypeptide of embodiment 23, wherein;
   R1 comprises an anti-PD-1 antibody;
   R2 is absent;
   R3 comprises an anti-LAG3 antibody; and
   R4 is absent.
31. The polypeptide of embodiment 23, wherein;
   R1 is absent;
   R2 comprises an anti-PD-1 antibody;
   R3 is absent; and
   R4 comprises an anti-LAG3 antibody.
32. The polypeptide of embodiment 23, wherein;
   R1 comprises an anti-LAG3 antibody;
   R2 is absent;
   R3 comprises an anti-PD-1 antibody; and
   R4 is absent.
33. The polypeptide of embodiment 23, wherein;
   R1 is absent;
   R2 comprises an anti-LAG3 antibody;
   R3 is absent; and
   R4 comprises an anti-PD-1 antibody.
34. The polypeptide of embodiment 23, wherein;
   R1 comprises an anti-PD-1 antibody;
   R2 comprises a FcγRIIb-binding moiety;
   R3 comprises an anti-LAG3 antibody; and
   R4 comprises a FcγRIIb-binding moiety.
35. The polypeptide of embodiment 23, wherein;
   R1 comprises a FcγRIIb-binding moiety;
   R2 comprises an anti-PD-1 antibody; and
   R3 comprises a FcγRIIb-binding moiety;
   R4 comprises an anti-LAG3 antibody.
36. The polypeptide of embodiment 23, wherein;
   R1 comprises an anti-LAG3 antibody;
   R2 comprises a FcγRIIb-binding moiety;
   R3 comprises an anti-PD-1 antibody; and
   R4 comprises a FcγRIIb-binding moiety.
37. The polypeptide of embodiment 23, wherein;
   R1 comprises a FcγRIIb-binding moiety;
   R2 comprises an anti-LAG3 antibody;
   R3 comprises a FcγRIIb-binding moiety; and
   R4 comprises an anti-PD-1 antibody.
38. The polypeptide of any embodiment 23, wherein Linker Region A and Linker B are both present.
39. The polypeptide of any embodiment 23, wherein Linker Region A and Linker B are both absent.
40. The polypeptide of embodiment 1, wherein the polypeptide comprises an inhibitory effector domain, a Fc region and a FcγRII binding effector domain, wherein:
   the inhibitory effector domain is anti-PD-1 agonist antibody;
   the Fc region is a Fc polypeptide that selectively binds to FcγRIIb; and
   the FcγRII binding effector domain is an antibody that selectively binds to FcγRIIb.
41. The polypeptide of embodiment 1, wherein the polypeptide comprises an inhibitory effector domain, a Fc region and a FcγRII binding effector domain, wherein:
   the inhibitory effector domain is anti-LAG-3 agonist antibody;
   the Fc region is a Fc polypeptide that selectively binds to FcγRIIb; and
   the FcγRII binding effector domain is an antibody that selectively binds to FcγRIIb.
42. The polypeptide of embodiment 1, wherein the polypeptide comprises an inhibitory effector domain, a Fc region and a FcγRII binding effector domain, wherein:
   the inhibitory effector domain comprises a first inhibitory effector domain and a second inhibitory effector domain, wherein the first inhibitory effector domain is anti-PD-1 agonist antibody and the second inhibitory effector domain is anti-LAG-3 antibody;
   the Fc region is a Fc polypeptide that selectively binds to FcγRIIb; and
   the FcγRII binding effector domain is an antibody that selectively binds to FcγRIIb.
43. The polypeptide of embodiment 1, wherein the polypeptide comprises an inhibitory effector domain and a Fc region that selectively binds to FcγRIIb, wherein the inhibitory effector domain is anti-PD-1 agonist antibody.
44. The polypeptide of embodiment 1, wherein the polypeptide comprises an inhibitory effector domain and a Fc region that selectively binds to FcγRIIb, wherein the inhibitory effector domain is anti-LAG-3 agonist antibody.
45. The polypeptide of embodiment 1, wherein the polypeptide comprises an inhibitory effector domain and a Fc region that selectively binds to FcγRIIb, wherein the inhibitory effector domain comprises a first inhibitory effector domain and a second inhibitory effector domain, wherein the first inhibitory effector domain is anti-PD-1 agonist antibody and the second inhibitory effector domain is anti-LAG-3 antibody.
46. The polypeptide of embodiment 1, wherein the polypeptide comprises an inhibitory effector domain and a FcγRII binding effector domain, wherein the inhibitory effector domain is anti-PD-1 agonist antibody and the FcγRII binding effector domain is an antibody that selectively binds to FcγRIIb.
47. The polypeptide of embodiment 1, wherein the polypeptide comprises an inhibitory effector domain and a FcγRII binding effector domain, wherein the inhibitory effector domain is anti-LAG-3 agonist antibody and the FcγRII binding effector domain is an antibody that selectively binds to FcγRIIb.
48. The polypeptide of embodiment 1, wherein the polypeptide comprises an inhibitory effector domain and a FcγRII binding effector domain, wherein:
   the inhibitory effector domain comprises a first inhibitory effector domain and a second inhibitory effector domain, wherein the first inhibitory effector domain is anti-PD-1 agonist antibody and the second inhibitory effector domain is anti-LAG-3 antibody;
   and the FcγRII binding effector domain is an antibody that selectively binds to FcγRIIb.
49. A pharmaceutical composition comprising a polypeptide of any one of the preceding embodiments and a pharmaceutically acceptable excipient.
50. A method of treating an autoimmune disorder in a subject, the method comprising administering a polypeptide of any one of embodiments 1-48, or a pharmaceutical composition of embodiment 49.
51. The method of embodiment 50, wherein the autoimmune disorder is myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (CKD), autoimmune hepatitis, primary biliary cirrhosis, primary sclerosing cholangitis, antisynthetase syndrome, alopecia areata, autoimmune angioedema, autoimmune progesterone dermatitis, overlap connective tissues disease syndromes, polymyalgia rheumatic, autoimmune urticaria, bullous pemphigoid, cicatricial pemphigoid, dermatitis herpetiformis, discoid lupus erythematosus, epidermolysis bullosa acquisita, erythema nodosum, anti-neutrophil cytoplasmic antibody associated vasculitis, Henoch-Schonlein purpura, Cogan's syndrome, Buerger's disease, Susan's disease, immune complex vasculitis, primary angiitis of the CNS, gestational pemphigoid, hidradenitis suppurativa, lichen planus, lichen sclerosus, linear iga disease (lad), morphea, pemphigus vulgaris, pityriasis lichenoides et varioliformis acuta, mucha-habermann disease, psoriasis, systemic scleroderma, vitiligo, Addison's disease, autoimmune polyendocrine syndrome (APS) type 1, autoimmune polyendocrine syndrome (APS) type 2, juvenile idiopathic arthritis, juvenile dermatomyositis, autoimmune brain disease, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjögren's syndrome, autoimmune enteropathy, Coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, lyme disease (chronic), mixed connective tissue disease (MCTD), palindromic rheumatism, Parry Romberg syndrome, Parsonage-Turner syndrome, psoriatic arthritis, IBD-associated arthritis, reactive arthritis, relapsing polychondritis, retroperitoneal fibrosis, rheumatic fever, rheumatoid arthritis, autoimmune complications of immune checkpoint inhibitors (IRAEs), sarcoidosis, neurosarcoidosis, Schnitzler syndrome, systemic lupus erythematosus (SLE), undifferentiated connective tissue disease (UCTD), dermatomyositis, IgG4 related disease, fibromyalgia, antiphospholipid syndrome, inclusion body myositis, myositis, myasthenia gravis, neuromyotonia, paraneoplastic cerebellar degeneration, polymyositis, acute disseminated encephalomyelitis (ADEM), adult onset Still's disease, acute motor axonal neuropathy, anti-N-Methyl-D-Aspartate (anti-NMDA) receptor encephalitis, warm antibody hemolytic anemia (wAIHA), immune thrombocytopenia, immune thrombotic thrombocytopenia, thrombotic thrombocytopenia, pernicious anemia, aplastic anemia, Evan's syndrome, autoimmune neutropenia, acquired von Willibrand syndrome, recurring fetal loss, Rh mismatch, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, primary biliary sclerosis, glomerulonephritis, glomerular basement membrane disease, multiple sclerosis, Oshtoran syndrome, pediatric autoimmune neuropsychiatric disorder associated with Streptococcus (PANDAS), progressive inflammatory neuropathy, cutaneous lupus erythematosus, restless leg syndrome, pemphigus foliaceus including fogo selvage, transplantation, antibody-mediated rejection, alloantibody hypersensitization, xenoantibody mediated rejection, solid organ rejection, graft vs host disease acute and chronic, stiff person syndrome, Sydenham chorea, transverse myelitis, autoimmune retinopathy, autoimmune uveitis, uveitis, Cogan syndrome, Graves ophthalmopathy, amyotrophic lateral sclerosis (ALS), Parkinson's disease, autoimmune encephalitis, CNS vasculitis, chronic idiopathic demyelinating polyneuropathy (CIDP), keratitis, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, rheumatic heart disease, chronic rhinosinusitis with nasal polyps, allergic bronchoplmonary mycosis, hypersensitivity pneumonitis, rheumatoid arthritis-associated interstitial lung disease (RA-ILD), nonspecific interstitial pneumonia, allergic asthma, infectious disease/vaccination, antibody dependent enhancement (as with dengue virus infection), chronic meningitis, anti-myelin oligodendrocyte glycoprotein (MOG) disease, activated-DLBCL, anti-drug antibody, anti-gene therapy vector antibody (anti-AAV antibody), antibody to therapeutic biologic agents (cytokines, monoclonal antibodies, enzymes, coagulation factors), autoimmune inner ear disease (AIED), Meniere's disease, Behcet's disease, eosinophilic granulomatosis with polyangiitis (EGPA), giant cell arteritis, polyglandular autoimmune endocrine syndromes, granulmatosis with polyangiitis (GPA), IgA vasculitis (IgAV), Kawasaki's disease, leukocytoclastic vasculitis, lupus vasculitis, rheumatoid vasculitis, microscopic polyangiitis (VIPA), polyarteritis nodosa (PAN), polymyalgia rheumaticia, vasculitis, primary immune deficiency, pyoderma gangrenosum, agammaglobulinemia, anyloidosis, anyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Dego' deiase, eczema, eosinophilic gastroenteritis, eosinophilic pneumonia, erythroblastosis fetalsis, fibrodysplasia ossificans progressive, gastrointestinal pemphigoid, hypogammaglobulinemia, idiopathic giant-cell myocarditis, idiopathic pulmonary fibrosis, IgA nephropathy, immunoregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, or any combination thereof.
52. A method of treating cancer in a subject, the method comprising administering a polypeptide of any one of embodiments 1-48, or a pharmaceutical composition comprising of embodiment 49.
53. The method of embodiment 52, wherein the cancer is a solid of liquid tumor, such as but not limited to, lung cancer, breast, cancer, brain cancer, esophageal cancer, pancreatic cancer, hematopoietic cancer, lymphoid cancer, skin cancer, head and neck cancer, genitourinary cancer, blood cancer, and any combination thereof
54. A method of modulating two types of cells with a polypeptide, the method comprising contacting the two types of cells, with a polypeptide of any one of embodiments 1-48.
55. The method of embodiment 54, wherein one cell is a T-cell, NK Cell, Dendritic cell, and the like and the second cell is a B-Cell, an antigen presenting cell (APC), or a myeloid cell.
56. A method of modulating the activity of two types of cells in a subject, the method comprising administering to the subject a polypeptide of any one of embodiments 1-48, or a pharmaceutical composition of embodiment 49.
57. The method of embodiment 56, wherein one cell is a T-cell, NK Cell, Dendritic cell, and the like and the second cell is a B-Cell, an antigen presenting cell (APC), or a myeloid cell.
58. A method of inhibiting i) an activated immune cell (e.g. T-cell); and ii) the activity of a B-Cell, an antigen presenting cell (APC), or a myeloid cell, the method comprising administering to a subject or contacting the activated immune cell and the B Cell or antigen presenting cell with a polypeptide of any one of embodiments 1-48, or a pharmaceutical composition of embodiment 49.
59. A method of activating or enhancing an activated immune cell (e.g. T-cell) and the activity of B-Cell, an antigen presenting cell (APC), or a myeloid cell, the method comprising administering to a subject or contacting the activated immune cell and the B Cell or antigen presenting cell with a polypeptide of any one of embodiments 1-48, or a pharmaceutical composition of embodiment 49.
60. A nucleic acid encoding the polypeptide of any of embodiments 1-48.
61. A vector comprising the nucleic acid of embodiment 60.
62. A cell comprising the nucleic acid of embodiment 60 or the vector of embodiment 61.
63. A method of making the polypeptide of any one of embodiments 1-48, the method comprising culturing a cell of embodiment 62 to make the polypeptide.

The following examples are illustrative, but not limiting, of the compounds, compositions and methods described herein. Other suitable modifications and adaptations known to those skilled in the art are within the scope of the following embodiments.

Example 1: PD-1/LAG-3 and FcγRIIb dual targeted polypeptide (i.e., targeting cells that express PD-1 and LAG-3 as well as an antibody that binds selectively to FcγRIIb) Fc is used to treat autoimmune disorder. A polypeptide comprising an antibody that is a PD-1 agonist, an antibody that is a LAG-3 agonist, and a scFv that is selective for FcγRIIb, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 2: PD-1-FcγRIIb dual targeted polypeptide is used to treat autoimmune disorder. A polypeptide comprising an antibody that is a PD-1 agonist, a scFv that is selective for FcγRIIb, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein. Example 3: PD-1-FcγRIIa dual targeted polypeptide is used to treat lung cancer. A polypeptide comprising an antibody that is a PD-1 antagonist, a scFv that is selective for FcγRIIa, which are linked with a Fc region with mutations that are selective for FcγRIIa is administered to a subject with lung cancer and the subject is treated for the autoimmune disorder. Although this example describes a Fc region with mutations that are selective for FcγRIIb, the Fc region may also not be selective, but could have the LALAGA mutations (AAA) as provided for herein.

Example 4: PD-1/LAG-3 and FcγRIIa dual targeted polypeptide (i.e., targeting cells that express PD-1 and LAG-3 as well as an antibody that binds selectively to FcγRIIa) Fc is used to treat autoimmune disorder. A polypeptide comprising an antibody that is a PD-1 antagonist, an antibody that is a LAG-3 antagonist, and a scFv that is selective for FcγRIIa, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 5: PD-1 or LAG-3, and FcγRIIb dual targeted polypeptide (i.e., targeting cells that express PD-1 or LAG-3 as well as an antibody that binds selectively to FcγRIIb) Fc is used to treat autoimmune disorder. A polypeptide comprising an antibody that is a PD-1 agonist, or an antibody that is a LAG-3 agonist, and a scFv that is selective for FcγRIIb, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 6: PD-1 or LAG-3, and FcγRIIa dual targeted polypeptide (i.e., targeting cells that express PD-1 or LAG-3 as well as an antibody that binds selectively to FcγRIIa) Fc is used to treat autoimmune disorder. A polypeptide comprising an antibody that is a PD-1 antagonist, or an antibody that is a LAG-3 antagonist, and a scFv that is selective for FcγRIIa, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 7: LAG-3-FcγRIIb dual targeted polypeptide is used to treat autoimmune disorder. A polypeptide comprising an antibody that is a LAG-3 agonist, a scFv that is selective for FcγRIIb, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 8: LAG-3-FcγRIIa dual targeted polypeptide is used to treat lung cancer. A polypeptide comprising an antibody that is a LAG-3 antagonist, a scFv that is selective for FcγRIIa, which are linked with a Fc region with mutations that are selective for FcγRIIa is administered to a subject with lung cancer and the subject is treated for the autoimmune disorder. Although this example describes a Fc region with mutations that are selective for FcγRIIb, the Fc region may also not be selective, but could have the LALAGA mutations (AAA) as provided for herein.

Example 9: PD1/LAG-3-FcγRIIb dual targeted polypeptide (i.e., targeting cells that express PD-1 and LAG-3 as well as an antibody that binds selectively to FcγRIIb) is used to treat autoimmune disorder. A polypeptide comprising an antibody that is a PD-1 agonist, an antibody that is a LAG-3 agonist, and an scFv that is selective for FcγRIIb, which are linked with a Fc region with mutations that are selective for FcγRIIb is administered to a subject with an autoimmune disorder and the subject is treated for the autoimmune disorder. Although this example describes a Fc region with mutations that are selective for FcγRIIb, the Fc region may also not be selective, but could have the LALAGA mutations (AAA) as provided for herein.

Example 10: PD1/LAG-3-FcγRIIa dual targeted polypeptide (i.e., targeting cells that express PD-1 and LAG-3 as well as an antibody that binds selectively to FcγRIIb) is used to treat lung cancer. A polypeptide comprising an antibody that is a PD-1 antagonist, an antibody that is a LAG-3 antagonist, and an scFv that is selective for FcγRIIa, which are linked with a Fc region with mutations that are selective for FcγRIIa is administered to a subject with lung cancer and the subject is treated for the autoimmune disorder. Although this example describes a Fc region with mutations that are selective for FcγRIIa, the Fc region may also not be selective, but could have the LALAGA mutations (AAA) as provided for herein.

Example 11: Benchmark anti-PD-1 and anti-LAG3 agonist antibody variable domains were used for prototype generation, and anti-RSV variable domains were used as negative control. The variable domains were fused to a panel of benchmark Fc domains with different levels of selectivity: Xencor “SELF” mutant, Chugai “V12” and P238D mutants in addition to IgG1 wild-type Fc and “AAA” low/no FcR binding Fc. Benchmark moieties included IgG1-Fc (WT), IgG1-Fc V12 (V12) which exhibits increased selectivity and affinity for FcγRIIb, IgG1-Fc P238D (P238D) which is selected for FcγRIIb, IgG1-Fc S276E L328F (SELF) which exhibits increase affinity but not selectivity for FcγRIIb, and IgG1-Fc AAA (AAA) which is Fc binding silent.

Example 12: Purified prototype antibody test articles were generated for binding and functional assays. Said test articles were expressed in Expi293F cells. Each antibody test article was purified by passing it through a 5 mL PrismA column. Target antibodies were eluted with 0.1M Glycine at pH 2.8, and neutralized immediately using 5% 1M Tris HCl at pH 8.0. The eluted samples were loaded to analytical SEC to check for monodispersity of Protein of Interest (PoI). All test articles were purified with the majority population present as monomers, as shown below.

| Test Articles | monomeric POI (%) | Yield (mg/L) |
|---|---|---|
| anti PD1 IgG1 WT | 95 | ~98 |
| anti PD1 IgG1 P238D | 96 | ~97 |
| anti PD1 IgG1 V12 | 96 | ~96 |
| anti-RSV WT | 96 | ~145 |
| anti-RSV V12 | 99 | ~106 |
| Anti-LAG3 SELF | 93 | ~72 |

Example 13: Binding between test antibodies and Fc gamma receptors, or PD-1 receptors, was analyzed using Carterra SPR. In brief, antibodies were captured on protein A/G chips at concentrations of 10 μg/mL, 1 μg/mL, and 0.1 μg/mL (in duplicates). Each solution analyte protein was injected at 5 μM for Fc gamma receptors, or at 0.5 μM for PD-1 receptors. PD-1 was used to confirm antibody integrity. Binding kinetics for each antibody against PD-1, human and monkey FcγRIIb and FcγRIIa, including human FcgRIIa-R167 and FcgRIIa-H167, were obtained. Test antibodies showed the following affinities: (1) anti-PD1 IgG1 V12 showed a KD (nM) of 2.0 to human PD-1, KD of 11 to cyno PD-1, KD of 1.8 to human FcγRIIa, and a KD of 0.03 to human FcγRIIb. Effectively, the anti-PD1 IgG1 V12 molecule binds to both PD-1 and FcγRII receptors.

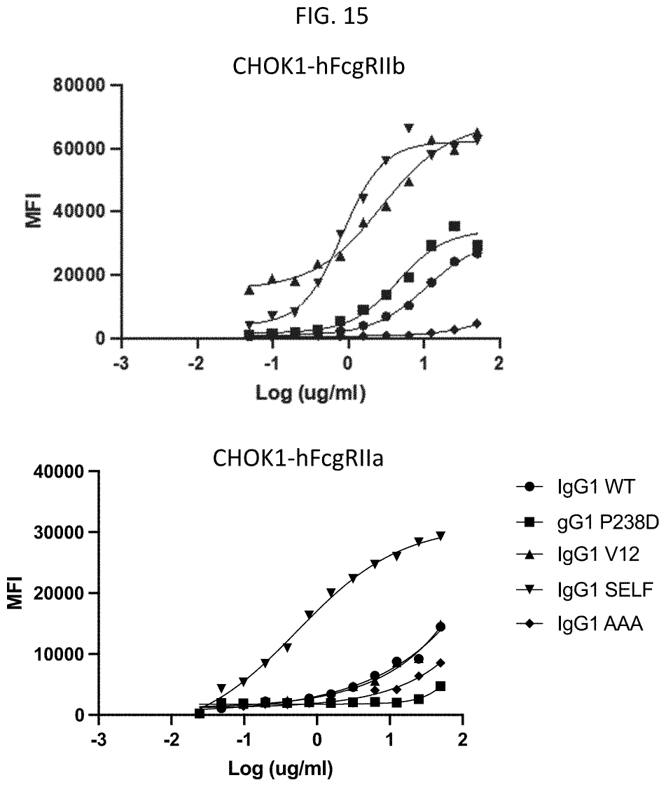

Example 14: Binding of antibodies with different affinities to Fc gamma receptors was measured using flow cytometry. In brief, CHOK1 lines overexpressing either FcγRIIb or FcγRIIa (R131) were detached, resuspended in PBS 3% FBS and incubated for 30 minutes at 4° C. with test articles (1:2 serial dilution, 11 points dilution starting from 50 ug total protein). Next, cells were washed and incubated for additional 30 minutes at 4° C. with a directly conjugated antibody recognizing the human kappa chain of the test articles. Cells were then washed, resuspended in fixation buffer for 1 hour at 4° C., washed again and resuspended in PBS prior to flow cytometry. Binding curves (log EC50) for each antibody against human FcγRIIb or FcγRIIa (R131) were obtained, and are shown in FIG. 15. EC50 values are also shown below.

| Test Articles | Log EC50 (FcgR2IIb) | Log EC50 (FcgR2IIa) |
|---|---|---|
| anti PD1 IgG1 WT | 1.026 | 9.376 |
| anti PD1 IgG1 P238D | 0.6485 | 3.438 |
| anti PD1 IgG1 V12 | 0.437 | 7.822 |
| anti-LAG3 SELF | −0.0779 | −0.2704 |
| anti-RSV AAA | 4.425 | 3.903 |

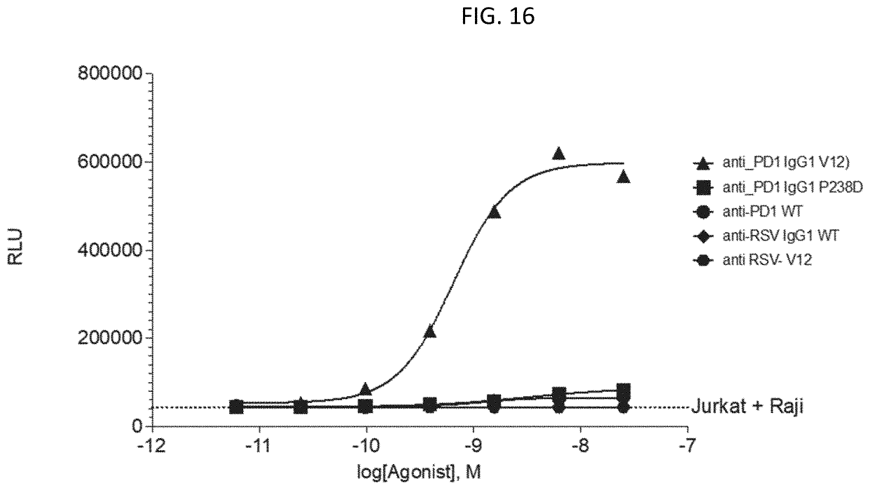

Example 15: A reporter cell line that measures fluorescence derived from SHP2 recruitment to PD1 was used as a proxy of PD-1 agonism. In brief, Raji B cells were removed from cell culture, resuspended in cell plating reagent with 3% FBS and incubated for 1 hour at 37° C. with 100 nM to 0.006 nM of test articles. Jurkat PD-1 (SHP2) reporter cells were removed from cell culture, resuspended in cell plating reagent with 3% FBS, and incubated with the Raji cells with test articles for an additional 2 hours at room temperature. Detection reagents were added to each well and luminescence was read using a plate reader. Agonism produced in reporter cell lines was enhanced by antibodies with greater affinities to FcγRIIb over the wild-type antibody control, as shown in FIG. 16 and below.

| Test Articles | Log EC50 (SHP2 recruitment) |
|---|---|
| anti PD1 IgG1 WT | −8.911 |
| anti PD1 IgG1 P238D | −8.501 |
| anti PD1 IgG1 V12 | −9.187 |
| anti-RSV-WT | Not measurable |
| anti-RSV-V12 | Not measurable |

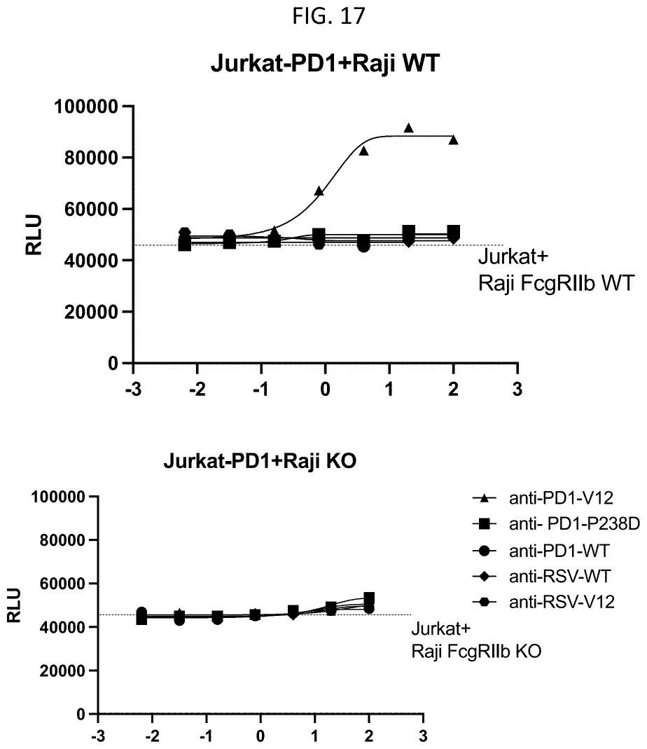

Example 16: Next, it was assessed whether the enhanced PD1 agonism observed with high affinity antibodies for FcγRIIb is dependent on FcγRIIb expression. Raji B cells expressing or deficient of FcγRIIb, and Jurkat-PD1 (SHP-2) reporter cells were removed from cell culture, resuspended in cell plating reagent with 3% FBS and incubated for 3 hour at 37° C. with 100 nM to 0.006 nM of test articles. Detection reagents were added to each well and luminescence was read using a plate reader. Agonism produced in reporter cell lines was enhanced by antibodies with greater affinities to FcγRIIb over the wild-type antibody control, as shown in FIG. 17 and below. Agonism was completely abrogated in absence of FcγRIIb. Accordingly, superior inhibitory checkpoint receptor agonism on the T cell reporter line is mediated through selective binding of Fc to FcγRIIb.

| | JURKAT-PD1 + | JURKAT-PD1 + |
|---|---|---|
| | RAJI WT | RAJI FcgRIIB |
| | (FcgRIIB+) | Knock Out |
| | Log EC50 (SHP2 | Log EC50 (SHP2 |
| Test Articles | recruitment) | recruitment) |
| anti PD1 IgG1 WT | 19.71 | Not mesuarable |
| anti PD1 IgG1 P238D | 0.1946 | Not mesuarable |
| anti PD1 IgG1 V12 | −11.06 | Not mesuarable |
| anti-RSV-WT | 0.01219 | Not mesuarable |
| anti-RSV-V12 | Not mesuarable | Not measurable |

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A polypeptide comprising:
i) an inhibitory receptor effector domain;
ii) an Fc region; and/or a FcγRII binding effector domain,
wherein the Fc region selectively binds to FcγRIIb; and
wherein the FcγRII binding effector domain selectively binds to FcγRIIb or FcγRIIa, provided that when:
i) the Fc region selectively binds to FcγRIIb, the FcγRII binding effector domain selectively binds to FcγRIIb when both the Fc region and the FcγRII binding effector domain present; or
ii) the Fc region selectively binds to FcγRIIa, the FcγRII binding effector domain selectively binds to FcγRIIa when both the Fc region and the FcγRII binding effector domain present.

2. The polypeptide of claim 1, wherein the polypeptide comprises 2 inhibitory receptor effector domains.

3-4. (canceled)

5. The polypeptide of claim 1, wherein the inhibitory receptor effector domain is an antibody.

6. (canceled)

7. The polypeptide of claim 1, wherein the inhibitor receptor effector domain binds to a receptor encoded by LAG3, PDCD1, BTLA/CD272, CD200R1, CD200R1, CD22/Siglec2, CD300A, CD300LF/CD300F, CD33/Siglec3, CD5, CD72, CEACAM 1, CLEC12A, CLEC4A, CTLA4/CD152, FCGR2B/CD32B, KIRs, KLRB1/CD161, KLRC1, KLRG1, LAIR1, LILRB1, LILRB2, LILRB4, LILRB5, NCR2/NKp44, PECAM1/CD31, PILRA, PVR/CD155, SIGLEC11, SIGLEC5, SIGLEC7, SIGLEC8, SIGLEC9, SIRPA, TIGIT, VSTM1/SIRL1, MAFA, NKG2A, CMRF35H, CD66a, CD66d, CD33, SIGLEC6, ILT2, ILT3, ILT4, ILT5, LIR8, KIR2DL, KIR2DL1, KIR3DL, SIRPa, KIR2DL2/3, KIR2DL5, KIRDL1, KIRDL2, KIRDL3, TIM3, Tactile, IRp60, NKRP1, IAP, PIR-B, CD5, 2B4, GP49B, Ly49Q, MICL, CD160, FCRL4, KIR3DL1, KIR2DL2, LILRB3, DCIR, NKRP-1D, LY49, MAIR-I, CD79a, CD79b, CD19, CD21, CD40, TLR3, CD28, CCR5, or CCR1.

8. The polypeptide of claim 1, wherein the inhibitory receptor effector domain binds to PD-1, LAG-3, or CTLA4.

9-10. (canceled)

11. The polypeptide of claim 1, wherein the Fc region selectively binds to FcγRIIb.

12. (canceled)

13. The polypeptide of claim 11, wherein the Fc region that selectively binds to FcγRIIb comprises a mutation, mutations, or a set of mutations, as compared to a wild-type Fc region.

14. The polypeptide of claim 13, wherein the mutation, mutations, or set of mutations is selected from P238D; P238D and E233D; P238D and L234W; P238D and L234Y; P238D and G237W; P238D and G237F; P238D and G237A; P238D and G237D; P238D and G237E; P238D and G237L; P238D and G237M; P238D and G237Y; P238D and S239D; P238D and S267V; P238D and S267Q; P238D and S267A; P238D and H268N; P238D and H268D; P238D and H268E; P238D and P271G; P238D and Y296D; P238D and V323I; P238D and V323L; P238D and V323M; P238D and K326L; P238D and K326Q; P238D and K326E; P238D and K326M; P238D and K326D; P238D and K326S; P238D and K326T; P238D and K326A; P238D and K326N; P238D and L328E; P238D and A330K; P238D and A330R; P238D and A330M; S239P; S239P and P230E; S239P and A231D; S239P and P232E; S239P and P238E; S239P, P230E and A231D; S239P, P230E and P232E; S239P, P230E and P238E; S239P, P230E, A231D and P232E; S239P, P230E, A231D and P238E; S239P, P230E, A231D, P232E and P238E; S239P, A231D and P232E; S239P, A231D and P238E; S239P, A231D, P232E and P238E; S239P, P232E and P238E; S267E; S267D; S267E and L328F; G236D and S267E; S239D and S267E; S239D and I332E; K409E; L368K; S364D and K370G; S364Y and K370R; S364D; Y349K; K409D; K392E; D399K; S364E; L368E and K409E; S364E and F405A; Y349K and T394F; S364H and Y349K; P395T, V397S and F405A; T394F; T394S, P395V, P396T, V397E and F405S; V397S and F405A; S364H, D401K and F405A; Y349T, T394F and T411E; L351K, S364H and D401K; Y349T, L351E and T411E; S364H; Y349T; S364H and D401K; Y349T and T411E; S364H and T394F; Y349T and F405A; S364H and F405A; Y349T and T394F; F405A; S364E and T394F; Y349K and F405A; V397T and F405S; S364E and F405S; Y349K and T394Y; S364E, T411E and F405A; Y349K, T394F and D401K; S364E and T411E; Y349K and D401K; L351E and S364D; Y349K and L351K; L351E and S364E; Y349C and S364E; Y349K and S354C; S364H, F405A and T411E; Y349T, T394F and D401K; S364D and T394F; L235Y; L235R; G236D; L328F; L235Y, G236D, S267D and L328F; L235Y, G236D and S267D; L235Y, G236D and S267E; L235Y and G236D; L235Y, S267D and L328F; L235Y, S267E and L328F; L235Y and L328F; L235R, G236D, S267D and L328F; L235R, G236D and S267D; L235R, G236D and S267E; L235R and G236D; L235R, S267D and L328F; L235R, S267E and L328F; L235R and L328F; G236D, S267E and L328F; G236D, S267D and L328F; G236D and L328F; S267D and L328F; G236N and S267E; G236N; L234Y, L235Y, G236W, H268D and S298A; L234Y, L235Y, G236W, H268D, D270E and S298A; L234Y, L235Q, G236W, S239M, H268D, D270E and S298A; L234Y, L235Y, G236W, H268D, S298A and A327D; L234Y, L235Y, G236W, S239M, H268D, S298A and A327D; L234Y, L235Y, G236W, S239M, H268D, S298A, A327D, L328W and K334L; second IgG1 CH2 Domain; K326D, A330M and K334E; D270E, K326D, A330M and K334E; D270E, K326D, A330K and K334E; L234E, L235Y, G236W, S239M, H268D, S298A and A327D; L234S, L235Y, G236W, S239M, H268D, S298A and A327D; L235Q, G236W, S239M, H268D, D270E and S298A; L235Y, G236W, S239M, H268D, S298A and A327D; L234S, L235Q, G236W, S239M, H268D, D270E and S298A; L234F, L235Q, G236W, S239M, H268D, D270E and S298A; L234E, L235Q, G236W, S239M, H268D, D270E and S298A; L234F, L235Y, G236W, S239M, H268D, S298A and A327D; L234V, L235Q, G236W, S239M, H268D, D270E and S298A; L234D, L235Q, G236W, S239M, H268D, D270E and S298A; L234Q, L235Q, G236W, S239M, H268D, D270E and S298A; L234I, L235Q, G236W, S239M, H268D, D270E and S298A; L234M, L235Q, G236W, S239M, H268D, D270E and S298A; L234T, L235Q, G236W, S239M, H268D, D270E and S298A; L234A, L235Q, G236W, S239M, H268D, D270E and S298A; L234G, L235Q, G236W, S239M, H268D, D270E and S298A; L234H, L235Q, G236W, S239M, H268D, D270E and S298A; L234V, L235Y, G236W, S239M, H268D, S298A and A327D; L234D, L235Y, G236W, S239M, H268D, S298A and A327D; L234Q, L235Y, G236W, S239M, H268D, S298A and A327D; L234I, L235Y, G236W, S239M, H268D, S298A and A327D; L234M, L235Y, G236W, S239M, H268D, S298A and A327D; L234T, L235Y, G236W, S239M, H268D, S298A and A327D; L234A, L235Y, G236W, S239M, H268D, S298A and A327D; L234G, L235Y, G236W, S239M, H268D, S298A and A327D; L234H, L235Y, G236W, S239M, H268D, S298A and A327D; L234F, L235Q, G236W, S239M, H268D, D270E and S298A; L234E, L235Q, G236W, S239M, H268D, D270E and S298A; L234D, L235Q, G236W, S239M, H268D, D270E and S298A; L234V, L235Y, G236W, S239M, H268D, S298A and A327D; L234I and L235Y, G236W, S239I, H268D, S298A, A327D; L235Y, G236W, S239I, H268D, S298A, A327D; L234E, L235Y, G236W, S239I, H268D, S298A and A327D; L234D, L235Y, G236W, S239I, H268D, S298A and A327D; L234F, L235Y, G236W, S239I, H268D, S298A and A327D; L234T, L235Y, G236W, S239I, H268D, S298A and A327D; second polypeptide; D270E, K326D and K334E; D270E, K326D, A330F and K334E; D270E, K326D, A330I and K334E; D270E, K326D, A330Y and K334E; D270E, K326D, A330H and K334E; P238D, E233D, G237D, H268D, P271G, Y296D and A330R; P238D, G237D, H268D, P271G, Y296D and A330R; P238D, G237D, H268E, P271G, Y296D and A330R; P238D, E233D, G237D, H268D, P271G, Y296D, A330R and I332T; P238D, E233D, G237D, V264I, S267G, H268E, P271G and A330R; P238D, E233D, G237D, V264I, S267A, H268E, P271G and A330R; P238D, E233D, G237D, S267A, H268E, P271G, Y296D, A330R and I332T; P238D, G237D, S267A, H268E, P271G, Y296D, A330R and I332T; P238D, E233D, G237D, V264I, S267A, H268E and P271G; P238D, E233D, G237D, V264I, S267A, H268E, P271G, Y296D and A330R; P238D, E233D, G237D, V264I, S267A, H268E, P271G, Y296D, A330R and P396M; P238D, E233D, G237D, V264I, S267A, H268E, P271G, Y296D, A330R and P396L; P238D, G237D, V264I, S267A, H268E, P271G and A330R; P238D, G237D, V264I, S267A, H268E, P271G, Y296D and A330R; P238D, V264I, S267A, H268E and P271G; P238D, V264I, S267A, H268E, P271G and Y296D; P238D, G237D, S267A, H268E, P271G, Y296D and A330R; P238D, G237D, S267G, H268E, P271G, Y296D and A330R; P238D, E233D, G237D, V264I, S267A, H268E, P271G, A330R and P396M; P238D, E233D, G237D, V264I, S267A, H268E, P271G, A330R and P396L; P238D, E233D, G237D, V264I, S267A, H268E, P271G, Y296D, A327G, A330R and P396M; P238D, E233D, G237D, V264I, S267A, H268E, P271G, E272D and Y296D; P238D, G237D, V264I, S267A, H268E, P271G, E272P and A330R; P238D, G237D, V264I, S267A, H268E, P271G, E272P, Y296D and A330R; P238D, E233D, V264I, S267A, H268E and P271G; P238D, G237D, S267E, H268D, P271G, Y296D and A330R; P238D, V264I, S267A, H268E, P271G, E272D and Y296D; P238D, E233D, V264I, S267A, H268E, P271G and Y296D; P238D, E233D, L234Y, L235F, G237D, V264I, D265E, V266F, S267A, H268D, E269D, P271G, E272D, K274Q, Y296D, K326A, A327G, A330K, P331S, 1332K, E333K, K334R, R355A, D356E, L358M, P396A, K409R and Q419E; G237Q, P238D, F241M, Y296E, A330H and S324H; G237Q, P238D, F241M, H268P, Y296E and A330H; G237Q, P238D, L235F, F241M, Y296E and S324H; G237Q, P238D, L235F, F241M, H268P and Y296E; G237Q, P238D, F241M, H268P, Y296E and S324H; G237Q, P238D, L235F, F241M, H268P, Y296E and S324H; G237Q, P238D, L235F, F241M, Y296E, S324H and A330H; G237Q, P238D, L235F, F241M, H268P, Y296E and A330H; G237Q, P238D, F241M, H268P, Y296E, S324H and A330H; G237Q, P238D, E233D, V264I, S267R, H268P, P271G and Y296E; G237Q, P238D, F241M and Y296E; G237Q, P238D, F241M, Y296E and A330H; G237Q, P238D, L235F, F241M and Y296E; G237Q, P238D, L235F, F241M, Y296E and A330H; G237Q and P238D; P238D and F241M; P238D and F241L; P238D and H268P; P238D and Q295V; P238D and Y296E; P238D and Y296H; P238D and S298M; P238D and S324N; P238D and S324H; P238D and A330H; P238D and A330Y; P238D and F241M, H268P, Y296E and S324H; G237Q, P238D, F241M, Y296E and A330H; L235F, G237Q, P238D, F241M and Y296E; P238D, P271G and E233D; P238D, P271G and L234R; P238D, P271G and G237D; P238D, P271G and G237K; P238D, P271G and V264I; P238D, P271G and S267A; P238D, P271G and H268E; P238D, P271G and H268P; P238D, P271G and Y296D; P238D, P271G and Y296E; P238D, P271G, E233D, L234K, V264I, S267A and H268E; P238D, P271G, E233D, L234R, V264I, S267A and H268E; P238D, P271G, E233D, G237K, V264I, S267A and H268E; P238D, P271G, E233D, V264I, D265N, S267A and H268E; P238D, P271G, E233D, V264I, S267R and H268E; P238D, P271G, E233D, G237D, V264I, S267Y, H268E, Y296D, A330R and P396M; P238D, P271G, E233D, G237D, V264I, S267A, H268E, Y296D/Y296A, A330R and P396M; P238D, P271G, E233D, V264I, S267R, H268E and Y296E; P238D, P271G, E233D, V264I, S267R and H268P; P238D, P271G, E233D, F241M, V264I, S267R and H268E; P238D, P271G, E233D, V264I, S267R, H268P and Y296E; P238D, P271G, E233D, G237Q, V264I, S267R, H268P and Y296E; E233D, G237D, P238D, H268D, P271G, and A330R.

15-17. (canceled)

18. The polypeptide of claim 1, wherein the FcγRII binding effector domain binds to FcγRIIb or FcγRIIa.

19. The polypeptide of claim 18, wherein the FcγRII binding effector domains is an antibody.

20. (canceled)

21. The polypeptide of claim 1, wherein:
the polypeptide comprises the inhibitory receptor effector domain, the Fc domain and the FcγRII binding effector domain, wherein the inhibitory receptor effector domain is an antagonist of the inhibitory receptor to which it binds, the Fc domain selectively binds to FcγRIIb, and the FcγRII binding effector domain selectively binds to FcγRIIb;
the polypeptide comprises the inhibitory receptor effector domain and the Fc domain, wherein the inhibitory receptor effector domain is an antagonist of the inhibitory receptor to which it binds and the Fc domain selectively binds to FcγRIIb; or
the polypeptide comprises the inhibitory receptor effector domain and the FcγRII binding effector domain, wherein the inhibitory receptor effector domain is an antagonist of the inhibitory receptor to which it binds and the FcγRII binding effector domain selectively binds to FcγRIIb.

22. (canceled)

23. The polypeptide of claim 1 that comprises a formula of:
R1-Linker Region A-R2
R3-Linker Region B-R4,
wherein,
R1, R2, R3, and R4, each independently comprises an effector binding/modulating moiety, such as those provided herein; or is absent; and
Linker Region A and Linker B, each independently comprises an Fc moiety provided that the effector binding/modulating moieties are present, and wherein the Fc moiety selectively binds to FcγRIIb.

24. The polypeptide of claim 23, wherein:
R1 comprises an anti-PD-1 antibody, R2 comprises an anti-LAG3 antibody, R3 comprises an anti-PD-1 antibody, and R4 comprises an anti-LAG3 antibody;
R1 comprises an anti-LAG3 antibody, R2 comprises an anti-PD-1 antibody, R3 comprises an anti-LAG3 antibody, and R4 comprises an anti-PD-1 antibody;
R1 comprises an anti-LAG3 antibody, R2 comprises a FcγRIIb-binding moiety, R3 comprises an anti-LAG3 antibody, and R4 comprises a FcγRIIb-binding moiety;
R1 comprises a FcγRIIb-binding moiety, R2 comprises an anti-LAG3 antibody, R3 comprises a FcγRIIb-binding moiety, and R4 comprises an anti-LAG3 antibody;
R1 comprises an anti-PD-1 antibody, R2 comprises a FcγRIIb-binding moiety, R3 comprises an anti-PD-1 antibody, and R4 comprises a FcγRIIb-binding moiety;
R1 comprises a FcγRIIb-binding moiety, R2 comprises an anti-PD-1 antibody, R3 comprises a FcγRIIb-binding moiety, and R4 comprises an anti-PD-1 antibody;
R1 comprises an anti-PD-1 antibody, R2 is absent, R3 comprises an anti-LAG3 antibody, and R4 is absent;
R1 is absent, R2 comprises an anti-PD-1 antibody, R3 is absent, and R4 comprises an anti-LAG3 antibody;
R1 comprises an anti-LAG3 antibody, R2 is absent, R3 comprises an anti-PD-1 antibody, and R4 is absent;
R1 is absent, R2 comprises an anti-LAG3 antibody, R3 is absent, and R4 comprises an anti-PD-1 antibody;
R1 comprises an anti-PD-1 antibody, R2 comprises a FcγRIIb-binding moiety, R3 comprises an anti-LAG3 antibody, and R4 comprises a FcγRIIb-binding moiety;
R1 comprises a FcγRIIb-binding moiety, R2 comprises an anti-PD-1 antibody, R3 comprises a FcγRIIb-binding moiety, and R4 comprises an anti-LAG3 antibody.
R1 comprises an anti-LAG3 antibody, R2 comprises a FcγRIIb-binding moiety, R3 comprises an anti-PD-1 antibody, and R4 comprises a FcγRIIb-binding moiety; or
R1 comprises a FcγRIIb-binding moiety, R2 comprises an anti-LAG3 antibody, R3 comprises a FcγRIIb-binding moiety, and R4 comprises an anti-PD-1 antibody.

25-48. (canceled)

49. A pharmaceutical composition comprising a polypeptide of claim 1 and a pharmaceutically acceptable excipient.

50. A method of treating an autoimmune disorder in a subject, the method comprising administering a polypeptide of claim 1.

51. The method of claim 50, wherein the autoimmune disorder is myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (CKD), autoimmune hepatitis, primary biliary cirrhosis, primary sclerosing cholangitis, antisynthetase syndrome, alopecia areata, autoimmune angioedema, autoimmune progesterone dermatitis, overlap connective tissues disease syndromes, polymyalgia rheumatic, autoimmune urticaria, bullous pemphigoid, cicatricial pemphigoid, dermatitis herpetiformis, discoid lupus erythematosus, epidermolysis bullosa acquisita, erythema nodosum, anti-neutrophil cytoplasmic antibody associated vasculitis, Henoch-Schonlein purpura, Cogan's syndrome, Buerger's disease, Susan's disease, immune complex vasculitis, primary angiitis of the CNS, gestational pemphigoid, hidradenitis suppurativa, lichen planus, lichen sclerosus, linear iga disease (lad), morphea, pemphigus vulgaris, pityriasis lichenoides et varioliformis acuta, mucha-habermann disease, psoriasis, systemic scleroderma, vitiligo, Addison's disease, autoimmune polyendocrine syndrome (APS) type 1, autoimmune polyendocrine syndrome (APS) type 2, juvenile idiopathic arthritis, juvenile dermatomyositis, autoimmune brain disease, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjögren's syndrome, autoimmune enteropathy, Coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, lyme disease (chronic), mixed connective tissue disease (MCTD), palindromic rheumatism, Parry Romberg syndrome, Parsonage-Turner syndrome, psoriatic arthritis, IBD-associated arthritis, reactive arthritis, relapsing polychondritis, retroperitoneal fibrosis, rheumatic fever, rheumatoid arthritis, autoimmune complications of immune checkpoint inhibitors (IRAEs), sarcoidosis, neurosarcoidosis, Schnitzler syndrome, systemic lupus erythematosus (SLE), undifferentiated connective tissue disease (UCTD), dermatomyositis, IgG4 related disease, fibromyalgia, antiphospholipid syndrome, inclusion body myositis, myositis, myasthenia gravis, neuromyotonia, paraneoplastic cerebellar degeneration, polymyositis, acute disseminated encephalomyelitis (ADEM), adult onset Still's disease, acute motor axonal neuropathy, anti-N-Methyl-D-Aspartate (anti-NMDA) receptor encephalitis, warm antibody hemolytic anemia (wAIHA), immune thrombocytopenia, immune thrombotic thrombocytopenia, thrombotic thrombocytopenia, pernicious anemia, aplastic anemia, Evan's syndrome, autoimmune neutropenia, acquired von Willibrand syndrome, recurring fetal loss, Rh mismatch, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, primary biliary sclerosis, glomerulonephritis, glomerular basement membrane disease, multiple sclerosis, Oshtoran syndrome, pediatric autoimmune neuropsychiatric disorder associated with Streptococcus (PANDAS), progressive inflammatory neuropathy, cutaneous lupus erythematosus, restless leg syndrome, pemphigus foliaceus including fogo selvage, transplantation, antibody-mediated rejection, alloantibody hypersensitization, xenoantibody mediated rejection, solid organ rejection, graft vs host disease acute and chronic, stiff person syndrome, Sydenham chorea, transverse myelitis, autoimmune retinopathy, autoimmune uveitis, uveitis, Cogan syndrome, Graves ophthalmopathy, amyotrophic lateral sclerosis (ALS), Parkinson's disease, autoimmune encephalitis, CNS vasculitis, chronic idiopathic demyelinating polyneuropathy (CIDP), keratitis, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, rheumatic heart disease, chronic rhinosinusitis with nasal polyps, allergic bronchoplmonary mycosis, hypersensitivity pneumonitis, rheumatoid arthritis-associated interstitial lung disease (RA-ILD), nonspecific interstitial pneumonia, allergic asthma, infectious disease/vaccination, antibody dependent enhancement (as with dengue virus infection), chronic meningitis, anti-myelin oligodendrocyte glycoprotein (MOG) disease, activated-DLBCL, anti-drug antibody, anti-gene therapy vector antibody (anti-AAV antibody), antibody to therapeutic biologic agents (cytokines, monoclonal antibodies, enzymes, coagulation factors), autoimmune inner ear disease (AIED), Meniere's disease, Behcet's disease, eosinophilic granulomatosis with polyangiitis (EGPA), giant cell arteritis, polyglandular autoimmune endocrine syndromes, granulmatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, pyoderma gangrenosum, agammaglobulinemia, anyloidosis, anyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Dego' deiase, eczema, eosinophilic gastroenteritis, eosinophilic pneumonia, erythroblastosis fetalsis, fibrodysplasia ossificans progressive, gastrointestinal pemphigoid, hypogammaglobulinemia, idiopathic giant-cell myocarditis, idiopathic pulmonary fibrosis, IgA nephropathy, immunoregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, or any combination thereof.

52. A method of treating cancer in a subject, the method comprising administering a polypeptide of claim 1.

53. (canceled)

54. A method of modulating two types of cells with a polypeptide, the method comprising contacting the two types of cells, with a polypeptide of claim 1.

55-57. (canceled)

58. A method of inhibiting i) an activated immune cell (e.g. T-cell); and ii) the activity of a B-Cell, an antigen presenting cell (APC), or a myeloid cell, the method comprising administering to a subject or contacting the activated immune cell and the B Cell or antigen presenting cell with a polypeptide of claim 1.

59. (canceled)

60. A nucleic acid encoding the polypeptide of claim 1.

61-64. (canceled)
